(19) United States
(12) Patent Application Publication
(10) Pub. No.: US 2018/0245065 A1
(43) Pub. Date: Aug. 30, 2018

(54) METHODS AND COMPOSITIONS FOR ENHANCING GENE EDITING

(71) Applicant: NOVARTIS AG, Basel (CH)

(72) Inventors: Robert IHRY, Cambridge, MA (US); Ajamete KAYKAS, Cambridge, MA (US); Kathleen WORRINGER, Somerville, MA (US)

(21) Appl. No.: 15/800,352

(22) Filed: Nov. 1, 2017

Related U.S. Application Data
(60) Provisional application No. 62/415,712, filed on Nov. 1, 2016.

(51) Int. Cl. C12N 15/10; C12N 9/22; C12N 15/86; C07K 16/24; C07K 14/47

(52) U.S. Cl. CPC C12N 15/102; C12N 9/22; C12N 15/86; C07K 16/24; C07K 14/4746; C12N 2710/10041; C12N 2310/3519; C12N 2310/16; C12N 2310/122; C12N 2310/14; C12N 2310/20

(57) ABSTRACT

The invention provides novel methods and compositions for enhancing gene editing.

### TECHNICAL FIELD

The present invention provides methods and compositions for enhancing gene editing.

### BACKGROUND

Gene editing systems, such as zinc finger nucleases, CRISPR/Cas systems, transcription activator-like effector nucleases (TALENs), and meganucleases, have emerged as powerful tools for drug discoveries as well as for therapeutic uses.

For example, the CRISPR/Cas9 system has been used in functional genomics studies and it is now possible to conduct genetic screens in a wide range of diploid human cells Hart et al. (2015) Cell 163: 1-12, Shalem et al. (2014) Science 343:84-87, Wang et al. (2014) Science 343: 80-84. Despite its high efficacy and wide adoption, there have been negative reports related to Cas9 in human cells, and concerns about the off-target toxicity of the technology. There is a need to find ways to decrease toxicity associated with gene editing systems and to increase gene editing efficiency.

### SUMMARY OF THE INVENTION

The present invention is based, at least in part, on the findings that inhibition of apoptosis, e.g., inhibition of TP53, reduces gene editing toxicity, such as Cas9-induced toxicity and/or greatly increased the efficiency of homology dependent repair (HDR) in cells, such as human pluripotent stem cells (hPSCs). Accordingly, provided herein are methods and compositions for decreasing toxicity of a gene editing system and/or increasing gene editing efficiency by inhibition of apoptosis, e.g., TP53. The present invention allows for the survival of highly modified cells in which multiple genomic sites are being modified in parallel. Cellular toxicity increases as the number of genomic modifications increase and the invention described herein reduces toxicity and promotes the survival of highly modified cells.

In one aspect, the present invention provides a gene editing system comprising an apoptosis inhibitor, e.g., a TP53 inhibitor. Such gene editing system can also include a nuclease or a gene editing vector.

In one aspect, the present invention provides a gene editing system comprising: (1) a TP53 inhibitor, and (2) a nuclease. The gene editing system of the present invention can be any gene editing system in combination with a TP53 inhibitor. In one aspect, the nuclease of the gene editing system of the present invention is a meganuclease, zinc finger nuclease (ZFNs), transcription activator-like effector-based nuclease (TALEN), CPF1, or Cas9.

In one aspect, the present invention provides a gene editing system comprising: (1) a TP53 inhibitor, and (2) a gene editing vector, e.g., a gene editing viral vector. In some embodiments, the gene editing vector is a recombinant adeno-associated virus (rAAV) based gene editing vector. In some embodiments, the gene editing vector is a recombinant AAV Clade F vector.

In one aspect, the gene editing system of the present invention is a Cas9 system that comprises: (i) a TP53 inhibitor, (2) a Cas9 molecule, and (3) a gRNA molecule, wherein the gRNA molecule is capable of targeting the Cas9 molecule to a target nucleic acid. In some embodiments, the Cas9 system further comprises a second gRNA molecule, and wherein the second gRNA molecule is capable of targeting the Cas9 molecule to the target nucleic acid. The gRNA molecule of the present invention can be an RNA molecule, or a DNA molecule encoding the gRNA molecule. The Cas9 molecule of the present invention can be a Cas9 polypeptide or a nucleic acid encoding a Cas9 polypeptide, and includes variants and orthologs, In one embodiment, the Cas9 molecule of the present invention is a naturally occuring Cas9 molecule of S. pyogenes. In another embodiment, the Cas9 molecule of the present invention comprises one or more mutations as compared to a naturally occuring Cas9.

In some embodiments, the gene editing system of the present invention is regulated. For example, the expression of the Cas9 molecule is regulated. In some embodiments, the expression of the Cas9 molecule is induced by using doxycycline, shield 1, 4HT, rapamycin, or Light. In some embodiments, the expression of said Cas9 molecule is inhibited by using ASV/CLV SMASHTAG.

In one aspect, the gene editing system of the present invention comprises a TP53 inhibitor, wherein the TP53 inhibitor is a protein, a nucleic acid, an antibody, a small molecule, or a gene editing system (e.g., dCas9-transcription repressor fusion) that targets TP53 and modifies its function.

In some embodiments, the TP53 inhibitor is a protein, a nucleic acid, an antibody, a small molecule, or a gene editing system (e.g., dCas9-transcription repressor fusion) that targets TP53 and inhibits its function.

In some embodiments, the TP 53 inhibitor is a protein, a nucleic acid, an antibody, a small molecule or a gene editing system (e.g., Cas9 fusion to active MDM2) that targets MDM2 and activates its function. For example, a TP53 inhibitor can be a chemical or genetic activator of MDM2 or a related ubiquitn ligase that promotes the degradation of TP53. Such TP53 inhibitors can be a recombinant MDM2 protein or variant thereof, or a nucleic acid encoding such MDM2 protein or variant, which can degrade TP53 protein and therefore inhibit p53 cellular function. In some embodiments, TP53 inhibitor can be a noncleavable MDM2 variant or a nucleic acid encoding a noncleavable MDM2 variant. For example, TP53 inhibitor can be a MDM2 variant that is resistant to Caspase 2 cleavage or a nucleic acid encoding such a MDM2 variant, e.g., a MDM2 variant containing an amino acid substitution or deletion of Asp 367 (see Oliver et al., Mol Cell. 2011 Jul. 8; 43(1):57-71). In some embodiments, TP53 inhibitor can be a hyperactive MDM2 variant or a nucleic acid encoding a hyperactive MDM2 variant. For example, TP53 inhibitor can be a MDM2-3AD construct containing two extra tandem copies of the acidic domain (AD) sequence (residues 221 to 280) as described in Cheng et al., Mol Cell Biol. 2014 August; 34(15): 2800-2810). Other hyperactive MDM2 include MDM2-S395A or MDM2-S294A, see Li et al., Cancer Cell. 2012 May 25; 21(5): 668-679.

In some embodiments, the TP53 inhibitor is a recombinant MDM2 protein or variant having an amino acid sequence selected from any one of SEQ ID NOs: 15-18.

In some embodiments, TP53 inhibitor can be a MDM2 variant or TP53 variant that enhances the interaction between TP53 and MDM2, or a nucleic acid encoding such MDM2 or TP53 variant. Such TP53 inhibitors include MDM2 variants having higher binding affinity for TP53 than wild type MDM2, or TP53 variants having higher binding affinity for MDM2 than wild type TP53. For example, TP53 variant with P27A mutation, or P12A, P13A, P27A triple mutations have been shown to bind to MDM2 at higher affinity than wild type TP53 (Borcherds et al., Nature Chemical Biology 10, 1000-1002, 2014).

In some embodiments, the TP53 inhibitor of the present invention is a nucleic acid, and wherein said nucleic acid is a DNA, mRNA, siRNA, a shRNA, a miRNA, an antiMiR or an aptamer. In one embodiment, the TP53 inhibitor is a nucleic acid comprises SEQ ID NO: 9.

In some embodiments, the TP53 inhibitor of the present invention is a protein, and wherein said protein is a TP53 variant that inhibits naturally occuring TP53 expression. In some embodiments, the TP53 variant of the present invention comprises SEQ ID NO:6, SEQ ID NO:7, or SEQ ID NO:8.

In some embodiments, the TP53 inhibitor is a small molecule. In some embodiments, the TP53 inhibitor is selected from nutlin-3; pifithrin-alpha hydrobromide; roscovitine; pifithrin-alpha, p-Nitro; pifithrin-mu; 9-hydroxyellipticine, hydrochloride; pifithrin-alpha, p-nitro, cyclic; cyclic pifithrin-alpha hydrobromide; SJ 1725550; (+)-nutlin-3; (ndas)-nutlin-3; or ReAcp53. In some embodiments, the TP53 inhibitor of the present invention is pifithrin-alpha or pifithrin-mu.

In some embodiments, the TP53 inhibitor is a fusion protein, e.g., a Cas9 fusion protein, e.g., a Cas9 fused to a heterologous effector domain that inhibits TP53. In some embodiments, such a TP53 inhibitor is a Cas9 fused to a dominant negative TP53, or a Cas9 fused to MDM2 or variant. For example, such fusion protein can have an amino acid sequence selected from any one of SEQ ID NOs: 19-24.

In one aspect, the gene editing system of the present invention further comprises a template nucleic acid. In some embodiments, the template nucleic acid comprises a circular nucleic acid, for example, the circular nucleic acid is a plasmid. In some embodiments, the gene editing system of the present invention comprises a template nucleic acid that is a linear nucleic acid. In some embodiments, the template nucleic acid comprises a double strand sequence. In some embodiments, the template nucleic acid comprises a single strand oligonucleotide.

In one aspect, the present invention provides cells comprising the gene editing system described herein. The cells can be from any subject, including human and non-human cells, e.g., from the metazoan clade and has a TP53 family member, see e.g., Belyi et al., Cold Spring Harb Perspect Biol 2010; 2:a001198. In some embodiments, the cells are human cells. In some embodiments, the cells are non-human cells, such as pig cells. In some embodiments, the cells are stem cells or primary human cells. In some embodiments, the cells are further engineered to express a chimeric antigen receptor (CAR).

In one aspect, the present invention provides compositions comprising the gene editing system described herein. In some embodiments, the present invention provides pharmaceutical composition comprising such composition and a pharmaceutically acceptable carrier. In some embodiments, the pharmaceutical composition further comprises instructions for use to treat a disorder. The present invention further provides kits comprising the gene editing systems described herein.

In one aspect, the present invention provides vectors comprising the gene editing system described herein. In some embodiments, the vectors are a viral vector. In some embodiments, the vectors are an AAV vector or a lentiviral vector. When the gene editing system comprises an AAV based gene editing vector, the vector is an AAV vector.

In one aspect, the present invention provides methods of altering the structure of a cell comprising contacting the cell with: a gene editing system described herein; or a vector described herein, under conditions that allow for alteration of the structure of the cell, thereby altering the structure of the cell. In some embodiments, the structure of the cells is altered by altering the sequence of the target nucleic acid in the cell.

In one aspect, the present invention provides methods of treating a subject by altering the structure of a cell in the subject, comprising contacting the cell with: a gene editing system described herein; or a vector described herein, under conditions that allow for alteration of the structure of the cell, thereby treating the subject by altering the structure of the cell in the subject.

In one aspect, the present invention provides methods of decreasing toxicity and/or promoting DNA repair of a break in a nucleic acid in a cell via an HDR pathway, the method comprising contacting the cell with: a gene editing system described herein; or a vector described herein, under conditions that allow for alteration of the structure of the cell, thereby treating the subject by altering the structure of the cell in the subject.

In accordance with the present invention, the cells used in the methods can be from any subject, including both human and non-human cells. In some embodiments, the cells are from a human. In some embodiments, the cells are human stem cells. In some embodiments, the cells are from a non-human subject, e.g., pig, cow, horse, cat, dog, sheep, or goat. In some embodiments, the cells are modified ex vivo.

In accordance with the present invention, TP35 inhibition can be transient, e.g., about 30 minutes, 1 hour, 2 hours, 3 hours, 4 hours, 5 hours, 6 hours, 7 hours, 8 hours, 9 hours, 10 hours, . . . up to 24 hours; or it can be more than 1 day, 2 days, 3 days, . . . up to several months. In some embodiments, cells are contacted with a TP53 inhibitor after being contacted with the nuclease (e.g., Cas9). In some embodiments, cells are contacted with a TP53 inhibitor before being contacted with the nuclease (e.g., Cas9). In some embodiments, cells are contacted with the TP53 inhibitor and the nuclease (e.g., Cas9) at the same time.

In one aspect, the present invention provides gene editing systems as described here, where the target nucleic acid is altered to comprise the sequence of at least a portion of a template nucleic acid.

In one aspect, the present invention provides gene editing systems as described herein for treatment of a subject that has a disorder that is caused by a mutation in the target nucleic acid.

In one aspect, the present invention provides gene editing systems as described herein for treating a subject that has cancer, a genetic disease, an infectious disease, a disorder caused by aberrant mitochondrial DNA (mtDNA), a metabolic disease, a disorder caused by aberrant cell cycle, a disorder caused by aberrant angiogenesis, a disorder caused by aberrant DNA damage repair, or a pain disorder.

In one aspect, the present invention provides gene editing system as described herein, or vectors as described herein, for use in methods for genetic screen, modifying a gene in a subject, or treating a disease in a subject.

In another aspect, the present invention provides methods of decreasing toxicity of gene editing to a cell by contacting said cell with an apoptosis inhibitor, e.g., a TP53 inhibitor, e.g., any TP53 inhibitor described herein.

In another aspect, provided herein are methods of modifying a donor cell or organ for transplantation. Such methods comprise contacting said donor cell or organ with an apoptosis inhibitor, e.g., any TP53 inhibitor described herein, and performing gene editing to said donor cell or organ. In some embodiments, such methods further comprise contacting the cell with one or more growth factors, e.g., basic fibroblast growth factor (bFGF). In some embodiments, the donor is a non-human subject, e.g., pig, cow, horse, cat, dog, sheep, or goat.

In some embodiments, said gene editing of the methods described above uses a nuclease, e.g., a meganuclease, zinc finger nuclease (ZFNs), transcription activator-like effector-based nuclease (TALEN), CPF1, or Cas9. In some embodiments, said gene editing of the methods described above uses a gene editing vector, e.g., a gene editing viral vector. In some embodiments, said gene editing of the methods described above comprises using of an AAV based gene editing vector, e.g., a rAAV. In some embodiments, said gene editing of the methods described above comprises using of a recombinant AAV Clade F vector.

In some embodiments, the gene editing system of the present invention is used to reduce immunological incompatibility between the donor organ and the transplant recipient and/or to reduce rejection due to viral infection of the recipient by the donor organ.

In some embodiments, the donor organ is from a pig and will be used in a xenotransplant to a human recipient, wherein the gene editing system can be used to silence porcine genes involved in hyperacute rejection, delayed xenograft rejection, cellular rejection and/or chronic rejection. For example, genes involved in hyperacute rejection, which have been successfully knocked out by a gene editing system as described herein, include α1,3-galactosyltransferase (GGTA1), cytidine monophosphate-N-acetylneuraminic acid hydroxylase (CMAH) and β1,4-N-acetyl-galactosaminyltransferase (β4GalNT2) (Petersen et al. (2016) Xenotransplantation 23(5): 338-46; Estrada et al. (2015) Xenotransplantation 22(3): 194-202).

In some embodiments, the donor organ is from a pig and will be used in a xenotransplant to a human recipient, wherein the gene editing system is used to reduce or eliminate viral transmission between the pig organ and the transplant recipient. For example, to reduce the risk of cross-species transmission of porcine endogenous retroviruses (PERVs), the gene editing system can be used to inactivate PERVs and therefore eliminate transmission of some or all PERVs to the human recipient.

Also provided herein are use of any one of the gene editing system or vector described herein in a method for genetic screen, modifying a gene in a subject, or treating a disease in a subject.

Also provided herein are use of any one of the gene editing system or vector described herein for modifying a donor cell or organ for transplantation.

### Definitions

As used in the specification and claims, the singular form “a”, “an” and “the” include plural references unless the context clearly dictates otherwise. For example, the term “a cell” includes a plurality of cells, including mixtures thereof.

As used herein, “TP53” (also known as tumor protein 53, P53; BCC7; LFS1; TRP53, terms used interchangeably) refers to either a protein encoded by the TP53 gene, and/or the TP53 gene. In human, TP53 gene is mapped to chromosomal location 17p13.1, and the human TP53 genomic sequence can be found at NG_017013.2. The mRNA and amino acid sequences of human TP53 isoforms can be found in GenBank with the following accession Nos:

NM_000546.5→NP_000537.3 cellular tumor antigen p53 isoform a;

NM_001126112.2→NP_001119584.1 cellular tumor antigen p53 isoform a;

NM_001126114.2→NP_001119586.1 cellular tumor antigen p53 isoform b;

NM_001126113.2→NP_001119585.1 cellular tumor antigen p53 isoform c;

NM_001126115.1→NP_001119587.1 cellular tumor antigen p53 isoform d;

NM_001126116.1→NP_001119588.1 cellular tumor antigen p53 isoform e;

NM_001126117.1→NP_001119589.1 cellular tumor antigen p53 isoform f;

NM_001126118.1→NP_001119590.1 cellular tumor antigen p53 isoform g;

NM_001276760.1→NP_001263689.1 cellular tumor antigen p53 isoform g;

NM_001276761.1→NP_001263690.1 cellular tumor antigen p53 isoform g;

NM_001276695.1→NP_001263624.1 cellular tumor antigen p53 isoform h;

NM_001276696.1→NP_001263625.1 cellular tumor antigen p53 isoform i;

NM_001276697.1→NP_001263626.1 cellular tumor antigen p53 isoform j;

NM_001276698.1→NP_001263627.1 cellular tumor antigen p53 isoform k;

NM_001276699.1→NP_001263628.1 cellular tumor antigen p53 isoform 1.

The term “treat” and “treatment” refer to both therapeutic treatment and prophylactic or preventive measures, wherein the object is to prevent or slow down an undesired physiological change or disorder. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The term “subject” refers to an animal, human or non-human, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats, e.g., from the metazoan Glade and has a TP53 family member, see e.g., Belyi et al., Cold Spring Harb Perspect Biol 2010; 2:a001198. Typical subjects include humans, farm animals (e.g., pig, cow, horse, sheep, or goat), and domestic pets such as cats and dogs.

An “effective amount” refers to an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A “therapeutically effective amount” of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

“Activity” of a protein refers to regulatory or biochemical functions of a protein in its native cell or tissue. Examples of activity of a protein include both direct activities and indirect activities.

The term “antibody,” as used herein, refers to a protein, or polypeptide sequence derived from an immunoglobulin molecule that specifically binds to an antigen. Antibodies can be polyclonal or monoclonal, multiple or single chain, or intact immunoglobulins, and may be derived from natural sources or from recombinant sources. Antibodies can be tetramers of immunoglobulin molecules. The term “antibody,” as used herein, also includes antibody fragments. The term “antibody fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide brudge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type Ill (Fn3)(see U.S. Pat. No. 6,703,199, which describes fibronectin polypeptide minibodies).

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, the term “RNAi agent” refer to an siRNA (short inhibitory RNA), shRNA (short or small hairpin RNA), iRNA (interference RNA) agent, RNAi (RNA interference) agent, dsRNA (double-stranded RNA), microRNA, and the like, which specifically binds to a target gene, and which mediates the targeted cleavage of another RNA transcript via an RNA-induced silencing complex (RISC) pathway.

The term “antisense oligonucleotide” refers to a single-stranded nucleic acid molecule having a nucleobase sequence that permits hybridization to a corresponding segment of a target nucleic acid.

The term “ribozyme,” as used herein, refers to a catalytic RNA molecule capable of cleaving RNA substrates. Ribozyme specificity is dependent on complementary RNA-RNA interactions.

The term “low molecular weight compound” is used to describe an organic or biological compound with a molecular weight of less than or equal to 2000 Da.

The term “gene editing vector” as used herein refers to a nucleic acid molecule that comprises a targeting element and/or an editing element. The target element is capable of recognizing a target genomic sequence. The editing element is capable of modifying the target genomic sequence, e.g., by substitution or insertion of one or more nucleotides in the genomic sequence, deletion of one or more nucleotides in the genomic sequence, alteration of genomic sequences to include regulatory sequences, insertion of transgenes at a safe harbor genomic site or other specific location in the genome, or any combination thereof. The targeting element and the editing element can be on the same nucleic acid molecule or different nucleic acid molecules. The gene editing vector can be a DNA vector, an RNA vector, a plasmid, a cosmid, or a viral vector.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1F show Cas9-dependent gene disruption is efficient and toxic to human pluripotent stem cells. FIG. 1A is a diagram showing the 2-component Cas9 system depicting all-in-one inducible Cas9 construct and lentiviral delivery of constitutive sgRNA.

FIG. 1B is a bar graph showing NGS quantification of indels at 47 sgRNA loci. sgRNA infected iCas9 cells grown in the presence of dox for 8 days. FIG. 1C is pie chart summary of efficiency and indel types generated by 47 sgRNAs. Averages shown for all 47 sgRNAs and the best sgRNA per gene. FIG. 1D is a bar graph showing Indel quantification at MAPT locus. After 10 days of dox treatment MAPT locus is completely edited. In the absence of dox, no editing was observed demonstrating Cas9 expression is tightly controlled. FIG. 1E is a set of images showing MAPT targeting sgRNA reduces colony size relative to a non-targeting control. Bright-field image of live iCas9 hPSCs cultured with dox for 3 days in the presence of a MAPT or a non-targeting sgRNA. FIG. 1F is a bar graph showing quantification of toxic response to Cas9-induced DSBs in live cells. Percent confluence was measured each day in cells expressing either the MAPT or non-targeting sgRNA grown in dox. Control reads are represented by white bars, in-frame mutations by gray bars and frameshift mutations by black bars.

FIGS. 2A-2E show CRISPR screens identify hPSC-specific toxic response to Cas9-induced DSBs. FIG. 2A is a diagram showing experimental paradigm for pooled screen in hPSCs testing 13,000 sgRNAs in 4 independent conditions H1 (parental) depicted in light gray, H1-iCas9 minus dox depicted in black, H1-iCas9 plus dox depicted in dark gray, and H1-ddCas9 plus Shield1 depicted in light gray. 2000× cells for each condition were infected with each sgRNA (0.5 MOI, 2.6*10̂7 cells). Uninfected cells were killed off with puromycin selection 24 hours after lentiviral infection. On days 4, 8, and 12 cells were dissociated, then counted to maintain 1000× representation for both DNA isolation and passaging. FIG. 2B is a bar graph showing cell counts at day 4 were reduced in Cas9 positive (plus dox or Shield1) cells relative to cells grown in the absence of Cas9 (H1, iCas9 minus dox). FIGS. 2C-2E show barcode counting of genome integrated sgRNAs via NGS to measure representation of each sgRNA. Y-axis plots Log 2(fold change) calculated for each sgRNA normalized to the initial 13,000 sgRNA library. X-axis plots each condition over time in FIGS. 2C and 2D. FIG. 2C shows fold change for the entire 13,000 sgRNA library. In the absence of Cas9 sgRNA representation does not change. In the presence of Cas9 sgRNAs both increase and decrease representation in a time-dependent manner. FIG. 2D shows fold change for 72 non-targeting control sgRNAs. In the presence of Cas9, non-targeting sgRNAs enrich their representation relative to the starting library. FIG. 2E shows hPSCs are sensitive to DSBs. X-axis plots CRISPR screens conducted in 2 hPSCs (FIG. 2D—day 12) and 14 additional transformed lines. In hPSCs non-targeting controls have a strong proliferative advantage over DSB-inducing sgRNAs and thereby increase representation throughout the course of a CRISPR screen. This response is reduced in transformed cell lines and absent in lines with TP53 mutations (underlined).

FIGS. 3A-3F show Cas9-induced DSBs trigger a TP53-dependent toxic response in hPSCs. FIG. 3A is a volcano plot depicting differential expression from RNA-seq calculated by comparing a MAPT and non-targeting control sgRNA (n=3 per condition). Adjusted p-value (padj) on y-axis, log 2(fold change) on x-axis. Each condition was cultured in the presence of dox for two days. Circles represent differentially expressed genes. FIG. 3B is a bar graph showing p21 mRNA is induced by 7 independent sgRNAs in iCas9 cells 2 days after dox treatment. Relative expression is calculated by comparing the non-targeting control (EGFP) to each targeting sgRNA. Y-axis is relative expression. X-axis RNA samples for each sgRNA. FIG. 3C is a diagram showing interactome analysis identifies TP53-dependent changes in expression caused by Cas9-induced DSBs. The 1-step TP53 hypothesis accurately explains gene expression changes for 33 out of 100 differentially expressed genes. Upregulated genes in dark gray and downregulated genes in light gray. FIG. 3D is a set of bar graphs showing TP53 is required for p21 and fas mRNA induction in response to Cas9-induced DSBs. qPCR detects an induction of p21 and fas mRNA in dox treated controls but not the TP53 mutant pool. Relative expression is calculated by comparing to untreated control cells. FIG. 3E is a set of images showing a DSB-dependent increase in TP53 and P21 protein in control cells detected by immunofluorescent staining. In the TP53 mutant pool TP53 and P21 are significantly decreased. TP53 and P21 are shown in white. DAPI co-stained nuclei are outlined in white. FIG. 3F is a line graph showing Cas9-induced toxic response is TP53-dependent. Live imaging of confluence in MAPT sgRNA expressing iCas9 cells +/− dox in control or TP53 mutant pool. Unlike dox treated control cells the TP53 mutant pool continues to grow despite the induction of DSBs.

FIGS. 4A-4D show TP53 inhibition enhances Cas9 genome engineering in hPSCs. FIG. 4A is a schematic diagram of HDR assay targeting the OCT4 locus. A dual nickase approach targeting the stop codon was used to introduce a gene trap fusing an HA tagged tdTomato to the oct4 ORF and an internal ribosome entry site (IRES) to drive the expression of the puro resistance gene off of the oct4 promoter. FIG. 4B shows TP53 inhibition increases the efficiency and yield of HDR in hPSCs. Stem cell-specific TRA-1-60 antibodies conjugated to HRP were used to visualize colonies surviving puro selection following the electroporation of OCT4 donor, dual nickases and +/− for the p53DD plasmid. In H1-hESCs and 8402-iPSCs both the number and size of colonies with precise gene targeting are increased in the presence of p53DD relative to control. FIG. 4C is dot plot showing quantification of independent replicates conducted on different weeks in both 8402-iPSCs and H1-hESCs. unpaired, two-sided t-test *p<0.05, **p<0.01. 8402-iPSCs n=3, H1-hESCs is n=2t. FIG. 4D is a set of live images of nuclear Oct4:tdTomato (white) in both control and p53DD treated hPSCs. † for the 3^{rd} replicate colonies were too large for accurate quantification.

FIGS. 5A-5E show inducible Cas9 constructs in hESCs. FIG. 5A is a diagram depiction of all-in-one dox inducible (pAAVS1-iCas9) and Shield1 inducible (pB-ddCas9 iNgn2) Cas9 constructs. Although not utilized for this manuscript the ddCas9 transgene has an all-in-one dox inducible Ngn2 that can be used for rapid generation of cortical excitatory neurons for hPSCs. TRE3G and pTRE-tight, tetracycline response element promoter, ins, insulator CAG, constitutive promoter, Tet-On 3G and rtTA16, tetracycline transactivator protein, T 2A, self-cleaving peptide, neo^{R}, neomycin resistance gene, DD, destabilizing domain, PB, piggyBac repeats, LA, left arm, RA, right arm, HSVtk, herpes simplex virus (HSV) thymidine kinase promoter. FIG. 5B shows karyotype analysis for the two clones used in the study revealed no chromosomal abnormalities when the lines were first banked. FIG. 5C is a set of images showing induction of Cas9 protein addition of dox or Shield1 increase the amount of Cas9 protein detected by immunofluorescence using an antibody to detect FLAG tagged Cas9. FIG. 5D is a bar graph showing qPCR results for cas9 mRNA, which reveals that cas9 expression is only induced in the presence of dox. FIG. 5E shows targeting of the iCas9 construct to the pAAVS1 safe harbor locus. Using a primer pair to span the AAVS1 knock-in site only amplifies in controls and indicates that iCas9 clone used in this study is homozygous. Junction PCR was used to detected both 5′ and 3′ specific junctions only in iCas9 transgenic cells.

FIGS. 6A-6C show hESCs have a toxic response to Cas9 engineering. FIG. 6A is a set of live images of iCas9 H1-hESCs cultured in the presence of a non-targeting (EGFP) control or 7 independent targeting sgRNAs demonstrates that the presence of a DSB created by targeting sgRNAs generates a toxic response resulting in reduced confluency and increases cellular debris in the media. Images were taken 24 h following their last media change to capture cellular debris suspended in the spent media. FIG. 6B is a bar graph showing very low levels of background mutations were detected in uninfected iCas9 control cells, thus confirming the specificity of the NGS assays for 47 independent sgRNAs. FIG. 6C is a bar graph showing on target MAPT indels in samples used for OFF target analysis. Quantification of indel at MAPT locus by NGS. Without dox, no indels are detected. With dox, frameshift and in-frame mutations increase overtime. Cells were infected with 1-40 ul lentivirus in 24-well plates seeded with 50,000 cells at the time of infection. Adherent samples were washed and dissociated while cellular debris was DNA isolated by spinning down the spent media prior to dissociation. All samples were void of OFF target mutations (Table S2). Control reads are represented by white bars, in-frame mutations by light gray bars and frameshift mutations by black bars.

FIGS. 7A-7B show sgRNA design flaws are consistent with DSB toxicity in hPSCs. FIG. 7A shows Log 2(fold change) for 251 sgRNAs affected by SNPs in the H1-hESC genome. In the presence of Cas9, sgRNAs with binding sites disrupted by SNPs show an increase in representation. FIG. 7B shows Log 2(fold change) for 151 sgRNAs with one or more perfect cut sites. Only in the presence of Cas9, sgRNAs with no cut sites enrich while sgRNAs with 1 or more cut sites dropout in a dose-dependent fashion.

FIGS. 8A-8D show experimental paradigm for TP53 mutant pool generation and analysis. FIG. 8A is a diagram showing locations of 3 synthetic crRNAs targeting the TP53 locus. FIG. 8B is a diagram showing experimental paradigm for TP53 mutant analysis. After recovering from mutagenesis the TP53 mutant pool and controls with an intact TP53 were infected with the MAPT lentiCRISPR. At the onset of the experiment, control and mutant pools were dissociated and plated into media with or without dox. FIG. 8C is a bar graph showing DNA from the onset of the experiment was isolated quantify mutations at the TP53 locus by NGS pipeline. No mutations are in the control pool while the mutant pool is a mix of wild-type and frameshift allele at 3 different locations. The NGS only measure one locus at a time and does not account for cis/trans mutations at other crRNA binding sites. Each mutation could be accompanied by either control reads or frameshift mutations at the other loci. The mutant pool therefore has a range from at least 50% to 93% TP53 mutant alleles. FIG. 8D is a set of bar graphs showing quantification of DAPI stained nuclei positive for TP53 or P21 protein in control and TP53 mutant pools infected with the MAPT sgRNA cultured +/− dox for two days. Dox treated controls increase the percentage of TP53 or P21 positive nuclei, and this induction is significantly reduced in the TP53 mutant pool.

FIG. 9 shows Table S1: well to well variability of 47 sgRNAs.

FIG. 10A shows Table S2a: off-target analysis (predicted off-target sites);

FIG. 10B shows Table S2b: off-target analysis (DNA samples); FIG. 10C shows Table S2c: off-target analysis (% mutations per sample).

FIG. 11 shows Table S3: top 100 differentially expressed genes.

FIG. 12 shows Table S4: primer sequences.

FIG. 13 shows Table S5: sgRNA sequences.

### DETAILED DESCRIPTION

The present invention is based, at least in part, on the findings that inhibition of TP53 reduces Cas9-induced toxicity and greatly increased the efficiency of homology dependent repair (HDR) in cells, such as human pluripotent stem cells (hPSCs). Accordingly, provided herein are methods and compositions for improviding gene editing systems (e.g., decrease toxicity and/or increase HDR efficiency) by inhibition of TP53.

### Gene Editing System

As used herein, the term “gene editing system” refers to a system comprising one or more DNA-binding domains or components and one or more DNA-modifying domains or components, or isolated nucleic acids, e.g., one or more vectors, encoding said DNA-binding and DNA-modifying domains or components. Gene editing systems are used for modifying the nucleic acid of a target gene and/or for modulating the expression of a target gene. In some embodiments, a gene editing system of the present invention further comprises an apoptosis inhibitor, e.g., a TP53 inhibitor. In some embodiments, a gene editing system of the present invention further comprises a growth factor, e.g., a basic fibroblast growth factor (bFGF). In some embodiments, a gene editing system of the present invention further comprises an apoptosis inhibitor (e.g., a TP53 inhibitor) and a growth factor (e.g., bFGF). In known gene editing systems, for example, the one or more DNA-binding domains or components are associated with the one or more DNA-modifying domains or components, such that the one or more DNA-binding domains target the one or more DNA-modifying domains or components to a specific nucleic acid site.

Certain gene editing systems are known in the art, and include but are not limited to, zinc finger nucleases, transcription activator-like effector nucleases (TALENs); clustered regularly interspaced short palindromic repeats (CRISPR)/Cas systems, meganuclease systems, and viral vector-mediated gene editing. Without wishing to be bound by theory, it is believed that the known gene editing systems may exhibit unwanted DNA-modifying activity which is detrimental to their utility in therapeutic applications. These concerns are particularly apparent in the use of gene editing systems for in vivo modification of genes or gene expression, e.g., where cells are engineered to constitutively express components of a gene editing system, such as through lentiviral or adenoviral vector transfection.

### CRISPR Gene Editing Systems

“CRISPR” as used herein refers to a set of clustered regularly interspaced short palindromic repeats, or a system comprising such a set of repeats. “Cas,” as used herein, refers to a CRISPR-associated protein. The diverse CRISPR-Cas systems can be divided into two classes according to the configuration of their effector modules: class 1 CRISPR systems utilize several Cas proteins and the crRNA to form an effector complex, whereas class 2 CRISPR systems employ a large single-component Cas protein in conjunction with crRNAs to mediate interference. One example of class 2 CRISPR-Cas system employs Cpf1 (CRISPR from Prevotella and Francisella 1). See, e.g., Zetsche et al., Cell 163:759-771 (2015), the content of which is herein incorporated by reference in its entirety. The term “Cpf1” as used herein includes all orthologs, and variants that can be used in a CRISPR system. The present invention provides compositions and methods of using TP53 inhibitors to improvide gene editing systems, including various CRISPR systems.

Naturally-occurring CRISPR systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR system has been modified for use in gene editing (silencing, enhancing or changing specific genes) in eukaryotes such as mice, primates and humans. Wiedenheft et al. (2012) Nature 482: 331-8. This is accomplished by, for example, introducing into the eukaryotic cell one or more vectors encoding a specifically engineered guide RNA (gRNA) (e.g., a gRNA comprising sequence complementary to sequence of a eukaryotic genome) and one or more appropriate RNA-guided nucleases, e.g., Cas proteins. The RNA guided nuclease forms a complex with the gRNA, which is then directed to the target DNA site by hybridization of the gRNA's sequence to complementary sequence of a eukaryotic genome, where the RNA-guided nuclease then induces a double or single-strand break in the DNA. Insertion or deletion of nucleotides at or near the strand break creates the modified genome.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

In some embodiments, the RNA-guided nuclease is a Cas molecule, e.g., a Cas9 molecule. A “Cas9 molecule,” as used herein, refers to a molecule that can interact with a gRNA molecule (e.g., sequence of a domain of a tracr) and, in concert with the gRNA molecule, localize (e.g., target or home) to a site which comprises a target sequence and PAM sequence.

According to the present invention, Cas9 molecules of, derived from, or based on the Cas9 proteins of a variety of species can be used in the methods and compositions described herein. For example, Cas9 molecules of, derived from, or based on, e.g., S. pyogenes, S. thermophilus, Staphylococcus aureus and/or Neisseria meningitidis Cas9 molecules, can be used in the systems, methods and compositions described herein. Additional Cas9 species include: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

In some embodiments, the ability of an active Cas9 molecule to interact with and cleave a target nucleic acid is PAM sequence dependent. A PAM sequence is a sequence in the target nucleic acid. In an embodiment, cleavage of the target nucleic acid occurs upstream from the PAM sequence. Active Cas9 molecules from different bacterial species can recognize different sequence motifs (e.g., PAM sequences). In an embodiment, an active Cas9 molecule of S. pyogenes recognizes the sequence motif NGG and directs cleavage of a target nucleic acid sequence 1 to 10, e.g., 3 to 5, base pairs upstream from that sequence. See, e.g., Mali et al, SCIENCE 2013; 339(6121): 823-826. In an embodiment, an active Cas9 molecule of S. thermophilus recognizes the sequence motif NGGNG and NNAG AAW (W=A or T) and directs cleavage of a core target nucleic acid sequence 1 to 10, e.g., 3 to 5, base pairs upstream from these sequences. See, e.g., Horvath et al., SCIENCE 2010; 327(5962): 167-170, and Deveau et al, J BACTERIOL 2008; 190(4): 1390-1400. In an embodiment, an active Cas9 molecule of S. mutans recognizes the sequence motif NGG or NAAR (R—A or G) and directs cleavage of a core target nucleic acid sequence 1 to 10, e.g., 3 to 5 base pairs, upstream from this sequence. See, e.g., Deveau et al., J BACTERIOL 2008; 190(4): 1390-1400.

In an embodiment, an active Cas9 molecule of S. aureus recognizes the sequence motif NNGRR (R=A or G) and directs cleavage of a target nucleic acid sequence 1 to 10, e.g., 3 to 5, base pairs upstream from that sequence. See, e.g., Ran F. et al., NATURE, vol. 520, 2015, pp. 186-191. In an embodiment, an active Cas9 molecule of N. meningitidis recognizes the sequence motif NNNNGATT and directs cleavage of a target nucleic acid sequence 1 to 10, e.g., 3 to 5, base pairs upstream from that sequence. See, e.g., Hou et al., PNAS EARLY EDITION 2013, 1-6. The ability of a Cas9 molecule to recognize a PAM sequence can be determined, e.g., using a transformation assay described in Jinek et al, SCIENCE 2012, 337:816.

Exemplary naturally occurring Cas9 molecules are described in Chylinski et al, RNA Biology 2013; 10:5, 727-737. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 1 1 bacterial family, a cluster 12 bacterial family, a cluster 13 bacterial family, a cluster 14 bacterial family, a cluster 1 bacterial family, a cluster 16 bacterial family, a cluster 17 bacterial family, a cluster 1 8 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 5 1 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. pyogenes (e.g., strain SF370, MGAS 10270, MGAS 10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA 159, NN2025), S. macacae (e.g., strain NCTC1 1558), S. gallolylicus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. cmginosus (e.g.; strain F021 1), S. agalactia* (e.g., strain NEM316, A909), Listeria monocytogenes (e.g., strain F6854), Listeria innocua (L. innocua, e.g., strain Clip 11262), Etuerococcus italicus (e.g., strain DSM 15952), or Enterococcus faecium (e.g., strain 1,23,408). Additional exemplary Cas9 molecules are a Cas9 molecule of Neisseria meningitidis (Hou et al. PNAS Early Edition 2013, 1-6) and a S. aureus Cas9 molecule.

In an embodiment, a Cas9 molecule, e.g., an active Cas9 molecule or inactive Cas9 molecule, comprises an amino acid sequence: having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; any Cas9 molecule sequence described herein or a naturally occurring Cas9 molecule sequence, e.g., a Cas9 molecule from a species listed herein or described in Chylinski et al., RNA Biology 2013, 10:5, 121-T, 1 Hou et al. PNAS Early Edition 2013, 1-6.

In an embodiment, a Cas9 molecule comprises an amino acid sequence having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; S. pyogenes Cas9 (UniProt Q99ZW2). In embodiments, the Cas9 molecule is a S. pyogenes Cas9 variant, such as a variant described in Slaymaker et al., Science Express, available online Dec. 1, 2015 at Science DOI: 10.1126/science.aad5227; Kleinstiver et al., Nature, 529, 2016, pp. 490-495, available online Jan. 6, 2016 at doi:10.1038/nature16526; or US2016/0102324, the contents of which are incorporated herein in their entirety. In an embodiment, the Cas9 molecule is catalytically inactive, e.g., dCas9. Tsai et al. (2014), Nat. Biotech. 32:569-577; U.S. Pat. Nos. 8,871,445; 8,865,406; 8,795,965; 8,771,945; and 8,697,359, the contents of which are hereby incorporated by reference in their entirety. A catalytically inactive Cas9, e.g., dCas9, molecule may be fused with a transcription modulator, e.g., a transcription repressor or transcription activator.

In an embodiment, the Cas9 molecule of the invention can be any of the Cas9 variants, including chimeric Cas9 molecules, described in, e.g., U.S. Pat. No. 8,889,356, U.S. Pat. No. 8,889,418, U.S. Pat. No. 8,932,814, WO2016022363, US20150118216, WO2014152432, US20140295556, US2016153003, U.S. Pat. No. 9,322,037, U.S. Pat. No. 9,388,430, WO2015089406, U.S. Pat. No. 9,267,135, WO2015006294, WO2016106244, WO2016057961, and WO2016131009, the content of which are hereby incorporated by reference in their entirety.

In some embodiments, the Cas9 molecule, e.g., a Cas9 of S. pyogenes, may additionally comprise one or more amino acid sequences that confer additional activity. In some aspects, the Cas9 molecule may comprise one or more nuclear localization sequences (NLSs), such as at least 1, 2, 3, 4, 5, 6, 7, 8, 9, 10, or more NLSs. Typically, an NLS consists of one or more short sequences of positively charged lysines or arginines exposed on the protein surface, but other types of NLS are known. Non-limiting examples of NLSs include an NLS sequence comprising or derived from: the NLS of the SV40 virus large T-antigen, having the amino acid sequence PKKKRKV (SEQ ID NO:1). Other suitable NLS sequences are known in the art (e.g., Sorokin, Biochemistry (Moscow) (2007) 72:13, 1439-1457; Lange J Biol Chem. (2007) 282:8, 5101-5). In any of the aforementioned embodiments, the Cas9 molecule may additionally (or alternatively) comprise a tag, e.g., a His tag, e.g., a His(6) tag or His(8) tag, e.g., at the N terminus or the C terminus.

Thus, engineered CRISPR gene editing systems, e.g., for gene editing in eukaryotic cells, typically involve (1) a guide RNA molecule (gRNA) comprising a targeting domain (which is capable of hybridizing to the genomic DNA target sequence), and sequence which is capable of binding to a Cas, e.g., Cas9 enzyme, and (2) a Cas, e.g., Cas9, protein. This second domain may comprise a domain referred to as a tracer domain. The targeting domain and the sequence which is capable of binding to a Cas, e.g., Cas9 enzyme, may be disposed on the same (sometimes referred to as a single gRNA, chimeric gRNA or sgRNA) or different molecules (sometimes referred to as a dual gRNA or dgRNA). If disposed on different molecules, each includes a hybridization domain which allows the molecules to associate, e.g., through hybridization.

gRNA molecule formats are known in the art. An exemplary gRNA molecule, e.g., dgRNA molecule, of the present invention comprises, e.g., consists of, a first nucleic acid having the sequence:
nnnnnnnnnnnnnnnnnnnnGUUUUAGAGCUAUGCUGUUUUG (SEQ ID NO: 2),
where the “n”'s refer to the residues of the targeting domain, e.g., as described herein, and may consist of 15-25 nucleotides, e.g., consists of 20 nucleotides;
and a second nucleic acid sequence having the exemplary sequence:
AACUUACCAAGGAACAGCAUAGCAAGUUAAAAUAAGGCUAGUCCGUUAUCAAC UUGAAAAAGUGGCACCGAGUCGGUGC, optionally with 1, 2, 3, 4, 5, 6, or 7 (e.g., 4 or 7, e.g., 7) additional U nucleotides at the 3′ end (SEQ ID NO: 3).

The second nucleic acid molecule may alternatively consist of a fragment of the sequence above, wherein such fragment is capable of hybridizing to the first nucleic acid. An example of such second nucleic acid molecule is:
AACAGCAUAGCAAGUUAAAAUAAGGCUAGUCCGUUAUCAACUUGAAAAAGUGG CACCGAGUCGGUGC, optionally with 1, 2, 3, 4, 5, 6, or 7 (e.g., 4 or 7, e.g., 7) additional U nucleotides at the 3′ end (SEQ ID NO:4).

Another exemplary gRNA molecule, e.g., a sgRNA molecule, of the present invention comprises, e.g., consists of a first nucleic acid having the sequence:
nnnnnnnnnnnnnnnnnnnGUUUUAGAGCUAGAAAUAGCAAGUUAAAAUAAGGCUAG UCCGUUAUCAACUUGAAAAAGUGGCACCGAGUCGGUGC (SEQ ID NO:5), where the “n”'s refer to the residues of the targeting domain, e.g., as described herein, and may consist of 15-25 nucleotides, e.g., consist of 20 nucleotides, optionally with 1, 2, 3, 4, 5, 6, or 7 (e.g., 4 or 7, e.g., 4) additional U nucleotides at the 3′ end.

Additional components and/or elements of CRISPR gene editing systems known in the art, e.g., are described in U.S. Publication No. 2014/0068797, WO2015/048577, and Cong (2013) Science 339: 819-823, the contents of which are hereby incorporated by reference in their entirety. Such systems can be generated which inhibit a target gene, by, for example, engineering a CRISPR gene editing system to include a gRNA molecule comprising a targeting domain that hybridizes to a sequence of the target gene. In embodiments, the gRNA comprises a targeting domain which is fully complementarity to 15-25 nucleotides, e.g., 20 nucleotides, of a target gene. In embodiments, the 15-25 nucleotides, e.g., 20 nucleotides, of the target gene, are disposed immediately 5′ to a protospacer adjacent motif (PAM) sequence recognized by the RNA-guided nuclease, e.g., Cas protein, of the CRISPR gene editing system (e.g., where the system comprises a S. pyogenes Cas9 protein, the PAM sequence comprises NGG, where N can be any of A, T, G or C).

In some embodiments, the gRNA molecule and RNA-guided nuclease, e.g., Cas protein, of the CRISPR gene editing system can be complexed to form a RNP complex. Such RNP complexes may be used in the methods and apparatus described herein. In other embodiments, nucleic acid encoding one or more components of the CRISPR gene editing system may be used in the methods and apparatus described herein.

In some embodiments, foreign DNA can be introduced into the cell along with the CRISPR gene editing system, e.g., DNA encoding a desired transgene, with or without a promoter active in the target cell type. Depending on the sequences of the foreign DNA and target sequence of the genome, this process can be used to integrate the foreign DNA into the genome, at or near the site targeted by the CRISPR gene editing system. For example, 3′ and 5′ sequences flanking the transgene may be included in the foreign DNA which are homologous to the gene sequence 3′ and 5′ (respectively) of the site in the genome cut by the gene editing system. Such foreign DNA molecule can be referred to “template DNA.”

In an embodiment, the CRISPR gene editing system of the present invention comprises Cas9, e.g., S. pyogenes Cas9, and a gRNA comprising a targeting domain which hybridizes to a sequence of a gene of interest. In an embodiment, the gRNA and Cas9 are complexed to form a RNP. In an embodiment, the CRISPR gene editing system comprises nucleic acid encoding a gRNA and nucleic acid encoding a Cas protein, e.g., Cas9, e.g., S. pyogenes Cas9. In an embodiment, the CRISPR gene editing system comprises a gRNA and nucleic acid encoding a Cas protein, e.g., Cas9, e.g., S. pyogenes Cas9.

In some embodiments, inducible control over Cas9, sgRNA and p53DD expression can be utilized to optimize efficiency while reducing the frequency of off-target effects thereby increasing saftey. Examples include, but are not limited to, transcriptional and post-transcriptional switches listed as follows; doxycycline inducible transcription Loew et al. (2010) BMC Biotechnol. 10:81, Shield1 inducible protein stabilization Banaszynski et al. (2016) Cell 126: 995-1004, Tamoxifen induced protein activation Davis et al. (2015) Nat. Chem. Biol. 11: 316-318, Rapamycin or optogenetic induced activation or dimerization of split Cas9 Zetsche (2015) Nature Biotechnol. 33(2): 139-142, Nihongaki et al. (2015) Nature Biotechnol. 33(7): 755-760, Polstein and Gersbach (2015) Nat. Chem. Biol. 11: 198-200, and SMASh tag drug inducible degradation Chung et al. (2015) Nat. Chem. Biol. 11: 713-720.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,697,359, 8,771,945, 8,795,965, 8,865,406, 8,871,445, 8,889,356, 8,889,418 and 8,895,308; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017). US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825). WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT7US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT US2014/041804). WO 2014/204727 (PCT US2014/041806), WO 2014/204728 (PCT/US2014/041808), and WO 2014/204729 (PCT US2014/041809). Reference is also made to US provisional patent applications 1/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836,101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT US2014/041806, each filed Jun. 10, 2014 6/10/14; PCT US2014/041808 filed Jun. 11, 2014; and PCT/US2014/62558 filed Oct. 28, 2014, and U.S. Provisional Patent Applications Ser. Nos. 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and U.S. provisional patent application 61/939,242 filed Feb. 12, 2014. Reference is made to PCT application designating, inter alia, the United States, application No. PCT/US 14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to US provisional patent application U.S. Ser. No. 61/980,012 filed Apr. 15, 2014. Reference is made to PCT application designating, inter alia, the United States, application No. PCT/US 14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. [0054] Mention is also made of U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. application 62/091,462, 12 Dec. 2014, DEAD GUIDES FOR CRISPR TRANSCRIPTION FACTORS; U.S. application 62/096,324, 23 Dec. 2014, DEAD GUIDES FOR CRISPR TRANSCRIPTION FACTORS; U.S. application 62/091,456, 12 Dec. 2014, ESCORTED AND FUNCTIONALIZED GUIDES FOR CRISPR-CAS SYSTEMS: U.S. application 62/091,461, 12 Dec. 2014, DELIVERY. USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, RNA-TARGETING SYSTEM; U.S. application 62/096,656, 24 Dec. 2014, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS: U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/054,675, 24 Sep. 2014, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTION AL-CRISPR COMPLEXES: U.S. application 62/087,475, 4 Dec. 2014, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014, MULTIFUNCTIONAL CRISPR COMPLEXES AND/OR OPTIMIZED ENZYME LINKED FUNCTIONAL-CRISPR COMPLEXES: and U.S. application 62/098,285, 30 Dec. 2014, CRISPR MEDIATED IN VIVO MODELING AND GENETIC SCREENING OF TUMOR GROWTH AND METASTASIS.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

Also with respect to general information on CRISPR-Cas Systems, mention is made of the following (also hereby incorporated herein by reference):

Multiplex genome engineering using CRISPR/Cas systems, Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);

RNA-gided editing of bacterial genomes using CRISPR-Cas systems. Jiang W., Bikard D., Cox D., Zhang F, Marraffini L A. Nat Biotechnol March; 31(3):233-9 (2013); One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013); Optical control of mammalian endogenous transcription and epigenetic states. Konermann S, Brigham M D, Trevino A E, Hsu P D, Heidenreich M, Cong L, Piatt R J, Scott D A. Church G M, Zhang F. Nature. 2013 Aug. 22; 500(7463):472-6. doi: 10.1038/Nature 12466. Epub 2013 Aug. 23;

Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5. (2013

DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);

Genome engineering using the CRISPR-Cas9 system. Ran, F A., Hsu, P D., Wright, J., Agarwala, V., Scott, D A., Zhang, F. Nature Protocols November; 8(I I):2281-308. (2013); Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang. F. Science December 12. (2013). [Epub ahead of print]; Crystal structure of cas9 in complex with guide RNA and target DNA. Nishimasu, H., Ran, F A., Hsu, P D., Konermann. S., Shehata, S I., Dohmae, N., Ishitani, R., Zhang, F., Nureki, O. Cell Feb. 27. (2014). 156(5):935-49;

Genome-wide binding of the CRISPR endonuclease Cas9 in mammalian cells. Wu X., Scott D A., Kriz A J., Chiu A C, Hsu P D., Dadon D B., Cheng A W., Trevino A E., Konermann S., Chen S., Jaenisch R., Zhang F., Sharp P A. Nat Biotechnol. (2014) April 20. doi: 10.1038/nbt.2889,

CRISPR-Cas9 Knockin Mice for Genome Editing and Cancer Modeling, Piatt et al., Cell 159(2): 440-455 (2014) DOI: 10.1016/j.cell.2014.09.014,

Development and Applications of CRISPR-Cas9 for Genome Engineering, Hsu et al. Cell 157, 1262-1278 (Jun. 5, 2014) (Hsu 2014),

Genetic screens in human cells using the CRISPR/Cas9 system, Wang et al., Science. 2014 Jan. 3; 343(6166): 80-84. doi: 10.1126/science.1246981,

Rational design of highly active sgRNAs for CRISPR-Cas9-mediated gene inactivation, Doench et al., Nature Biotechnology published online 3 Sep. 2014; doi: 10.1038/nbt.3026, and In vivo interrogation of gene function in the mammalian brain using CRISPR-Cas9, Swiech et al, Nature Biotechnology; published online 19 Oct. 2014; doi: 10.1038/nbt.3055.

Genome-scale transcriptional activation by an engineered CRISPR-Cas9 complex, onermann S, Brigham M D. Trevino A E, Joung J, Abudayyeh 00. Barcena C, Hsu P D, Habib N, Gootenberg J S, Nishimasu H, Nureki O, Zhang F., Nature. January 29; 517(7536):583-8 (2015).

A split-Cas9 architecture for inducible genome editing and transcription modulation. Zetsche B, Volz S E, Zhang F. (published online 2 Feb. 2015) Nat Biotechnol. February; 33(2): 139-42 (2015);

Genome-wide CRISPR Screen in a Mouse Model of Tumor Growth and Metastasis, Chen S, Sanjana N E, Zheng, Shalem O, Lee, Shi X, Scott D A, Song J, Pan J Q, Weissleder R, Lee H, Zhang F, Sharp P A. Cell 160, 1246-1260, Mar. 12, 2015 (multiplex screen in mouse), and

In vivo genome editing using Staphylococcus aureus Cas9, Ran F A, Cong L, Yan W X, Scott D A, Gootenberg J S, Kriz A J. Zetsche B, Shalem O, Wu X, Makarova K S, oonin E V, Sharp P A, Zhang F., (published online 1 Apr. 2015), Nature. April 9; 520(7546): 186-91 (2015).

High-throughput functional genomics using CRISPR-Cas9, Shalem et al. Nature Reviews Genetics 16, 299-311 (May 2015).

Sequence determinants of improved CRISPR sgRNA design, Xu et al., Genome Research 25, 1 147-1 157 (August 2015).

A Genome-wide CRISPR Screen in Primary Immune Cells to Dissect Regulatory Networks, Parnas et al., Cell 162, 675-686 (Jul. 30, 2015).

CRISPR/Cas9 cleavage of viral DNA efficiently suppresses hepatitis B virus, Ramanan et al., Scientific Reports 5:10833. doi: 10.1038/srepI0833 (Jun. 2, 2015).

Crystal Structure of Staphylococcus aureus Cas9, Nishimasu et al., Cell 162, 1113-1126 (Aug. 27, 2015).

BCL 11 A enhancer dissection by Cas9-mediated in situ saturating mutagenesis, Canver et al., Nature 527(7577): 192-7 (Nov. 12, 2015) doi: 10.1038/naturel 5521. Epub 2015 Sep. 16. each of which is incorporated herein by reference, and discussed briefly below:

Cong et al. engineered type II CRISPR/Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR/Cas system can be further improved to increase its efficiency and versatility.

Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems, The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.

Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.

Konermann et al. addressed the need in the art for versatile and robust technologies that enable optical and chemical modulation of DNA-binding domains based CRISPR Cas9 enzyme and also Transcriptional Activator Like Effectors.

Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity. Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.

Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.

Shaiem et al. described a new way to interrogate gene function on a genome-wide scale.

Their studies showed that delivery of a genome-scale CRISPR-Cas9 knockout (GeC O) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED 12 as well as novel hits NF2, CUL3, TADA2B, and TADAL The authors observed a high level of consistency between independent guide RNAs targeting the same gene and a high rate of hit confirmation, and thus demonstrated the promise of genome-scale screening with Cas9.

Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.

Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.

Piatt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.

Hsu et al. (2014) is a review article that discusses generally CRISPR-Cas9 history from yogurt to genome editing, including genetic screening of cells.

Wang et al, (2014) relates to a pooled, loss-of-function genetic screening approach suitable for both positive and negative selection that uses a genome-scale lentiviral single guide RNA (sgRNA) library.

Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

Swiech et al. demonstrate that AAV-mediated SpCas9 genome editing can enable reverse genetic studies of gene function in the brain.

Konermann et al. (2015) discusses the ability to attach multiple effector domains, e.g., transcriptional activator, functional and epigenomic regulators at appropriate positions on the guide such as stem or tetraloop with and without linkers.

Zetsche et al. demonstrates that the Cas9 enzyme can be split into two and hence the assembly of Cas9 for activation can be controlled.

Chen et al relates to multiplex screening by demonstrating that a genome-wide in vivo CRISPR-Cas9 screen in mice reveals genes regulating lung metastasis. >Ran et al. (2015) relates to SaCas9 and its ability to edit genomes and demonstrates that one cannot extrapolate from biochemical assays. Shalem et al. (2015) described ways in which catalytically inactive Cas9 (dCas9) fusions are used to synthetically repress (CRISPRi) or activate (CRISPRa) expression, showing, advances using Cas9 for genome-scale screens, including arrayed and pooled screens, knockout approaches that inactivate genomic loci and strategies that modulate transcriptional activity.

Shalem et al. (2015) described ways in which catalytically inactive Cas9 (dCas9) fusions are used to synthetically repress (CRISPRi) or activate (CRISPRa) expression, showing, advances using Cas9 for genome-scale screens, including arrayed and pooled screens, knockout approaches that inactivate genomic loci and strategies that modulate transcriptional activity.

Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR Cas9 knockout.

Pamas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of TIr4 signaling and previously unknown candidates were identified and classified into three functional modules with distinct effects on the canonical responses to LPS.

Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA. Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Slaymaker et al (2015) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.

Tsai et al, “Dimeric CRISPR A-guided Fokl nucleases for highly specific genome editing,” Nature Biotechnology 32(6): 569-77 (2014) which is not believed to be prior art to the instant invention or application, but which may be considered in the practice of the instant invention. Mention is also made of Konermann et al., “Genome-scale transcription activation by an engineered CRISPR-Cas9 complex,” doi:10.1038/naturel4136, incorporated herein by reference.

In general, the CRISPR-Cas or CRISPR system is as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene. a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used. In some embodiments it may be preferred in a CRISPR complex that the tracr sequence has one or more hairpins and is 30 or more nucleotides in length, 40 or more nucleotides in length. or 50 or more nucleotides in length: the guide sequence is between 10 to 30 nucleotides in length, the CRISPR/Cas enzyme is a Type II Cas9 enzyme. In embodiments of the invention the terms guide sequence and guide RNA are used interchangeably as in foregoing cited documents such as WO 2014/093622 (PCT US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and aq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10-30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MM M MMMNNNNNNNNNNNNXGG where NNN NNN NN XGG (N is A, G, T, or C; and X can be anything) has a single occurrence in the genome. A unique target sequence in a genome may include an S. pyogenes Cas9 target site of the form MMM MMMMMNNNNNNNNNNNXGG where N N NN XGG (N is A, G, T, or C; and X can be anything) has a single occurrence in the genome. For the S. thermophilus CRISPRI Cas9, a unique target sequence in a genome may include a Cas9 target site of the form MMMMMMMMNN N N NN XXAGAAW where NNN NN N XXAGAAW (N is A, G, T, or C; X can be anything; and W is A or T) has a single occurrence in the genome. A unique target sequence in a genome may include an S. thermophilus CRISPRI Cas9 target site of the form MMMMMM MN N NNN NNXXAGAAW where NNNNNNNNNNNXXAGAAW (N is A, G, T, or C; X can be anything; and W is A or T) has a single occurrence in the genome. For the S. pyogenes Cas9, a unique target sequence in a genome may include a Cas9 target site of the form MMMMMMMMNNNN NNNNNNXGGXG where NNNNNNNNNNNNXGGXG (N is A, G, T, or C; and X can be anything) has a single occurrence in the genome. A unique target sequence in a genome may include an S. pyogenes Cas9 target site of the form MMMMMMMMMNNNNNNNNNNNXGGXG where NNNNNNNNNNNXGGXG (N is A, G, T, or C; and X can be anything) has a single occurrence in the genome. In each of these sequences “ ” may be A, G, T, or C, and need not be considered in identifying a sequence as unique. In some embodiments, a guide sequence is selected to reduce the degree secondary structure within the guide sequence. In some embodiments, about or less than about 75%, 50%, 40%, 30%, 25%, 20%, 15%, 10%, 5%, 1%, or fewer of the nucleotides of the guide sequence participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1 151-62).

### TALEN Gene Editing Systems

TALENs are produced artificially by fusing a TAL effector DNA binding domain to a DNA cleavage domain. Transcription activator-like effects (TALEs) can be engineered to bind any desired DNA sequence, e.g., a target gene. By combining an engineered TALE with a DNA cleavage domain, a restriction enzyme can be produced which is specific to any desired DNA sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is, for example, a wild-type or mutated Fokl endonuclease. Several mutations to Fokl have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96.

The Fokl domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity. Miller et al. (2011) Nature Biotech. 29: 143-8.

A TALEN (or pair of TALENs) can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN, e.g., DNA encoding a transgene, and depending on the sequences of the foreign DNA and chromosomal sequence, this process can be used to integrate the transgene at or near the site targeted by the TALEN. TALENs specific to a target gene can be constructed using any method known in the art, including various schemes using modular components. Zhang et al. (2011) Nature Biotech. 29: 149-53; Geibler et al. (2011) PLoS ONE 6: e19509; U.S. Pat. No. 8,420,782; U.S. Pat. No. 8,470,973, the contents of which are hereby incorporated by reference in their entirety.

### Zinc Finger Nuclease (ZFN) Gene Editing Systems

“ZFN” or “Zinc Finger Nuclease” refer to a zinc finger nuclease, an artificial nuclease which can be used to modify, e.g., delete one or more nucleic acids of, a desired nucleic acid sequence.

Like a TALEN, a ZFN comprises a FokI nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Aced. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences.

Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases property spaced apart. Bitinaite et al. (1998) Proc. Natl. Aced. Sci. USA 95: 10570-5.

Also like a TALEN, a ZFN can create a double-stranded break in the DNA, which can create a frame-shift mutation if improperly repaired, leading to a decrease in the expression and amount of the target gene in a cell. ZFNs can also be used with homologous recombination to mutate the target gene or locus, or to introduce nucleic acid encoding a desired transgene at a site at or near the targeted sequence.

ZFNs specific to sequences in a target gene can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; and Guo et al. (2010) J. Mol. Biol. 400: 96; U.S. Patent Publication 2011/0158957; and U.S. Patent Publication 2012/0060230, the contents of which are hereby incorporated by reference in their entirety. In embodiments, The ZFN gene editing system may also comprise nucleic acid encoding one or more components of the ZFN gene editing system.

### Meganuclease Gene Editing System

“Meganuclease” refers to a meganuclease, an artificial nuclease which can be used to edit a target gene.

Meganucleases are derived from a group of nucleases which recognize 15-40 base-pair cleavage sites. Meganucleases are grouped into families based on their structural motifs which affect nuclease activity and/or DNA recognition. Members of the LAGLIDADG family are characterized by having either one or two copies of the conserved LAGLIDADG motif (see Chevalier et al. (2001), Nucleic Acids Res. 29(18): 3757-3774). The LAGLIDADG meganucleases with a single copy of the LAGLIDADG motif form homodimers, whereas members with two copies of the LAGLIDADG motif are found as monomers. The GIY-YIG family members have a GIY-YIG module, which is 70-100 residues long and includes four or five conserved sequence motifs with four invariant residues, two of which are required for activity (see Van Roey et al. (2002), Nature Struct. Biol. 9: 806-811). The His-Cys box meganucleases are characterized by a highly conserved series of histidines and cysteines over a region encompassing several hundred amino acid residues (see Chevalier et al. (2001), Nucleic Acids Res. 29(18): 3757-3774). The NHN family, the members are defined by motifs containing two pairs of conserved histidines surrounded by asparagine residues (see Chevalier et al. (2001), Nucleic Acids Res. 29(18): 3757-3774).

Strategies for engineering a meganuclease with altered DNA-binding specificity, e.g., to bind to a predetermined nucleic acid sequence are known in the art. E.g., Chevalier et al. (2002), Mol. Cell., 10:895-905; Epinat et al. (2003) Nucleic Acids Res 31: 2952-62; Silva et al. (2006) J Mol Biol 361: 744-54; Seligman et al. (2002) Nucleic Acids Res 30: 3870-9; Sussman et al. (2004) J Mol Biol 342: 31-41; Rosen et al. (2006) Nucleic Acids Res; Doyon et al. (2006) J. Am Chem Soc 128: 2477-84; Chen et al. (2009) Protein Eng Des Sel 22: 249-56; Arnould S (2006) J Mol Biol. 355: 443-58; Smith (2006) Nucleic Acids Res. 363(2): 283-94.

A meganuclease can create a double-stranded break in the DNA, which can create a frame-shift mutation if improperly repaired, e.g., via non-homologous end joining, leading to a decrease in the expression of a target gene in a cell. Alternatively, foreign DNA can be introduced into the cell along with the Meganuclease; depending on the sequences of the foreign DNA and chromosomal sequence, this process can be used to modify a target gene, e.g., correct a defect in the target gene, thus causing expression of a repaired target gene, or e.g., introduce such a defect into a wt gene, thus decreasing expression of a target gene, e.g., as described in Silva et al. (2011) Current Gene Therapy 11:11-27.

### Viral Vector-Mediated Genome Editing

In some embodiments, a vector (e.g., a viral vector or a plasmid) can be used for gene editing. For example, adeno-associated virus (‘AAV’)-mediated gene editing or genome editing have been described previously, e.g., in WO2016049230, WO2015143177, U.S. Pat. No. 7,972,856, U.S. Pat. No. 8,846,387, Khan et al., Nat Protoc. 2011 April; 6(4): 482-501; Gornalusse et al., Nat Biotechnol. 2017 August; 35(8):765-772.

AAV genome comprises a single-stranded deoxyribonucleic acid (ssDNA) that is about 4.9 kilobase long. The genome comprises inverted terminal repeats (ITRs) at both ends of the DNA strand, and two open reading frames (ORFs): rep and cap. Rep is composed of four overlapping genes encoding rep proteins required for the AAV life cycle, and cap contains overlapping nucleotide sequences of capsid proteins: VP1, VP2 and VP3, which interact together to form a capsid of an icosahedral symmetry.

The term “rAAV” refers to recombinant adeno-associated virus or recombinant AAV vector, which comprises a polynucleotide sequence not of AAV origin (i.e., a polynucleotide heterologous to AAV). The polynucleotide sequence can comprise a transgene or a sequence for genome editing. In some embodiments, the heterologous polynucleotide is flanked by at least one, and sometimes by two, AAV inverted terminal repeat (ITR) sequences. The term rAAV vector encompasses both rAAV vector particles and rAAV vector plasmids. An rAAV vector may either be single-stranded (ssAAV) or self-complementary (scAAV). An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector.” The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession NOs. NC-002077 (AAV1), AF063497 (AAV1), NC-001401 (AAV2), AF043303 (AAV2), NC-001729 (AAV3), NC-001829 (AAV4), U89790 (AAV4), NC-006152 (AAV5), AF513851 (AAV7), AF513852 (AAV8), and NC-006261 (AAV8); or in publications such as WO2005033321 (AAV1-9), WO2016049230 (AAV Clade F viruses and vectors, e.g., AAVF1-17), the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; and U.S. Pat. No. 6,156,303.

In some embodiments, genome editing may include, without limitation, substitution or insertion of one or more nucleotides in the genome, deletion of one or more nucleotides in the genome, alteration of genomic sequences including regulatory sequences, insertion of one or more nucleotides including transgenes at safe harbor sites or other specific locations in the genome, or any combination thereof. In certain embodiments, genome editing using a viral vector (e.g., an AAV vector) may result in the induction of precise alterations of one or more genomic sequences without inserting exogenous viral sequences or other footprints.

In some embodiments, the rAAV for genome editing comprises a correction genome enclosed in an AAV capsid, e.g., an AAV Clade F capsid. In some embodiments, a “correction genome” is a nucleic acid molecule that contains an editing element along with additional element(s) (e.g., a 5′ inverted terminal repeat (5′ ITR) nucleotide sequence, or a fragment thereof, and a 3′ inverted terminal repeat (3′ ITR) nucleotide sequence, or a fragment thereof) sufficient for encapsidation within a capsid as described herein. It is to be understood that the term “correction genome” does not necessarily require that an editing element contained within the correction genome will “correct” a target locus in a genome, once integrated into the target locus (e.g., correction of target locus containing a mutation by replacement with a wild-type sequence). Accordingly, in some embodiments, a correction genome may contain an editing element which may comprise a nucleotide sequence that is additive to the target locus (e.g., the target locus is the 3′ end of a first open reading frame and the editing element is a second open reading frame that, when integrated into the target locus, will create a gene that encodes a fusion protein).

In some embodiments, the rAAV for genome editing comprises a correction genome, the correction genome comprising (a) an editing element selected from an internucleotide bond or a nucleotide sequence for integration into a target locus of a mammalian chromosome, (b) a 5′ homologous arm nucleotide sequence 5′ of the editing element, having homology to a 5′ region of the mammalian chromosome relative to the target locus, and (c) a 3′ homologous arm nucleotide sequence 3′ of the editing element, having homology to a 3′ region of the mammalian chromosome relative to the target locus. In some embodiments, the rAAV comprises a correction genome, the correction genome comprising an editing element nucleotide sequence for integration into a target locus of a mammalian chromosome, the correction genome having an essential absence of a promoter operatively linked to the editing element nucleotide sequence. In some embodiments, the rAAV comprises a correction genome, the correction genome comprising an editing element selected from an internucleotide bond or a nucleotide sequence for integration into a target locus of a mammalian chromosome in a cell; the AAV having a chromosomal integration efficiency of at least about 1% (e.g., at least about 2%, at least about 5%, at least about 10%, at least about 20%, at least about 30%, at least about 40%, at least about 50%, at least about 60%, at least about 70%, at least about 80%, or at least about 90%) for integrating the editing element into the target locus of the mammalian chromosome in the cell. In some embodiments, the rAAV comprises a correction genome, the correction genome comprising an editing element selected from an internucleotide bond or a nucleotide sequence for integration into a target locus of a mammalian chromosome in a cell; the AAV having a chromosomal integration efficiency of at least about 1% (e.g., at least about 2%, at least about 5%, at least about 10%, at least about 20%, at least about 30%, at least about 40%, at least about 50%, at least about 60%, at least about 70%, at least about 80%, or at least about 90%) in the absence of an exogenous nuclease for integrating the editing element into the target locus of the mammalian chromosome in the cell. In some embodiments of any one of the correction genomes, the correction genome has an essential absence of a promoter operatively linked to the editing element nucleotide sequence. In some embodiments of any one of the correction genomes, the correction genome further comprises an exogenous promoter operatively linked to the editing element. In some embodiments, the rAAV has a chromosomal integration efficiency of at least about 1%, at least about 2%, at least about 3%, at least about 4%, at least about 5%, at least about 10%, at least about 20%, at least about 30%, at least about 40%, at least about 50%, at least about 60%, at least about 70%, at least about 80%, or at least about 90% for integrating an editing element into a target locus of a mammalian chromosome in a cell.

A correction genome as described herein can comprise a 5′ inverted terminal repeat (5′ ITR) nucleotide sequence 5′ of the 5′ homologous arm nucleotide sequence, and a 3′ inverted terminal repeat (3′ ITR) nucleotide sequence 3′ of the 3′ homologous arm nucleotide sequence. In some embodiments, the 5′ ITR nucleotide sequence and the 3′ ITR nucleotide sequence are substantially identical (e.g., at least 90%, at least 95%, at least 98%, at least 99% identical or 100% identical) to an AAV2 virus 5′ ITR and an AAV2 virus 3′ ITR, respectively. In some embodiments, the 5′ ITR nucleotide sequence has at least 95% (e.g., at least 96%, at least 97%, at least 98%, at least 99%, or 100%) sequence identity to SEQ ID NO: 11, and the 3′ ITR nucleotide sequence has at least 95% (e.g., at least 96%, at least 97%, at least 98%, at least 99%, or 100%) sequence identity to SEQ ID NO: 12. In some embodiments, the 5′ ITR nucleotide sequence and the 3′ ITR nucleotide sequence are substantially identical (e.g., at least 90%, at least 95%, at least 98%, at least 99% identical or 100% identical) to an AAV5 virus 5′ ITR and an AAV5 virus 3′ ITR, respectively. In some embodiments, the 5′ ITR nucleotide sequence has at least 95% (e.g., at least 96%, at least 97%, at least 98%, at least 99%, or 100%) sequence identity to SEQ ID NO: 13, and the 3′ ITR nucleotide sequence has at least 95% (e.g., at least 96%, at least 97%, at least 98%, at least 99%, or 100%) sequence identity to SEQ ID NO: 14. In some embodiments, the 5′ ITR nucleotide sequence and the 3′ ITR nucleotide sequence are substantially mirror images of each other (e.g., are mirror images of each other except for at 1, 2, 3, 4 or 5 nucleotide positions in the 5′ or 3′ ITR).

Exemplary AAV2 5′ ITR
(SEQ ID NO: 11)
ttggccactccctctctgcgcgctcgctcgctcactgaggccgggcgacc
aaaggtcgcccgacgcccgggctttgcccgggcggcctcagtgagcgagc
gagcgcgcagagagggagtggccaactccatcactaggggttcct
Exemplary AAV2 3′ ITR
(SEQ ID NO: 12)
aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgcagagagggagtggccaa
Exemplary AAV5 5′ ITR
(SEQ ID NO: 13)
ctctcccccctgtcgcgttcgctcgctcgctggctcgtttgggggggtgg
cagctcaaagagctgccagacgacggccctctggccgtcgcccccccaaa
cgagccagcgagcgagcgaacgcgacaggggggagagtgccacactctca
agcaagggggttttgta
Exemplary AAV5 3′ ITR
(SEQ ID NO: 14)
tacaaaacctccttgcttgagagtgtggcactctcccccctgtcgcgttc
gctcgctcgctggctcgtttgggggggtggcagctcaaagagctgccaga
cgacggccctctggccgtcgcccccccaaacgagccagcgagcgagcgaa
cgcgacaggggggagag

In some embodiments, a correction genome as described herein is no more than 7 kb (kilobases), no more than 6 kb, no more than 5 kb, or no more than 4 kb in size. In some embodiments, a correction genome as described herein is between 4 kb and 7 kb, 4 kb and 6 kb, 4 kb and 5 kb, or 4. Ikb and 4.9 kb.

In some embodiments, the rAAV is an AAV Clade F vector, e.g., the rAAV comprises one or more Clade F AAV capsids or capsid variants. Clade F AAV vectors include AAV9 and AAVHSC1-17 (or AAVF1-17), which has been described in U.S. Pat. No. 8,628,966, U.S. Pat. No. 8,927,514, WO2016049230; and Smith & Chatterjee et al., Molecular Therapy 22 (9): 1625-1634, 2014, the disclosures of which are incorporated by reference herein. In certain embodiments, a donor vector may be packaged into the Clade F capsids or capsid variants according to a standard AAV packaging method resulting in formation of the AAV Clade F vector or AAV vector variant (see e.g., Chatterjee, 1992, Science 258, 1485-1488).

### TP53 Inhibitors

TP53 (or p53) is a transcription factor of about 53 KDa, which regulates the cell cycle and functions as a tumor suppressor. TP53 contains a transcriptional activation domain, a DNA binding domain, and an oligomerization domain. TP53 plays an important role in providing stability by preventing genome mutation. TP53 protein responds to diverse cellular stresses to regulate expression of target genes, thereby inducing cell cycle arrest, apoptosis, senescence, DNA repair, or changes in metabolism. Mutations in TP53 gene are associated with a variety of human cancers, including hereditary cancers such as Li-Fraumeni syndrome.

In normal cells, TP53 is generally held in an inactive form, bound to the protein MDM2 (HDM2 in humans), which prevents TP53 activity and promotes TP53 degradation by acting as a ubiquitin ligase. As such, activating MDM2 in any manner promotes the degradation of TP53 and is inhibit its function. Active TP53 is induced in response to various cancer-causing agents such as UV radiation, oncogenes, and some DNA-damaging drugs. DNA damage is sensed by “checkpoints” in a cell's cycle, and causes proteins such as ATM, CHK1 and CHK2 to phosphorylate TP53 at sites that are close to or within the MDM2-binding region and p300-binding region of the protein. Oncogenes also stimulate TP53 activation, mediated by the protein p14ARF. Some oncogenes can also stimulate the transcription of proteins which bind to MDM2 and inhibit its activity. Once activated, TP53 activates expression of several genes including one encoding for p21, a cell cycle inhibitor. p21 binds to G1-S-phase and S-phase cyclin CDK complexes inhibiting their activity. See, e.g., Mills, Genes & Development, 19: 2091-2099 (2005).

Provided herein are methods of decreasing toxicity and/or increasing gene editing efficiency (e.g., increasing homology dependent repair, “HDR”) of a gene editing system in cells, such as human cells, including pluripotent stem cells by introducing into the cells an agent that inhibits TP53.

Examples of agents that inhibit the function of TP53 protein include, but are not limited to, a TP53 variant comprising one or more mutations that can inhibit naturally occurring TP53 expression (referred herein as “TP53 variant”) or a nucleic acid that encodes such a TP53 variant, a chemical inhibitor of TP53 or TP53 pathway, an aptamer, an antisense oligonucleotide, a RNAi agent, a ribozyme, an anti-TP53 neutralizing or antagonist antibody or derivative or a nucleic acid that encodes such antibody or derivative, a decoy nucleic acid comprising a consensus sequence of a p53-responsive element, or a gene editing system that target TP53 to modify it's expression, for example, a dCas9-transcription repressor fusion molecule can be used to target TP53 or its regulatory elements to inhibit TP53 expression/function. At least some TP53 inhibitors are commercially available, such as nutlin-3; pifithrin-alpha hydrobromide; roscovitine; pifithrin-alpha, p-Nitro; pifithrin-mu; 9-hydroxyellipticine, hydrochloride; pifithrin-alpha, p-nitro, cyclic; cyclic pifithrin-alpha hydrobromide; SJ 1725550; (+)-nutlin-3; (ndas)-nutlin-3; and ReAcp53.

Other TP53 inhibitors include chemical or genetic activators of MDM2 or a related ubiquitn ligase that promotes the degradation of TP53. Such TP53 inhibitors can be a recombinant MDM2 protein or variant thereof, or a nucleic acid encoding such MDM2 protein or variant, which can degrade TP53 protein and therefore inhibit p53 cellular function. In some embodiments, TP53 inhibitor can be a noncleavable MDM2 variant or a nucleic acid encoding a noncleavable MDM2 variant. For example, TP53 inhibitor can be a MDM2 variant that is resistant to Caspase 2 cleavage or a nucleic acid encoding such a MDM2 variant, e.g., a MDM2 variant containing an amino acid substitution or deletion of Asp 367 (see Oliver et al., Mol Cell. 2011 Jul. 8; 43(1):57-71). In some embodiments, TP53 inhibitor can be a hyperactive MDM2 variant or a nucleic acid encoding a hyperactive MDM2 variant. For example, TP53 inhibitor can be a MDM2-3AD construct containing two extra tandem copies of the acidic domain (AD) sequence (residues 221 to 280) as described in Cheng et al., Mol Cell Biol. 2014 August; 34(15): 2800-2810). Other hyperactive MDM2 include MDM2-S395A or MDM2-S294A, see Li et al., Cancer Cell. 2012 May 25; 21(5): 668-679.

In some embodiments, the TP53 inhibitor is a recombinant MDM2 protein or variant having an amino acid sequence selected from any one of the following sequences:

MDM2-S395
(SEQ ID NO: 15)
MCNTNMSVPTDGAVTTSQIPASEQETLVRPKPLLLKLLKSVGAQKDTYTM
KEVLFYLGQYIMTKRLYDEKQQHIVYCSNDLLGDLFGVPSFSVKEHRKIY
TMIYRNLVVVNQQESSDSGTSVSENRCHLEGGSDQKDLVQELQEEKPSSS
HLVSRPSTSSRRRAISETEENSDELSGERQRKRHKSDSISLSFDESLALC
VIREICCERSSSSESTGTPSNPDLDAGVSEHSGDWLDQDSVSDQFSVEFE
VESLDSEDYSLSEEGQELSDEDDEVYQVTVYQAGESDTDSFEEDPEISLA
DYWKCTSCNEMNPPLPSHCNRCWALRENWLPEDKGKDKGEISEKAKLENS
TQAEEGFDVPDCKKTIVNDSRESCVEENDDKITQASQSQESEDYSQPSTS
SSIIYSSQEDVKEFEREETQDKEESVESSLPLNAIEPCVICQGRPKNGCI
VHGKTGHLMACFTCAKKLKKRNKPCPVCRQPIQMIVLTFP
MDM2-5395A
(SEQ ID NO: 16)
MCNTNMSVPTDGAVTTSQIPASEQETLVRPKPLLLKLLKSVGAQKDTYTM
KEVLFYLGQYIMTKRLYDEKQQHIVYCSNDLLGDLFGVPSFSVKEHRKIY
TMIYRNLVVVNQQESSDSGTSVSENRCHLEGGSDQKDLVQELQEEKPSSS
HLVSRPSTSSRRRAISETEENSDELSGERQRKRHKSDSISLSFDESLALC
VIREICCERSSSSESTGTPSNPDLDAGVSEHSGDWLDQDSVSDQFSVEFE
VESLDSEDYSLSEEGQELSDEDDEVYQVTVYQAGESDTDSFEEDPEISLA
DYWKCTSCNEMNPPLPSHCNRCWALRENWLPEDKGKDKGEISEKAKLENS
TQAEEGFDVPDCKKTIVNDSRESCVEENDDKITQASQSQESEDYAQPSTS
SSIIYSSQEDVKEFEREETQDKEESVESSLPLNAIEPCVICQGRPKNGCI
VHGKTGHLMACFTCAKKLKKRNKPCPVCRQPIQMIVLTYFP
MDM2-S294
(SEQ ID NO: 17)
MIYRNLVVVNQQESSDSGTSVSENRCHLEGGSDQKDLVQELQEEKPSSSH
LVSRPSTSSRRRAISETEENSDELSGERQRKRHKSDSISLSFDESLALCV
IREICCERSSSSESTGTPSNPDLDAGVSEHSGDWLDQDSVSDQFSVEFEV
ESLDSEDYSLSEEGQELSDEDDEVYQVTVYQAGESDTDSFEEDPEISLAD
YWKCTSCNEMNPPLPSHCNRCWALRENWLPEDKGKDKGEISEKAKLENST
QAEEGFDVPDCKKTIVNDSRESCVEENDDKITQASQSQESEDYSQPSTSS
SIIYSSQEDVKEFEREETQDKEESVESSLPLNAIEPCVICQGRPKNGCIV
HGKTGHLMACFTCAKKLKKRNKPCPVCRQPIQMIVLTYFP
MDM2-S294A
(SEQ ID NO: 18)
MIYRNLVVVNQQESSDSGTSVSENRCHLEGGSDQKDLVQELQEEKPSSSH
LVSRPSTSSRRRAISETEENSDELSGERQRKRHKSDSISLSFDESLALCV
IREICCERSSSSESTGTPSNPDLDAGVSEHSGDWLDQDSVSDQFSVEFEV
ESLDSEDYSLSEEGQELSDEDDEVYQVTVYQAGESDTDSFEEDPEISLAD
YWKCTSCNEMNPPLPSHCNRCWALRENWLPEDKGKDKGEISEKAKLENST
QAEEGFDVPDCKKTIVNDSRESCVEENDDKITQASQSQESEDYAQPSTSS
SIIYSSQEDVKEFEREETQDKEESVESSLPLNAIEPCVICQGRPKNGCIV
HGKTGHLMACFTCAKKLKKRNKPCPVCRQPIQMIVLTYFP.

In some embodiments, TP53 inhibitor can be a MDM2 variant or TP53 variant that enhances the interaction between TP53 and MDM2, or a nucleic acid encoding such MDM2 or TP53 variant. Such TP53 inhibitors include MDM2 variants having higher binding affinity for TP53 than wild type MDM2, or TP53 variants having higher binding affinity for MDM2 than wild type TP53. For example, TP53 variant with P27A mutation, or P12A, P13A, P27A triple mutations have been shown to bind to MDM2 at higher affinity than wild type TP53 (Borcherds et al., Nature Chemical Biology 10, 1000-1002, 2014).

### TP53 Variants and Nucleic Acids Encoding Such Variants

The agent that inhibits TP53 can be a TP53 variant comprising one or more mutations that can inhibit naturally occurring TP53 expression, or a nucleic acid encoding such a variant. The choice of a TP53 variant according to the present invention is not particularly limited, as far as the mutant is capable of competitively acting against the naturally occuring TP53 protein endogenously expressed in a subject's (e.g., human) cells to inhibit the function thereof. For example, a TP53 variant according to the present invention can be TP53P278S (a point mutation of the proline at the position 278. Corresponding variant in mouse is TP53P275S) located in the DNA-binding region of TP53, see de Vries, A, Proc. Natl. Acad. Sci. USA, 99, 2948-2953, 2002; TP53DD (or p53DD), deletion of amino acids at the positions 11-304 (in mouse, corresponds to the deletion of the amino acids at the positions 14-301), see Bowman, T., Genes Develop., 10, 826-835, 1996. Other known TP53 variants include, for example, TP53S61A (TP53S58A in mouse, resulting from a point mutation of the serine at the position 58 of mouse TP53 (in the case of humans, position 61) to alanine; TP53C135Y (TP53C132Y in mouse), resulting from a point mutation of the cysteine at the position 135 of human p53 (in the case of mouse, position 132) to tyrosine; TP53A138V (TP53A135V in mouse, resulting from a point mutation of the alanine at the position 135 of mouse TP53 (in the case of humans, position 138) to valine); TP53R175H (TP53R172H in mouse, resulting from a point mutation of the arginine at the position 172 (in the case of humans, position 175) to histidine); TP53R273H (TP53R270H in mouse, resulting from a point mutation of the arginine at the position 270 (in the case of humans, position 273) to histidine); TP53D281N (TP53D278N in mouse, resulting from point mutation of the aspartic acid at the position 278 of mouse TP53 (in the case of humans, position 281) to asparagine).

In some embodiments, a TP53 variant of the present invention is TP53DD. For example, a TP53DD can have an amino acid sequence selected from any one of the following sequences:

(SEQ ID NO: 6)
MEEPQSDPSVEPPLSTKRALPNNTSSSPQPKKKPLDGEYFTLQIRGRERF
EMFRELNEALELKDAQAGKEPGGS RAHSSHLKSKKGQSTSRHKKLMFKT
EGPDSD;
(SEQ ID NO: 7)
MASMTGGQQMGSPPGSTKRALPNNTSSSPQPKKKPLDGEYFTLQIRGRER
FEMFRELNEALELKDAQAGKEPGGSRAHSSHLKSKKGQSTSRHKKHMFKT
EGPDSD;
(SEQ ID NO: 8)
MTAMEESQSDISLKRALPTCTSASPPQKKKPLDGEYFTLKIRGRKRFEMF
RELNEALELKDAHATEESGDSRAHSSYLKTKKGQSTSRHKKTMVKKVGPD
SD*.

A TP53 variant of the present invention can be obtained by, for example, the technique described below. First, an appropriate oligonucleotide is synthesized as a probe or primer on the basis of the mouse or human TP53 cDNA sequence, and a mouse or human TP53 cDNA is cloned from a mRNA, cDNA or cDNA library derived from a mouse or human cell or tissue, using the hybridization method or the (RT-)PCR method, and is subcloned into an appropriate plasmid. In a form wherein a codon of the site into which a mutation is to be introduced, a primer comprising the site is synthesized, and inverse PCR is performed using this primer with the plasmid incorporating the TP53 cDNA as a template, whereby a nucleic acid that encodes the desired TP53 variant is acquired. In the case of a deletion mutant like p53DD, a primer may be designed outside the site to be deleted, and inverse PCR may be performed as described above. By introducing the thus-obtained nucleic acid that encodes the TP53 variant of the invention into a host cell, and recovering a recombinant protein from the cultured cell or its conditioned medium, the desired TP53 variants of the invention can be acquired. Examples of TP53 variants can be found in, e.g., Rivlin et al., 2011, Genes Cancer 2(4):466-474, the content of which is incorporated by reference herein in its entirety.

Although permanent inhibition of TP53 function potentially increases the risk of carcinogenesis, because a TP53 variant of the invention undergoes degradation by protease and disappears gradually in the transfected cell, and correspondingly the function of TP53 endogenously expressed in the cell is restored, use of a TP53 variant protein can be suitable in cases where high safety is required as in the utilization of the edited cells for therapeutic purposes.

In some embodiments, the agent that inhibits TP53 function is a nucleic acid that encodes a TP53 variant of the invention. The nucleic acid may be a DNA or an RNA, or a DNA/RNA chimera, and is preferably a DNA. The nucleic acid may be double-stranded or single-stranded. A cDNA that encodes a TP53 variant of the invention can be cloned by the techniques known in the art. The cDNA can be inserted into an appropriate expression vector and transferred to cells.

For example, a nucleic acid encoding TP53DD can have the following nucleotide sequence:

(SEQ ID NO: 9)
atgactgccatggaggagtcacagtcggatatcagcctcaagagagcgct
gcccacctgcacaagcgcctctcccccgcaaaagaaaaaaccacttgatg
gagagtatttcaccctcaagatccgcgggcgtaaacgcttcgagatgttc
cgggagctgaatgaggccttagagttaaaggatgcccatgctacagagga
gtctggagacagcagggctcactccagctacctgaagaccaagaagggcc
agtctacttcccgccataaaaaaacaatggtcaagaaagtggggcctgac
tcagactga.

TP53 inhibitor can also be a fusion protein, e.g., a Cas9 fusion protein, e.g., a Cas9 fused to a heterologous effector domain that inhibits TP53. In some embodiments, such a TP53 inhibitor is a Cas9 fused to a dominant negative TP53, or a Cas9 fused to MDM2 or variant. For example, such fusion protein can have an amino acid sequence selected from any one of the following sequences:

CAS9: MDM2
(SEQ ID NO: 19)
MAPKKKRKVDKKYSIGLAIGTNSVGWAVITDEYKVPSKKFKVLGNTDRHS
IKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKL
VDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELG
SQILKEHPVENTQLQNEKLYLYYLQNGRDMYVDQELDINRLSDYDVDAIV
PQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTK
YDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKK
DLIIKLPKYSLFELENGRKRMLASAGELQKGNELALPSKYVNFLYLASHY
EKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEV
LDATLIHQSITGLYETRIDLSQLGGDSRADPKKKRKDAKSLTAWSRTLVT
FKDVFVDFTREEWKLLDTAQQIVYRNVMLENYKNLVSLGYQLTKPDVILR
LEKGEEPWLVSLGSGSPAAKRVKLDEDPAAKRVKLDLIKTSGSGMCNTNM
SVPTDGAVTTSQIPASEQETLVRPKPLLLKLLKSVGAQKDTYTMKEVLFY
LGQYIMTKRLYDEKQQHIVYCSNDLLGDLFGVPSFSVKEHRKIYTMIYRN
LVVVNQQESSDSGTSVSENRCHLEGGSDQKDLVQELQEEKPSSSHLVSRP
STSSRRRAISETEENSDELSGERQRKRHKSDSISLSFDESLALCVIREIC
CERSSSSESTGTPSNPDLDAGVSEHSGDWLDQDSVSDQFSVEFEVESLDS
EDYSLSEEGQELSDEDDEVYQVTVYQAGESDTDSFEEDPEISLADYWKCT
SCNEMNPPLPSHCNRCWALRENWLPEDKGKDKGEISEKAKLENSTQAEEG
FDVPDCKKTIVNDSRESCVEENDDKITQASQSQESEDYSQPSTSSSIIYS
SQEDVKEFEREETQDKEESVESSLPLNAIEPCVICQGRPKNGCIVHGKTG
HLMACFTCAKKLKKRNKPCPVCRQPIQMIVLTYFP
CAS9: MDM2(S395A)
(SEQ ID NO: 20)
MAPKKKRKVDKKYSIGLAIGTNSVGWAVITDEYKVPSKKFKVLGNTDRHS
IKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKL
VDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELG
SQILKEHPVENTQLQNEKLYLYYLQNGRDMYVDQELDINRLSDYDVDAIV
PQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTK
YDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKK
DLIIKLPKYSLFELENGRKRMLASAGELQKGNELALPSKYVNFLYLASHY
EKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEV
LDATLIHQSITGLYETRIDLSQLGGDSRADPKKKRKDAKSLTAWSRTLVT
FKDVFVDFTREEWKLLDTAQQIVYRNVMLENYKNLVSLGYQLTKPDVILR
LEKGEEPWLVSLGSGSPAAKRVKLDEDPAAKRVKLDLIKTSGSGMCNTNM
SVPTDGAVTTSQIPASEQETLVRPKPLLLKLLKSVGAQKDTYTMKEVLFY
LGQYIMTKRLYDEKQQHIVYCSNDLLGDLFGVPSFSVKEHRKIYTMIYRN
LVVVNQQESSDSGTSVSENRCHLEGGSDQKDLVQELQEEKPSSSHLVSRP
STSSRRRAISETEENSDELSGERQRKRHKSDSISLSFDESLALCVIREIC
CERSSSSESTGTPSNPDLDAGVSEHSGDWLDQDSVSDQFSVEFEVESLDS
EDYSLSEEGQELSDEDDEVYQVTVYQAGESDTDSFEEDPEISLADYWKCT
SCNEMNPPLPSHCNRCWALRENWLPEDKGKDKGEISEKAKLENSTQAEEG
FDVPDCKKTIVNDSRESCVEENDDKITQASQSQESEDYAQPSTSSSIIYS
SQEDVKEFEREETQDKEESVESSLPLNAIEPCVICQGRPKNGCIVHGKTG
HLMACFTCAKKLKKRNKPCPVCRQPIQMIVLTYFP
CAS9: MDM2(short)
(SEQ ID NO: 21)
MAPKKKRKVDKKYSIGLAIGTNSVGWAVITDEYKVPSKKFKVLGNTDRHS
IKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKL
VDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELG
SQILKEHPVENTQLQNEKLYLYYLQNGRDMYVDQELDINRLSDYDVDAIV
PQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTK
YDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKK
DLIIKLPKYSLFELENGRKRMLASAGELQKGNELALPSKYVNFLYLASHY
EKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEV
LDATLIHQSITGLYETRIDLSQLGGDSRADPKKKRKDAKSLTAWSRTLVT
FKDVFVDFTREEWKLLDTAQQIVYRNVMLENYKNLVSLGYQLTKPDVILR
LEKGEEPWLVSLGSGSPAAKRVKLDEDPAAKRVKLDLIKTSGSGMIYRNL
VVVNQQESSDSGTSVSENRCHLEGGSDQKDLVQELQEEKPSSSHLVSRPS
TSSRRRAISETEENSDELSGERQRKRHKSDSISLSFDESLALCVIREICC
ERSSSSESTGTPSNPDLDAGVSEHSGDWLDQDSVSDQFSVEFEVESLDSE
DYSLSEEGQELSDEDDEVYQVTVYQAGESDTDSFEEDPEISLADYWKCTS
CNEMNPPLPSHCNRCWALRENWLPEDKGKDKGEISEKAKLENSTQAEEGF
DVPDCKKTIVNDSRESCVEENDDKITQASQSQESEDYSQPSTSSSIIYSS
QEDVKEFEREETQDKEESVESSLPLNAIEPCVICQGRPKNGCIVHGKTGH
LMACFTCAKKLKKRNKPCPVCRQPIQMIVLTYFP
CAS9: MDM2(short)S395A
(SEQ ID NO: 22)
MAPKKKRKVDKKYSIGLAIGTNSVGWAVITDEYKVPSKKFKVLGNTDRHS
IKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKL
VDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELG
SQILKEHPVENTQLQNEKLYLYYLQNGRDMYVDQELDINRLSDYDVDAIV
PQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTK
YDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKK
DLIIKLPKYSLFELENGRKRMLASAGELQKGNELALPSKYVNFLYLASHY
EKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEV
LDATLIHQSITGLYETRIDLSQLGGDSRADPKKKRKDAKSLTAWSRTLVT
FKDVFVDFTREEWKLLDTAQQIVYRNVMLENYKNLVSLGYQLTKPDVILR
LEKGEEPWLVSLGSGSPAAKRVKLDEDPAAKRVKLDLIKTSGSGMIYRNL
VVVNQQESSDSGTSVSENRCHLEGGSDQKDLVQELQEEKPSSSHLVSRPS
TSSRRRAISETEENSDELSGERQRKRHKSDSISLSFDESLALCVIREICC
ERSSSSESTGTPSNPDLDAGVSEHSGDWLDQDSVSDQFSVEFEVESLDSE
DYSLSEEGQELSDEDDEVYQVTVYQAGESDTDSFEEDPEISLADYWKCTS
CNEMNPPLPSHCNRCWALRENWLPEDKGKDKGEISEKAKLENSTQAEEGF
DVPDCKKTIVNDSRESCVEENDDKITQASQSQESEDYAQPSTSSSIIYSS
QEDVKEFEREETQDKEESVESSLPLNAIEPCVICQGRPKNGCIVHGKTGH
LMACFTCAKKLKKRNKPCPVCRQPIQMIVLTYFP
Cas9: mp53DD
(SEQ ID NO: 23)
MAPKKKRKVDKKYSIGLAIGTNSVGWAVITDEYKVPSKKFKVLGNTDRHS
IKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKL
VDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELG
SQILKEHPVENTQLQNEKLYLYYLQNGRDMYVDQELDINRLSDYDVDAIV
PQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTK
YDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKK
DLIIKLPKYSLFELENGRKRMLASAGELQKGNELALPSKYVNFLYLASHY
EKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEV
LDATLIHQSITGLYETRIDLSQLGGDSRADPKKKRKDAKSLTAWSRTLVT
FKDVFVDFTREEWKLLDTAQQIVYRNVMLENYKNLVSLGYQLTKPDVILR
LEKGEEPWLVSLGSGSPAAKRVKLDEDPAAKRVKLDLIKTSGSGMTAMEE
SQSDISLKRALPTCTSASPPQKKKPLDGEYFTLKIRGRKRFEMFRELNEA
LELKDAHATEESGDSRAHSSYLKTKKGQSTSRHKKTMVKKVGPDSD
Cas9: hp53DD
(SEQ ID NO: 24)
MAPKKKRKVDKKYSIGLAIGTNSVGWAVITDEYKVPSKKFKVLGNTDRHS
IKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKL
VDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELG
SQILKEHPVENTQLQNEKLYLYYLQNGRDMYVDQELDINRLSDYDVDAIV
PQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTK
YDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKK
DLIIKLPKYSLFELENGRKRMLASAGELQKGNELALPSKYVNFLYLASHY
EKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEV
LDATLIHQSITGLYETRIDLSQLGGDSRADPKKKRKDAKSLTAWSRTLVT
FKDVFVDFTREEWKLLDTAQQIVYRNVMLENYKNLVSLGYQLTKPDVILR
LEKGEEPWLVSLGSGSPAAKRVKLDEDPAAKRVKLDLIKTSGSGMEEPQS
DPSVEPPLSTKRALPNNTSSSPQPKKKPLDGEYFTLQIRGRERFEMFREL
NEALELKDAQAGKEPGGSRAHSSHLKSKKGQSTSRHKKLMFKTEGPDSD.

### Low Molecular Weight Compounds

The agent that inhibits TP53 can be a low molecular weight compound, e.g., a compound with a molecular weight of less than or equal to 2000 Da. For example, such an agent can be a chemical inhibitor of TP53, e.g., pifithrin (PFT)-α and -β, which are described in WO00/44364, PFT-μ disclosed in Storm et al. (Nat. Chem. Biol. 2, 474 (2006)), analogue thereof and salts thereof (e.g., acid addition salts such as hydrochlorides and hydrobromides, and the like). Of these, PFT-α and analogues thereof [2-(2-Imino-4,5,6,7-tetrahydrobenzothiazol-3-yl)-1-p-tolylethanone, HBr (product name: Pifithrin-α) and 1-(4-Nitrophenyl)-2-(4,5,6,7-tetrahydro-2-imino-β(2H)-benzothiazolyl)ethanone, HBr (product name: Pifithrin-α, p-Nitro)], PFT-β and analogues thereof [2-(4-Methylphenyl)imidazo[2,1-b]-5,6,7,8-tetrahydrobenzothiazole, HBr (product name: Pifithrin-α, Cyclic) and 2-(4-Nitrophenyl)imidazo[2,1-b]-5,6,7,8-tetrahydrobenzothiazole (product name: Pifithrin-α, p-Nitro, Cyclic)], and PFT-μ [Phenylacetylenylsulfonamide (product name: Pifithrin-μ)] are commercially available from Merck.

The agent that inhibits TP53 can also be a chemical inhibitor of an upstream signal cascade that leads to TP53 activation or a downstream signal cascade mediated by activated TP53. In another word, an agent that inhibits TP53 can be a TP53 pathway inhibitor. For example, TP53 inhibitor can be a substance that inhibits the expression or function (Myc inhibitory activity) of p21, whose transcription is activated by TP53. A p53 pathway inhibitor can also be a substance that inhibits the ARF-p53 pathway. Since MDM2 prevents TP53 activity and promotes TP53 degradation by acting as a ubiquitin ligase, the agent that inhibits TP53 can also be a chemical activator of MDM2 or a related ubiquitn ligase that promotes the degradation of TP53, or a chemical activator that enhances MDM2's interaction with TP53.

Contact of a chemical inhibitor of TP53 or a TP53 pathway inhibitor with a cell can be performed by dissolving the inhibitor at an appropriate concentration in an aqueous or non-aqueous solvent, adding the solution of the inhibitor to a medium suitable for cultivation of cells isolated from a human or mouse (for example, minimal essential medium (MEM), Dulbecco's modified Eagle medium (DMEM), RPM11640 medium, 199 medium, F12 medium and the like supplemented with about 5 to 20% fetal bovine serum) so that the inhibitor concentration will fall in a range that fully inhibits the TP53 function and does not cause cytotoxicity, and culturing the cells for a given period. The inhibitor concentration vanes depending on the kind of inhibitor used, and is chosen as appropriate over the range of about 0.1 nM to about 100 nM. Duration of contact is not particularly limited, usually, the inhibitor may be allowed to co-present in the medium until a desired colony emerges.

The TP53 gene is known as a tumor suppressor gene; permanent inhibition of TP53 function potentially increases the risk of carcinogenesis. Chemical inhibitors of TP53 are useful, not only because of the advantage of permitting introduction into cells simply by the addition to the medium, but also because of the ability to terminate the inhibition of TP53 function, easily and quickly, by removing the medium containing the inhibitor.

### Aptamers

The agent that inhibits TP53 can be an aptamer. Aptamers are usually created by selection of a large random sequence pool, but natural aptamers also exist. Inhibition of the target molecule by an aptamer may occur by binding to the target, by catalytically altering the target, by reacting with the target in a way that modifies/alters the target or the functional activity of the target, by covalently attaching to the target as a suicide inhibitor, by facilitating the reaction between the target and another inhibitory molecule. Oligonucleotide aptamers may be comprised of multiple ribonucleotide units, deoxyribonucleotide units, or a mixture of those units. Oligonucleotide aptamers may further comprise one or more modified bases, sugars, phosphate backbone units. Peptide aptamers are small, highly stable proteins that provide a high affinity binding surface for a specific target protein. They usually consist of a protein scaffold with variable peptide loops attached at both ends. The variable loop is typically composed of ten to twenty amino acids, and the scaffold can be any protein that has good solubility and compacity properties. This double structural constraint greatly increases the binding affinity of the peptide aptamer to its target protein. Aptamers can be designed to target TP53 protein.

### Antisense Oligonucleotides

The agent that inhibits TP53 can be an antisense oligonucleotide. Antisense oligonucleotids can be DNA, RNA, a DNA-RNA chimera, or a derivative thereof. Upon hybridizing with complementary bases in an RNA or DNA molecule of interest, antisense oligonucleotids can interfere with the transcription or translation of the target gene, e.g., by inhibiting or enhancing mRNA transcription, mRNA splicing, mRNA transport, or mRNA translation or by decreasing mRNA stability. As presently used, “antisense” broadly includes RNA-RNA interactions, RNA-DNA interactions, and RNaseH mediated arrest. Antisense nucleic acid molecules can be encoded by a recombinant gene for expression in a cell (see, e.g., U.S. Pat. Nos. 5,814,500 and 5,811,234), or alternatively they can be prepared synthetically (see, e.g., U.S. Pat. No. 5,780,607).

### RNAi Agents

The agent that inhibits TP53 can be a RNAi agent. A “RNA agent” can be an siRNA (short inhibitory RNA), shRNA (short or small hairpin RNA), iRNA (interference RNA) agent, RNAi (RNA interference) agent, dsRNA (double-stranded RNA), microRNA, and the like, which specifically binds to a target gene, and which mediates the targeted cleavage of another RNA transcript via an RNA-induced silencing complex (RISC) pathway. In some embodiments, the RNAi agent is an oligonucleotide composition that activates the RISC complex/pathway. In some embodiments, the RNAi agent comprises an antisense strand sequence (antisense oligonucleotide). In some embodiments, the RNAi comprises a single strand. This single-stranded RNAi agent oligonucleotide or polynucleotide can comprise the sense or antisense strand, as described by Sioud 2005 J. Mol. Bio. 348:1079-1090, and references therein. Thus the disclosure encompasses RNAi agents with a single strand comprising either the sense or the antisense strand of an RNAi agent described herein. The use of the RNAi agent to a target gene results in a decrease of target activity, level and/or expression, e.g., a “knock-down” or “knock-out” of the target gene or target sequence.

RNA interference is a post-transcriptional, targeted gene-silencing technique that, usually, uses double-stranded RNA (dsRNA) to degrade messenger RNA (mRNA) containing the same sequence as the dsRNA. The process of RNAi occurs naturally when ribonuclease III (Dicer) cleaves longer dsRNA into shorter fragments called siRNAs. Naturally-occurring siRNAs (small interfering RNAs) are typically about 21 to 23 nucleotides long and comprise about 19 base pair duplexes. The smaller RNA segments then mediate the degradation of the target mRNA. Dicer has also been implicated in the excision of 21- and 22-nucleotide small temporal RNAs (stRNAs) from precursor RNA of conserved structure that are implicated in translational control. Hutvagner et al. 2001, Science, 293, 834. The RNAi response also features an endonuclease complex, commonly referred to as an RNA-induced silencing complex (RISC), which mediates cleavage of single-stranded mRNA complementary to the antisense strand of the siRNA. Cleavage of the target RNA takes place in the middle of the region complementary to the antisense strand of the siRNA duplex.

“RNAi” (RNA interference) has been studied in a variety of systems. Early work in Drosophila embryonic lysates (Elbashir et al. 2001 EMBO J. 20: 6877 and Tuschl et al. International PCT Publication No. WO 01/75164) revealed certain parameters for siRNA length, structure, chemical composition, and sequence that are beneficial to mediate efficient RNAi activity. These studies have shown that 21-nucleotide siRNA duplexes are most active when containing 3′-terminal dinucleotide overhangs. Substitution of the 3′-terminal siRNA overhang nucleotides with 2′-deoxy nucleotides (2′-H) was tolerated. In addition, a 5′-phosphate on the target-complementary strand of a siRNA duplex is usually required for siRNA activity. Later work showed that a 3′-terminal dinucleotide overhang can be replaced by a 3′ end cap, provided that the 3′ end cap still allows the molecule to mediate RNA interference; the 3′ end cap also reduces sensitivity of the molecule to nucleases. See, for example, U.S. Pat. Nos. 8,097,716; 8,084,600; 8,404,831; 8,404,832; and 8,344,128. Additional later work on artificial RNAi agents showed that the strand length could be shortened, or a single-stranded nick could be introduced into the sense strand. In addition, mismatches can be introduced between the sense and anti-sense strands and a variety of modifications can be used. Any of these and various other formats for RNAi agents known in the art can be used to produce RNAi agents to TP53.

In some embodiments, the RNAi agent is ligated to one or more diagnostic compound, reporter group, cross-linking agent, nuclease-resistance conferring moiety, natural or unusual nucleobase, lipophilic molecule, cholesterol, lipid, lectin, steroid, uvaol, hecigenin, diosgenin, terpene, triterpene, sarsasapogenin, Friedelin, epifriedelanol-derivatized lithocholic acid, vitamin, carbohydrate, dextran, pullulan, chitin, chitosan, synthetic carbohydrate, oligo lactate 15-mer, natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

Kits for RNAi synthesis are commercially available, e.g., from New England Biolabs and Ambion.

A suitable RNAi agent can be selected by any process known in the art or conceivable by one of ordinary skill in the art. For example, the selection criteria can include one or more of the following steps: initial analysis of the gene sequence and design of RNAi agents; this design can take into consideration sequence similarity across species (human, cynomolgus, mouse, etc.) and dissimilarity to other genes; screening of RNAi agents in vitro (e.g., at 10 nM in cells); determination of EC50 in HeLa cells; determination of viability of various cells treated with RNAi agents, wherein it is desired that the RNAi agent to a target molecule does not inhibit the viability of these cells; testing with human PBMC (peripheral blood mononuclear cells), e.g., to test levels of TNF-alpha to estimate immunogenicity, wherein immunostimulatory sequences are less desired; testing in human whole blood assay, wherein fresh human blood is treated with an RNAi agent and cytokine/chemokine levels are determined [e.g., TNF-alpha (tumor necrosis factor-alpha) and/or MCP1 (monocyte chemotactic protein 1)], wherein immunostimulatory sequences are less desired; determination of gene knock down in vive using subcutaneous tumors in test animals; target gene modulation analysis, e.g., using a pharmacodynamic (PD) marker, and optimization of specific modifications of the RNAi agents.

RNAi agents can be delivered or introduced (e.g., to a cell in vitro or to a patient) by any means known in the art. “Introducing into a cell,” when referring to an iRNA, means facilitating or effecting uptake or absorption into the cell, as is understood by those skilled in the art. Absorption or uptake of an iRNA can occur through unaided diffusive or active cellular processes, or by auxiliary agents or devices. The meaning of this term is not limited to cells in vitro; an iRNA may also be “introduced into a cell,” wherein the cell is part of a living organism. In such an instance, introduction into the cell will include the delivery to the organism. For example, for in vive delivery, iRNA can be injected into a tissue site or administered systemically. In vive delivery can also be achieved by a beta-glucan delivery system, such as those described in U.S. Pat. Nos. 5,032,401 and 5,607,677, and U.S. Publication No. 2005/0281781 which are hereby incorporated by reference in their entirety. In vitro introduction into a cell includes methods known in the art such as electroporation and lipofection. Further approaches are described below or known in the art.

Delivery of RNAi agent to tissue can be a problem because the material must reach the target organ and must also enter the cytoplasm of target cells. RNA cannot penetrate cellular membranes, so systemic delivery of naked RNAi agent is unlikely to be successful. RNA is quickly degraded by RNAse activity in serum. For these reasons, other mechanisms to deliver RNAi agent to target cells has been devised. Methods known in the art include but are not limited to: viral delivery (retrovirus, adenovirus, lentivirus, baculovirus, AAV); liposomes (Lipofectamine, cationic DOTAP, neutral DOPC) or nanoparticles (cationic polymer, PE1), bacterial delivery (tkRNAi), and also chemical modification (LNA) of siRNA to improve stability. Xia et al. 2002 Nat. Biotechnol. 20 and Devroe et al. 2002. BMC Biotechnol. 21: 15, disclose incorporation of siRNA into a viral vector. Other systems for delivery of RNAi agents are contemplated, and the RNAi agents of the present invention can be delivered by various methods yet to be found and/or approved by the FDA or other regulatory authorities.

Liposomes have been used previously for drug delivery (e.g., delivery of a chemotherapeutic). Liposomes (e.g., cationic liposomes) are described in PCT publications WO02/100435A1, WO03/015757A1, and WO04029213A2; U.S. Pat. Nos. 5,962,016; 5,030,453; and 6,680,068; and U.S. Patent Application 2004/0208921. A process of making liposomes is also described in WO04/002453A1. Furthermore, neutral lipids have been incorporated into cationic liposomes (e.g., Farhood et al. 1995). Cationic liposomes have been used to deliver RNAi agent to various cell types (Sioud and Sorensen 2003; U.S. Patent Application 2004/0204377; Duxbury et al., 2004; Donze and Picard, 2002). Use of neutral liposomes disclosed in Miller et al. 1998, and U.S. Publ. 2003/0012812.

As used herein, the term “SNALP” refers to a stable nucleic acid-lipid particle. A SNALP represents a vesicle of lipids coating a reduced aqueous interior comprising a nucleic acid such as an iRNA or a plasmid from which an iRNA is transcribed. SNALPs are described, e.g., in U.S. Patent Application Publication Nos. 20060240093, 20070135372, and in International Application No. WO 2009082817. These applications are incorporated herein by reference in their entirety.

Chemical transfection using lipid-based, amine-based and polymer-based techniques, is disclosed in products from Ambion Inc., Austin, Tex.; and Novagen, EMD Biosciences, Inc., an Affiliate of Merck KGaA, Darmstadt, Germany); Ovcharenko D (2003) “Efficient delivery of siRNAs to human primary cells.” Ambion TechNotes 10 (5): 15-16). Additionally, Song et al. (Nat Med. published online (Fete I 0, 2003) doi: 10.1038/nm828) and others [Caplen et al. 2001 Proc. Natl. Acad. Sci. (USA), 98: 9742-9747; and McCaffrey et al. Nature 414: 34-39] disclose that liver cells can be efficiently transfected by injection of the siRNA into a mammars circulatory system.

A variety of molecules have been used for cell-specific RNAi agent delivery. For example, the nucleic acid-condensing property of protamine has been combined with specific antibodies to deliver siRNAs. Song et al. 2005 Nat Biotch. 23: 709-717. The self-assembly PEGylated polycation polyethylenimine has also been used to condense and protect siRNAs. Schiffelers et al., 2004 Nucl. Acids Res. 32: 49, 141-110.

The siRNA-containing nanoparticles were then successfully delivered to integrin overexpressing tumor neovasculature. Hu-Lieskovan et al., 2005 Cancer Res. 65: 8984-8992.

The RNAi agents of the present invention can be delivered via, for example, Lipid nanoparticles (LNP); neutral liposomes (NL); polymer nanoparticles; double-stranded RNA binding motifs (dsRBMs); or via modification of the RNAi agent (e.g., covalent attachment to the dsRNA).

Lipid nanoparticles (LNP) are self-assembling cationic lipid based systems. These can comprise, for example, a neutral lipid (the liposome base); a cationic lipid (for siRNA loading); cholesterol (for stabilizing the liposomes); and PEG-lipid (for stabilizing the formulation, charge shielding and extended circulation in the bloodstream). The cationic lipid can comprise, for example, a headgroup, a linker, a tail and a cholesterol tail. The LNP can have, for example, good tumor delivery, extended circulation in the blood, small particles (e.g., less than 100 nm), and stability in the tumor microenvironment (which has low pH and is hypoxic). Neutral liposomes (NL) are non-cationic lipid based particles. Polymer nanoparticles are self-assembling polymer-based particles. Double-stranded RNA binding motifs (dsRBMs) are self-assembling RNA binding proteins, which will need modifications.

### Ribozymes

The agent that inhibits TP53 can be a ribozyme. Ribozymes are catalytic RNA molecules capable of cleaving RNA substrates. Ribozyme specificity is dependent on complementary RNA-RNA interactions (for a review, see Cech and Bass, Annu. Rev. Biochem. 1986; 55: 599-629). Two types of ribozymes, hammerhead and hairpin, have been described. Each has a structurally distinct catalytic center. Ribozyme technology is described further in Intracellular Ribozyme Applications: Principals and Protocols, Rossi and Couture ed., Horizon Scientific Press, 1999.

### Decoy Molecule

The agent that inhibits TP53 can be a decoy molecule, e.g., a decoy nucleic acid comprising a consensus sequence of TP53-responsive element (e.g., Pu-Pu-Pu-G-AT-T/A-C-Py-Py-Py (Pu: purine base, Py: pyrimidine base); SEQ ID NO: 10). Such a nucleic acid can be synthesized using an automated DNA/RNA synthesizer. Alternatively, such a decoy nucleic acid is commercially available (e.g., p53 transcription factor decoy from GeneDetect.com).

### Antibody

The agent that inhibits TP53 can be an antibody or derivative thereof, or a nucleic acid encoding an antibody or derivative that specifically binds TP53. For example, the agent that inhibits TP53 can be an anti-p53 neutralizing or antagonist antibody or nucleic acid that encodes such an antibody. The antibody can be a polyclonal or monoclonal antibody.

A naturally occurring antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VI regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2) or subclass.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively. In particular, the term “antibody” specifically includes an IgG-scFv format.

The term “epitope binding domain” or “EBD” refers to portions of a binding molecule (e.g., an antibody or epitope-binding fragment or derivative thereof), that specifically interacts with (e.g., by binding, steric hindrance, stabilizing/destabilizing, spatial distribution) a binding site on a target epitope. EBD also refers to one or more fragments of an antibody that retain the ability to specifically interact with (e.g., by binding, steric hindrance, stabilizing/destabilizing, spatial distribution) a TP53 epitope and inhibit signal transduction. Examples of antibody fragments include, but are not limited to, an scFv, a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward at al., (1989) Nature 341:544-546), which consists of a VH domain; and an isolated complementarity determining region (CDR).

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird at al., (1988) Science 242:423-426; and Huston et al., (1988) Proc. Natl. Acad. Sci. 85:5879-5883).

Such single chain antibodies are also intended to be encompassed within the terms “fragment”, “epitope-binding fragment” or “antibody fragment.” These fragments are obtained using conventional techniques known to those of skill in the art, and the fragments are screened for utility in the same manner as are intact antibodies.

Antibody fragments can be incorporated into single chain molecules comprising a pair of tandem Fv segments (VH-CH1-VH-CH1) which, together with complementary light chain polypeptides, form a pair of antigen binding regions (Zapata et al., (1995) Protein Eng. 8:1057-1062; and U.S. Pat. No. 5,641,870), and also include Fab fragments, F(ab) fragments, and anti-idiotypic (anti-Id) antibodies (including, e.g., anti-Id antibodies to antibodies of the invention), and epitope-binding fragments of any of the above.

EBDs also include single domain antibodies, maxibodies, unibodies, minibodies, triabodies, tetrabodies, v-NAR and bis-scFv, as is known in the art (see, e.g., Hollinger and Hudson, (2005) Nature Biotechnology 23: 1126-1136), bispecific single chain diabodies, or single chain diabodies designed to bind two distinct epitopes. EBDs also include antibody-like molecules or antibody mimetics, which include, but not limited to minibodles, maxybodles, Fn3 based protein scaffolds, Ankrin repeats (also known as DARpins), VASP polypeptides, Avian pancreatic polypeptide (aPP), Tetranectin, Affililin, Knottins, SH3 domains, PDZ domains, Tendamistat, Neocarzinostatin, Protein A domains, Lipocalins, Transferrin, and Kunitz domains that specifically bind epitopes, which are within the scope of the invention. Antibody fragments can be grafted into scaffolds based on polypeptides such as Fibronectin type III (Fn3) (see U.S. Pat. No. 6,703,199, which describes fibronectin polypeptide monobodies).

An isolated antibody can be a monovalent antibody, bivalent antibody, multivalent antibody, bivalent antibody, biparatopic antibody, bispecific antibody, monoclonal antibody, human antibody, recombinant human antibody, or any other type of antibody or epitope-binding fragment or derivative thereof.

The phrase “isolated antibody,” as used herein, refers to antibody that is substantially free of other antibodies having different antigenic specificities (e.g., an isolated antibody that specifically binds TP53 is substantially free of antibodies that specifically bind antigens other than TP53). An isolated antibody that specifically binds a target molecule may, however, have cross-reactivity to the same antigens from other species, e.g., an isolated antibody that specifically binds TP53 may bind TP53 molecules from other species. Moreover, an isolated antibody may be substantially free of other cellular material and/or chemicals.

The term “monovalent antibody” as used herein, refers to an antibody that binds to a single epitope on a target molecule.

The term “bivalent antibody” as used herein, refers to an antibody that binds to two epitopes on at least two identical target molecules. The bivalent antibody may also crosslink the target molecules to one another. A “bivalent antibody” also refers to an antibody that binds to two different epitopes on at least two identical target molecules.

The term “multivalent antibody” refers to a single binding molecule with more than one valency, where “valency” is described as the number of antigen-binding moieties present per molecule of an antibody construct. As such, the single binding molecule can bind to more than one binding site on a target molecule. Examples of multivalent antibodies include, but are not limited to bivalent antibodies, trivalent antibodies, tetravalent antibodies, pentavalent antibodies, and the like, as well as bispecific antibodies and biparatopic antibodies. For example, for TP53, the multivalent antibody (e.g., a TP53 biparatopic antibody) has a binding moiety for two domains of TP53, respectively.

The term “multivalent antibody” also refers to a single binding molecule that has more than one antigen-binding moiety for two separate target molecules. For example, an antibody that binds to TP53 and a second target molecule that is not TP53. In one embodiment, a multivalent antibody is a tetravalent antibody that has four epitope binding domains. A tetravalent molecule may be bispecific and bivalent for each binding site on that target molecule.

The term “biparatopic antibody” as used herein, refers to an antibody that binds to two different epitopes on a single target molecule. The term also includes an antibody, which binds to two domains of at least two target molecules, e.g., a tetravalent biparatopic antibody.

The term “bispecific antibody” as used herein, refers to an antibody that binds to two or more different epitopes on at least two different targets (e.g., TP53 and a target that is not TP53).

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

The phrase “human antibody,” as used herein, includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Al Lazikani et al., (1997) J. Mol. Bio. 273:927 948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mal. Biol. 273:927-948.

The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

The phrase “recombinant human antibody” as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VI. regions of the recombinant antibodies are sequences that, while derived from and related to human germline VH and VL sequences, may not naturally exist within the human antibody germline repertoire in vivo.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region, may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a Ckappa/lambda region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

The term “binding site” as used herein comprises an area on a target molecule to which an antibody or antigen binding fragment selectively binds.

The term “epitope” as used herein refers to any determinant capable of binding with high affinity to an immunoglobulin. An epitope is a region of an antigen that is bound by an antibody that specifically targets that antigen, and when the antigen is a protein, includes specific amino acids that directly contact the antibody. Most often, epitopes reside on proteins, but in some instances, may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics.

Generally, antibodies specific for a particular target antigen will bind to an epitope on the target antigen in a complex mixture of proteins and/or macromolecules.

As used herein, the term “affinity” refers to the strength of interaction between antibody and antigen at single antigenic sites. Within each antigenic site, the variable region of the antibody “arm” interacts through weak non-covalent forces with the antigen at numerous sites; the more interactions, the stronger the affinity. As used herein, the term “high affinity” for an IgG antibody or fragment thereof (e.g., a Fab fragment) refers to an antibody having a knock down of 10⁻⁸ M or less, 10⁻⁹ M or less, or 10⁻¹⁰ M, or 10⁻¹¹ M or less, or 10⁻¹² M or less, or 10⁻¹³ M or less for a target antigen. However, high affinity binding can vary for other antibody isotypes. For example, high affinity binding for an IgM isotype refers to an antibody having a knock down of 10⁻⁷ M or less, or 10⁻⁸ M or less.

As used herein, the term “avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. Similarly, conformational epitopes are readily identified by determining spatial conformation of amino acids such as by, e.g., x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. Mol. Biol. 157:105-132; for hydropathy plots.

### Nucleic Acids and Vectors

Nucleic acid sequences encoding a gene editing system and/or TP53 inhibitor as described herein can be obtained using standard synthetic and/or recombinant techniques. Desired nucleic acid sequences may be isolated and sequenced from appropriate source cells or can be synthesized using nucleotide synthesizer or PCR techniques.

The expression of natural or synthetic nucleic acids encoding a gene editing system and/or TP53 inhibitor as described herein is typically achieved by operably linking a nucleic acid encoding the gene editing system and/or TP53 inhibitor as described herein to a promoter, and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. In one aspect, the vector comprising a gene editing system and/or TP53 inhibitor as described herein is a DNA, a RNA, a plasmid, an adenoviral vector, a lentivirus vector, or a retrovirus vector.

Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. Selection of an appropriate vector will depend mainly on the size of the nucleic acids to be inserted into the vector and the particular host cell to be transformed with the vector. Each vector contains various components, depending on its function (amplification or expression of heterologous nucleic acid sequence, or both) and its compatibility with the particular host cell in which it resides. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193). Other elements that may be included in the vector include a ribosomal binding site, a signal sequence, a transcriptional termination site, a tag, and a reporter gene.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to desired host cells, or cells of the subject, either in vivo or ex vivo. A number of retroviral systems are known in the art. In some aspects, adenovirus vectors are used. A number of adenovirus vectors are known in the art. In one aspect, adeno-associated virus (AAV) vector, e.g., an AAV2, AAV3, AAV4, AAV5, AAV6, AAV7, AAV8, or AAV9 vector, or any modified vectors thereof. In one aspect, lentivirus vectors are used.

### Expression in Cells

The present invention provides gene editing systems in combination with TP53 inhibitors are useful in engineering cells to express such systems and TP53 inhibitors, and in applications involving the use of such engineered cells. The cells may be eurkaryote cells, e.g., insect, worm or mammalian cells. Suitable mammalian cells include, but are not limited to, equine, bovine, ovine, canine, feline, murine, non-human primate cells, and human cells.

Among the various species, various types of cells may be used, such as hematopoietic, neural, glial, mesenchymal, cutaneous, mucosal, stromal, muscle (including smooth muscle cells), spleen, reticulo-endothelial, epithelial, endothelial, hepatic, kidney, gastrointestinal, pulmonary, fibroblast, and other cell types. Other cells for use in the present invention include stem and progenitor cells, such as hematopoietic, neural, stromal, muscle, hepatic, pulmonary, gastrointestinal and mesenchymal stem or progenitor cells. In some aspects using hematopoietic cells, the hematopoietic cells may include any of the nucleated cells which may be involved with the erythroid, lymphoid or myelomonocytic lineages, as well as myoblasts and fibroblasts, and immune effector cells, e.g., T cells and NK cells. The cells may be autologous cells, syngeneic cells, allogeneic cells and even in some cases, xenogeneic cells with respect to an intended host organism.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a nucleic acid into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A preferred method for the introduction of a polynucleotide into a host cell is lipofection, e.g., using Lipofectamine (Life Technologies).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

Transfer of a gene editing system and/or a TP53 inhibitor into a cell can be achieved using a method suitable for delivering a particular type of molecules to the cell. For example, they can be transferred into a cell by microinjection, by using a protein transfer reagent, or by fusing to a protein transfer domain (PTD) or cell penetrating peptide (CPP). Protein transfer reagents are commercially available, including those based on a cationic lipid, such as BioPOTER Protein Delivery Reagent (Gene Therapy Systems), Pro-Ject™.

Protein Transfection Reagent (PIERCE) and ProVectin (IMGENEX); those based on a lipid, such as Profect-1 (Targeting Systems); those based on a membrane-permeable peptide, such as Penetrain Peptide (Q biogene) and Chariot Kit (Active Motif), GenomONE (ISHIHARA SANGYO KAISHA, LTD) utilizing HVJ envelope (inactivated hemagglutinating virus of Japan) and the like. The transfer can be achieved by following the protocols attached to these reagents.

Developed PTDs include those using transcellular domains of proteins such as drosophila-derived AntP, HIV-derived TAT (Frankel, A. et al, Cell 55, 1189-93 (1988) or Green, M. & Loewenstein, P. M. Cell 55, 1179-88 (1988)), Penetratin (Derossi, D. et al, J. Biol. Chem. 269, 10444-50 (1994)), Buforin II (Park, C. B. et al. Proc. Natl Acad. Sci. USA 97, 8245-50 (2000)), Transportan (Pooga, M. et al. FASEB J. 12, 67-77 (1998)), MAP (model amphipathic peptide) (Oehlke, J. et al. Biochim. Biophys. Acta. 1414, 127-39 (1998)), K-FGF (Lin, Y. Z. et al. J. Biol. Chem. 270, 14255-14258 (1995)), Ku70 (Sawada, M. et al. Nature Cell Biol. 5, 352-7 (2003)), Prion (Lundberg, P. et al. Biochem. Biophys. Res. Commun. 299, 85-90 (2002)), pVEC (Elmquist, A. et al. Exp. Cell Res. 269, 237-44 (2001)), Pep-1 (Morris, M. C. et al. Nature Biotechnol. 19, 1173-6 (2001)), Pep-7 (Gao, C. et al. Bioorg. Med. Chem. 10, 4057-65 (2002)), SynBI (Rousselle, C. et al. Mol. Pharmacol. 57, 679-86 (2000)), HN-I (Hong, F. D. & Clayman, G L. Cancer Res. 60, 6551-6 (2000)), and HSV-derived VP22. CPPB derived from the PTDs include polyarginines such as 11R (Cell Stem Cell, 4, 381-384 (2009)) and 9R (Cell Stem Cell, 4, 472-476 (2009)).

A fusion protein expression vector incorporating cDNA of the molecules of the invention (e.g., a gene editing system and/or a TP53 inhibitor) and PTD or CPP sequence can be prepared and used to recombinantly express the fusion protein. The fused protein is recovered and used for transfer. Transfer can be performed in the same manner as above except that a protein transfer reagent is not added.

The protein transferring operation can be performed one or more optionally chosen times (e.g., once or more to 10 times or less, or once or more to 5 times or less and the like). Preferably, the transferring operation can be performed twice or more (e.g., 3 times or 4 times) repeatedly. The time interval for repeated transferring operation is, for example, 6 to 48 hours, preferably 12 to 24 hours. According to the present invention, a TP53 inhibitor of the invention can be introduced into a cell prior to, at the same time, or after the introduction of a gene editing system used in accordance of the present invention.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (‘DMPC’) can be obtained from Sigma, St. Louis, Mo.; dicetyl phosphate (‘DCP’) can be obtained from K & K Laboratories (Plainview, N.Y.); cholesterol (“Choi”) can be obtained from Calbiochem-Behring; dimyristyl phosphatidyiglycerol (‘DMPG’) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In some aspects, host cells can be modified ex vivo with a nucleic acid, e.g., vector, comprising the molecules described herein. Cells which have been modified ex vivo with the vector may be grown in culture under selective conditions and cells which are selected as having the desired construct(s) may then be expanded and further analyzed, using, for example, the polymerase chain reaction for determining the presence of the construct in the host cells and/or assays for the production of the desired gene product(s). Once modified host cells have been identified, they may then be used as planned, e.g. grown in culture or introduced into a host organism.

Depending upon the nature of the cells, the cells may be introduced into a host organism, e.g. a mammal, e.g., a human, in a wide variety of ways. Hematopoietic cells may be administered by injection into the vascular system, there being usually at least about 104 cells and generally not more than about 1010 cells. The number of cells which are employed will depend upon a number of circumstances, the purpose for the introduction, the lifetime of the cells, the protocol to be used, for example, the number of administrations, the ability of the cells to multiply, the stability of the therapeutic agent, the physiologic need for the therapeutic agent, and the like. Generally, for myoblasts or fibroblasts for example, the number of cells will be at least about 104 and not more than about 109 and may be applied as a dispersion, generally being injected at or near the site of interest. The cells will usually be in a physiologically-acceptable medium. Cells engineered in accordance with this invention may also be encapsulated, e.g. using conventional biocompatible materials and methods, prior to implantation into the host organism or patient for the production of a therapeutic protein.

In other aspects, the cells can be engineered to express the gene editing system and/or TP53 inhibitor as described herein in vivo. For this purpose, various techniques have been developed for modification of target tissue and cells in vivo. A number of viral vectors have been developed, such as adenovirus, adeno-associated virus, and retroviruses, as discussed above, which allow for transfection and, in some cases, integration of the virus into the host. See, for example, Dubensky et al. (1984) Proc. Natl. Acad. Sci. USA 81, 7529-7533; Kaneda et al., (1989) Science 243, 375-378; Hiebert et al. (1989) Proc. Natl. Acad. Sci. USA 86, 3594-3598; Hatzoglu et al. (1990) J. Biol. Chem. 265, 17285-17293 and Ferry, et al. (1991) Proc. Natl. Acad. Sci. USA 88, 8377-8381. The vector may be administered by injection, e.g. intravascularly or intramuscularly, inhalation, or other parenteral mode. Non-viral delivery methods such as administration of the DNA via complexes with liposomes or by injection, catheter or biolistics may also be used.

In accordance with in vivo genetic modification, the manner of the modification will depend on the nature of the tissue, the efficiency of cellular modification required, the number of opportunities to modify the particular cells, the accessibility of the tissue to the nucleic acid, e.g., vector, composition to be introduced, and the like. Nucleic acid introduction need not result in integration. In some situations, transient maintenance of the introduced nucleic acids described herein may be sufficient. In this way, one could have a short term effect, where cells could be introduced into the host and then turned on after a predetermined time, for example, after the cells have been able to home to a particular site.

### Pharmaceutical Compositions and Treatments

Also provided herein are compositions, e.g., pharmaceutical compositions, comprising one or more agents that inhibit TP53 described herein and a gene editing system.

In some embodiments, provided herein are compositions comprising an agent that inhibits TP53 and one or more components of the CRISPR-Cas9 gene editing system. For example, such composition can comprising a TP53 inhibitor, a Cas9 molecule, and a guide RNA (gRNA), e.g., a guide RNA capable of targeting the Cas9 molecule to a target nucleic acid.

Pharmaceutical compositions may comprise one or more agents that inhibit TP53 described herein and a gene editing system, e.g., a polypeptide or a nucleic acid encoding one or more agents that inhibit TP53 described herein and a gene editing system, e.g., a vector encoding one or more agents that inhibit TP53 described herein and a gene editing system, or a cell comprising one or more agents that inhibit TP53 described herein and a gene editing system, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In an aspect, the pharmaceutical compositions are formulated for intravenous administration.

Pharmaceutical compositions may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount,” “an anti-cancer effective amount,” “a cancer-inhibiting effective amount,” or “therapeutic amount” is indicated, the precise amount of the compositions to be administered can be determined by a physician with consideration of individual differences in age, weight, disease state, e.g., tumor size, extent of infection or metastasis, and condition of the patient (subject). Compositions may also be administered multiple times at these dosages. The optimal dosage and treatment regime for a particular patient can be determined by one skilled in the art of medicine by monitoring the patient for signs of disease and adjusting the treatment accordingly.

In another aspect, provided herein are methods of modifying a donor cell or organ for transplantation. Such methods comprise contacting said donor cell or organ with an apoptosis inhibitor, e.g., TP53 inhibitor, and performing gene editing to said donor cell or organ. In some embodiments, such methods further comprise contacting the cell with growth factor, e.g., basic fibroblast growth factor (bFGF). In some embodiments, the donor is a non-human subject, e.g., pig, cow, horse, cat, dog, sheep, or goat.

The present invention provides compositions and methods for the treatment of a variety of diseases and disorders. In some aspects, the disease or disorder is a disease or disorder that is associated with abberant gene expression. In some aspects, the disease or disorder is a genetic disorder. In some aspects, the disease or disorder is a lysosomal storage disorder.

In other aspects, the present invention provides compositions and methods for the treatment of a subject in need thereof of heart, lung, combined heart lung, liver, kidney, pancreatic, skin or comeal transplants, including, but not limited to, allograft rejection or xenograft rejection, and for the prevention of graft versus host disease, such as following bone marrow transplant, and organ transplant associated arteriosclerosis.

The invention also provides compositions and methods for the treatment, prevention, or amelioration of autoimmune disease and of inflammatory conditions, in particular inflammatory conditions with an aetiology including an autoimmune component such as arthritis (for example rheumatoid arthritis, arthritis chronica progrediente and arthritis deformans) and rheumatic diseases, including inflammatory conditions and rheumatic diseases involving bone loss, inflammatory pain, spondyloarhropathies including ankylosing spondylitis, Reiter syndrome, reactive arthritis, psoriatic arthritis, juvenile idiopathic arthritis and enterophathis arthritis, enthesitis, hypersensitivity (including both airways hypersensitivity and dermal hypersensitivity) and allergies. Specific auto immune diseases for which antibodies of the disclosure may be employed include autoimmune haematological disorders (including e.g. hemolytic anaemia, aplastic anaemia, pure red cell anaemia and idiopathic thrombocytopenia), systemic lupus erythematosus (SLE), lupus nephritis, inflammatory muscle diseases (dermatomyosytis), periodontitis, polychondritis, scleroderma, Wegener granulomatosis, dermatomyositis, chronic active hepatitis, myasthenia gravis, psoriasis, Steven Johnson syndrome, idiopathic sprue, autoimmune inflammatory bowel disease (including e.g. ulcerative colitis, Crohn's disease and irritable bowel syndrome), endocrine ophthalmopathy, Graves' disease, sarcoidosis, multiple sclerosis, systemic sclerosis, fibrotic diseases, primary biliary cirrhosis, juvenile diabetes (diabetes mellitus type I), uveitis, keratoconjunctivitis sicca and vernal keratoconjunctivitis, interstitial lung fibrosis, periprosthetic osteolysis, glomerulonephritis (with and without nephrotic syndrome, e.g. including idiopathic nephrotic syndrome or minimal change nephropathy), multiple myeloma other types of tumors, inflammatory disease of skin and cornea, myositis, loosening of bone implants, metabolic disorders, (such as obesity, atherosclerosis and other cardiovascular diseases including dilated cardiomyopathy, myocarditis, diabetes mellitus type II, and dyslipidemia), and autoimmune thyroid diseases (including Hashimoto thyroiditis), small and medium vessel primary vasculitis, large vessel vasculitides including giant cell arteritis, hidradenitis suppurativa, neuromyelitis optica, Sjögren's syndrome, Behcet's disease, atopic and contact dermatitis, bronchiolitis, inflammatory muscle diseases, autoimmune peripheral neurophaties, immunological renal, hepatic and thyroid diseases, inflammation and atherothrombosis, autoinflammatory fever syndromes, immunohematological disorders, and bullous diseases of the skin and mucous membranes. Anatomically, uveitis can be anterior, intermediate, posterior, or pan-uveitis. It can be chronic or acute. The etiology of uveitis can be autoimmune or non-infectious, infectious, associated with systemic disease, or a white-dot syndrome.

The present invention also provides compositions and methods for the treatment, prevention, or amelioration of asthma, bronchitis, bronchiolitis, idiopathic interstitial pneumonias, pneumoconiosis, pulmonary emphysema, and other obstructive or inflammatory diseases of the airways.

The present invention also provides compositions and methods for treating diseases of bone metabolism including osteoarthritis, osteoporosis and other inflammatory arthritis, and bone loss in general, including age-related bone loss, and in particular periodontal disease.

In addition, the present invention provides compositions and methods for treating chronic candidiasis and other chronic fungal diseases, as well as complications of infections with parasites, and complications of smoking are considered to be promising avenues of treatment, as well as viral infection and complications of viral infection (e.g., HIV infection).

The present invention also provides compositions and methods for treating breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors schwannoma, head and neck cancer, bladder cancer, esophageal cancer, Barretts esophageal cancer, glioblastoma, clear cell sarcoma of soft tissue, malignant mesothelioma, neurofibromatosis, renal cancer, melanoma, prostate cancer, benign prostatic hyperplasia (BPH), gynacomastica, and endometriosis.

In some aspects, the gene editing system comprising a TP53 inhibitor as described herein can be used to create an allogeneic immune cell, e.g., a T-cell or NK cell, e.g., an allogeneic immunce cell lacking expression of a functional T cell receptor (TCR) and/or human leukocyte antigen (HLA), e.g., HLA class I and/or HLA class II. The cells edited by the gene editing system according to the present invention may further comprise a chimeric antigen receptor (“CAR”).

In some aspects, the gene editing system comprising a TP53 inhibitor as described herein can be used to regulate, e.g., downregulate, inhibit or repress expression of an inhibitory molecule. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4 and TGFR beta. Inhibition of an inhibitory molecule in a cell, e.g., with the use of a gene editing system as described herein, can improve the function of the cell.

In some aspects, the gene editing system comprising a TP53 inhibitor as described herein can be used to treat a disorder associated with abberant gene expression, e.g., a cancer or a genetic disorder. Examples of cancers that may be treated with the compositions of the present invention include breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors schwannoma, head and neck cancer, bladder cancer, esophageal cancer, Barretts esophageal cancer, glioblastoma, clear cell sarcoma of soft tissue, malignant mesothelioma, neurofibromatosis, renal cancer, melanoma, prostate cancer, benign prostatic hyperplasia (BPH), gynacomastica, and endometriosis. Examples of genetic disorders are described on the website of the National Institutes of Health under the topic subsection Genetic Disorders (website at health.nih.gov/topic/GeneticDisorders). Other examples include ocular defects caused by genetic mutations, including those described in Genetic Diseases of the Eye, Second Edition, edited by Elias I. Traboulsi, Oxford University Press, 2012. Preferably the genetic disorder is selected from the group consisting of epidermolysis bullosa, recessive dystrophic epidermolysis bullosa (RDEB), osteogenesis imperfecta, dyskeratosis congenital, a mucopolysaccharidosis, muscular dystrophy, cystic fibrosis (CFTR), fanconi anemia, a sphingolipidosis, a lipofuscinosis, adrenoleukodystrophy, severe combined immunodeficiency, sickle-cell anemia and thalassemia.

In some aspects, the gene editing system comprising a TP53 inhibitor as described herein can be used to treat a lysosomal storage disorder. Examples of liposomal storage disorders include Activator Deficiency/GM2 Gangliosidosis, Alpha-mannosidosis, Aspartylglucosaminuria, Cholesteryl ester storage disease, Chronic Hexosaminidase A Deficiency, Cystinosis, Danon disease, Fabry disease, Farber disease, Fucosidosis, Galactosialidosis, Gaucher Disease, GM1 gangliosidosis, I-Cell disease/Mucolipidosis II, Infantile Free Sialic Acid Storage Disease/ISSD, Juvenile Hexosaminidase A Deficiency, Krabbe disease, Metachromatic Leukodystrophy, Mucopolysaccharidoses disorders, e.g., Pseudo-Hurler polydystrophy/Mucolipidosis IIIA, MPSI Hurler Syndrome, MPSI Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type A/MPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC, Mucolipidosis type IV; Multiple sulfatase deficiency, Niemann-Pick Disease, Neuronal Ceroid Lipofuscinoses, CLN6 disease, Batten-Spielmeyer-Vogt/Juvenile NCL/CLN3 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis, Pompe disease/Glycogen storage disease type II, Pycnodysostosis, Sandhoff disease/Adult Onset/GM2 Gangliosidosis, Schindler disease, Salla disease/Sialic Acid Storage Disease, Tay-Sachs/GM2 gangliosidosis, and Wolman disease.

In some aspects, the gene editing system comprising a TP53 inhibitor as described herein can be used to decrease the toxicity of the gene editing component and/or to increase gene editing efficiency when the gene editing component is used to modify the nucleic acid of a target gene and/or to modulating the expression of a target gene. In one aspect, the gene editing system comprising a TP53 inhibitor as described herein can be used to decrease the toxicity of the gene editing component and/or to increase gene editing efficiency when the gene editing component is used to modifying cells, tissues and organs for transplantation to a subject in need thereof. Such cells, tissues and organs can be for allotransplantation or for xenotransplantation. In one aspect, the gene editing system comprising a TP53 inhibitor as described herein can be used to decrease the toxicity of the gene editing component when it is used to reduce immunological incompatibitliy between the donor organ and the transplant recipient and/or to reduce rejection due to viral infection of the recipient by the donor organ. In a specific embodiment, the donor organ can be from a pig and be used in a xenotransplant to a human recipient, wherein the gene editing system can be used to silence porcine genes involved in hyperacute rejection, delayed xenograft rejection, cellular rejection and/or chronic rejection. For example, genes involved in hyperacute rejection, which have been successfully knocked out by a gene editing system as described herein, include α1,3-galactosyltransferase (GGTA1), cytidine monophosphate-N-acetylneuraminic acid hydroxylase (CMAH) and β1,4-N-acetyl-galactosaminyltransferase (β4GalNT2) (Petersen et al. (2016) Xenotransplantation 23(5): 338-46; Estrada et al. (2015) Xenotransplantation 22(3): 194-202). In another specific embodiment, the donor organ can be from a pig and be used in a xenotransplant to a human recipient, wherein the gene editing system is used to reduce or eliminate viral transmission between the pig organ and the transplant recipient. For example, to reduce the risk of cross-species transmission of porcine endogenous retroviruses (PERVs), the gene editing system can be used to inactivate PERVs and therefore eliminate transmission of some or all PERVs to the human recipient. Inclusion of a TP53 inhibitor in combination with the gene editing system can reduce the stress from multiplex DNA damage during genome editing to inactivate PERVs and support the expansion of PERV-inactivated cells.

### Kits

Also provided herein are kits including one or more of the compositions provided herein and instructions for use. Kits as provided herein can be used in accordance with any of the methods described above. Those skilled in the art will be aware of other suitable uses for kits provided herein, and will be able to employ the kits for such uses. Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

One skilled in the art will recognize many methods and materials similar or equivalent to those described herein, which could be used in the practice of the present invention. Indeed, the present invention is in no way limited to the methods and materials described.

### EXAMPLES

The invention is further described in the following examples, which do not limit the scope of the invention described in the claims.

### Example 1: Materials and Methods

hPSC Culture and Inducible Cas9 Cell Line Generation

Cells were grown in TeSR-E8 media (STEMCELL TECH.-05940) on tissue-culture plates coated with vitronectin (Gibco-A14700). Passaging was preformed using ReLeSR (STEMCELL TECH.-05873) or accutase (Gibco-A1110501) in media with thiazovivin (Selleckchem-S1459, 0.2 uM to 0.8 uM). Cell lines generated, screened and QC'd as described by Wells et al., 2016 (under review). iCas9 and ddCas9 cell lines were maintained in media containing 200 ug/ml G418 (Millipore-345812).

Lentiviral and Lipid Delivery of sgRNAs for Cas9 Mutagenesis

During replating lentiCRISPRs were added to a single cell suspension of 2*10⁵ cells in E8 with thiazovivin without polybrene. After 24 hours cells were maintained in 2 ug/ml to puromycin. At the onset of each mutagenesis experiment Shield1 (Clontech-631037) at 0.5 uM and dox (Clontech-631311) at 2 ug/ml were added to induce Cas9. To disrupt TP53, 3 TP53-targeting crRNAs each at 30 nM were delivered with 90 nM tracrRNA (IDT). Lipid delivery of synthetic crRNA/tracrRNAs is as described by Wells et al., 2016 (under review).

### Interactome Analysis

The Thomson Reuters' Computational Biology Methods for Drug Discovery (CBDD) toolkit, implements a number of published algorithms (in R) for network and pathway analysis of—omics data. An internal R wrapper functioned to facilitate the use of the CBDD toolkit to run the causal reasoning algorithm Chindelevitch et al. (2012) Bioinformatics 28:1114-1121, Jager et al. (2014) J. Biomol. Screen 19:191-802. The knowledgebase used was a combination of Thomson Reuters' MetaBase (a manually curated database of mammalian biology) and STRING Szklarczyk et al. (2015) Nucleic Acids Res. 43:D447-D452.

### OCT4 Targeting Assay

hPSCs were pre-treated with 1 uM thiazovivin for at least 2 hours and harvested using accutase. A mixture of 4 ug of Oct4-tdTomato-puroR targeting vector, 1 ug of each gRNA cloned into a vector that co-expresses Cas9-D10A (or a vector lacking gRNAs as a control), and 2 ug of either an episomal vector for p53DD (pCE-mP53DD) or EBNA1 alone (pCXB-EBNA1) were electroporated into 1×10̂6 cells using a Neon electroporation system (Thermo). Cells were deposited into one well of a 6-well dish coated with matrigel containing 50% fresh mTESR:50% conditioned mTESR supplemented with bFGF (10 ng/mL) and thiazovivin. After 48 hours, cells were selected with 0.3 ug/mL puromycin in the presence of thiazovivin.

CRISPR Indel Analysis from Genomic DNA

Genomic DNA was extracted from cells using the DNeasy Blood and Tissue Kit (Qiagen-69506) and automated sample prep was performed on the OIAcube (Qiagen). Each target was amplified using locus specific primers (Table S4). Indel analysis using NGS was as described by Wells et. al., 2016 (under review).

### LentICRISPR Transduction for Cas9 Mutagenesis

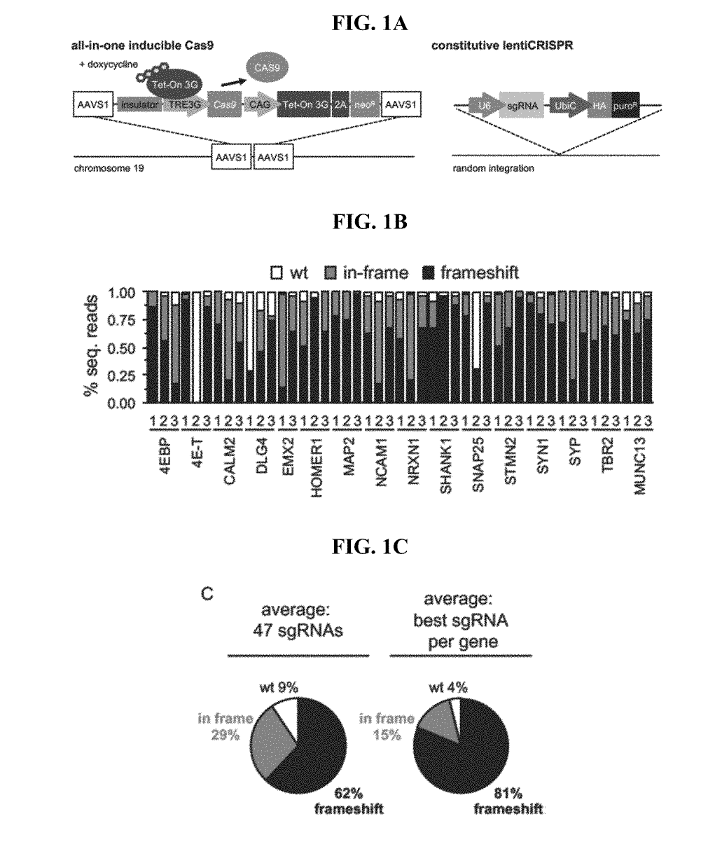

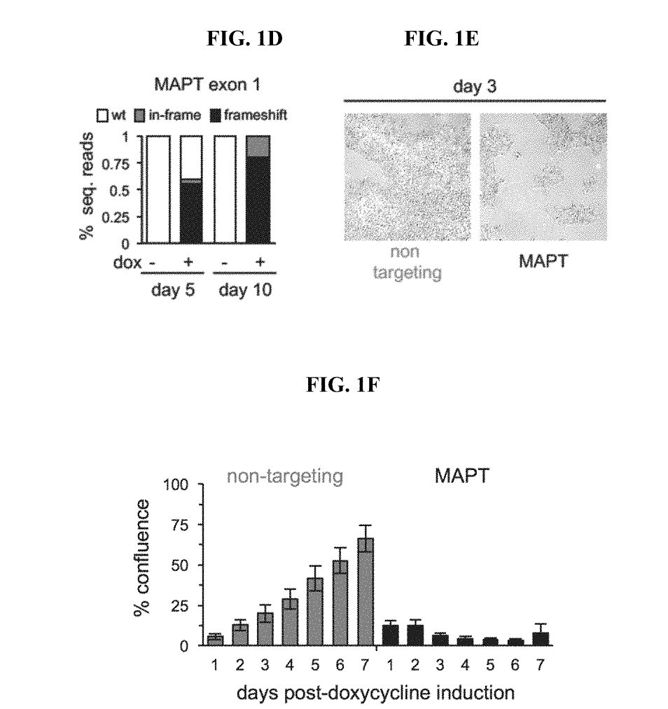

The 47 sgRNAs in FIG. 1 were designed using the sgRNA Designer (Broad Institute) and cloned into the pNGx_LV_g003_HA_Puro backbone by GenScript. The 13,000 lentiviral sgRNA library was designed, cloned into the pNGx_LV_g003_TagRFP_T2A_Puro backbone and packaged as described by Dejesus et al. (2016) eLife 5:1-16. For pooled screening viral titer was determined by exposing cells to a 12-point dose response of each lentiviral stock. 2*10⁵ cells were plated into a single well of a 6-well plate (2.1*10⁴ cells/cm²). Four days after infection % RFP was assayed by FACS (SONY SH800Z) and the data was used to calculate the amount of virus needed for 0.5 MOI. Puromycin concentration was optimized by infecting at 0.5 MOI and testing a dose-response of puro spanning 0.3 ug/ml to 2 ug/ml. At 2 ug/ml puromycin 100% of surviving cells are RFP positive.

### Pooled Screening

The 13,000 sub-genome library, included sgRNAs targeting 2600 genes and non-targeting controls, was designed, synthesized, cloned and packaged as described by Dejesus et al. (2016) eLife 5:1-16. To infect at 1000× coverage 5 T225 flasks were seeded at 2.1*10⁴ cells/cm² and infected at 0.5 MOI for each condition. 24 h after infection cells were treated with puromycin at concentration of 0.5 ug/ml for the remainder of the screen. Dox and Shield 1 were added to the Cas9 positive conditions from day 1 through day 12. At each passage cells were counted to maintain 1000× coverage for both the newly seeded flask and the pellet for DNA isolation. To generate log 2(fold change) values, DNA was isolated from pelleted cells which was PCR amplified with primers targeting the lentiviral sgRNA backbone. Next generation library construction, sequencing and data analysis was performed as described by Dejesus et al. (2016) eLife 5:1-16.

### Live Imaging of Confluency

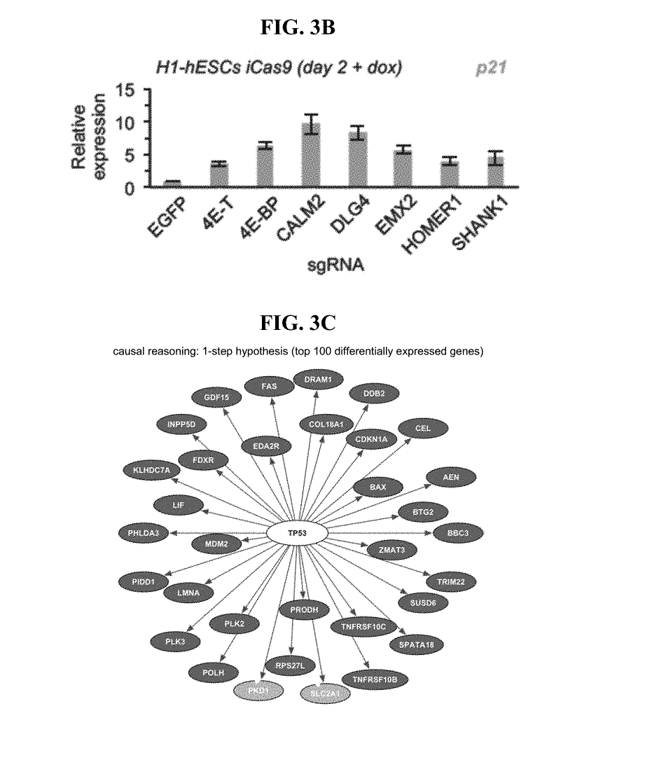

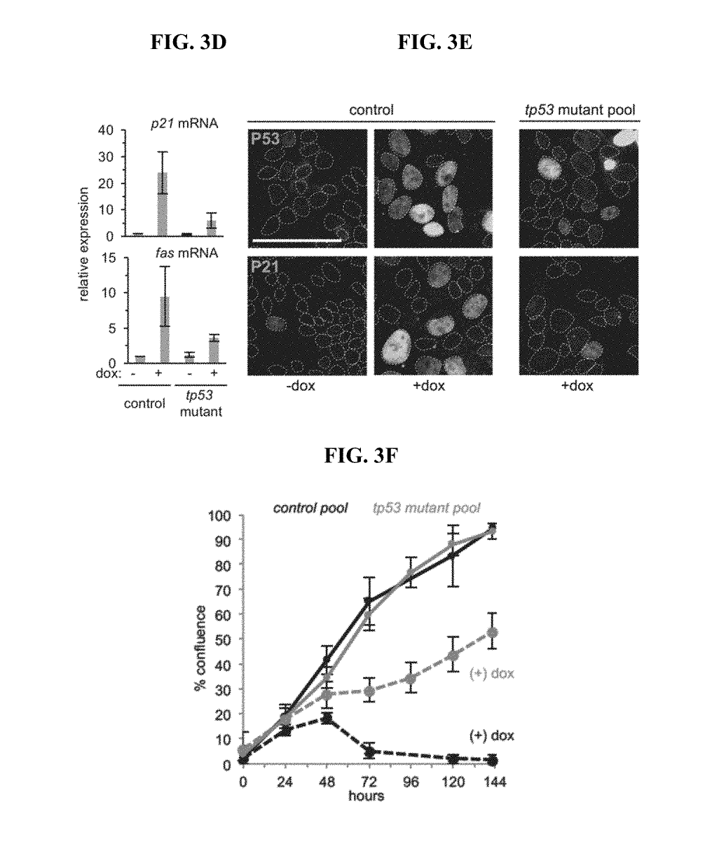

An IncuCyte zoom (Essen Biosciences) was used to quantify confluency in live cells each day post-media change. The confluence processing analysis tool (IncuCyte Zoom Software) calculated confluency for each sample. Average confluency and standard error was calculated for 96-well plates by taking a single image per well across multiple wells. Infected cells were allowed to recover after puro selection and were maintained in the absence of dox as to not induce mutations prior to the growth assay. For each population, cells were counted and plated in media containing dox at a density of 2.1*10⁴ cells/cm² on 96-well plates at the start of the experiment (day 0). For FIG. 1D 96 wells for non-targeting and 88 wells for MAPT were averaged. For FIG. 3F 24 wells were averaged for each condition.

RNA-seq, and qPCR

To detect signal from dying cells samples were collected by pelleting both the cellular debris in the media as well as the dissociated, formerly adherent, cells from an entire well per replicate in the same microcentrifuge tube. Total mRNA was isolated from using the RNeasy Mini kit plus (Qiagen-74134). The Agilent 2100 bioanalyzer and the Nano 6000 kit (Agilent-5067-1511) were used to quantify and check the quality of each mRNA sample. 240 ng of high quality RNA (RIN 10) was used for PolyA+ RNA-seq. Libraries were made using a Hamilton automated protocol with the TruSeq® Stranded mRNA LT sample prep kit (lllumina-RS-122-2101) and sequenced on the Illumina HiSeq 2500. An average of more than 50 million 76-bp paired-end reads was obtained per sample. Raw fastq files were aligned to a human reference genome (GRCh37.74) using the STAR aligner (v2.5.1b) Dobin et al. (2013) Bioinformatics 29(1): 15-21. Gene counts and transcript quantification values (TPM) was performed using HTSeq-count (v0.6.0) Anders et al. (2015) 31(2):166-9 and RSEM (v1.2.28) Li and Dewey (2011) BMC bioinformatics 12:323, respectively. The gene counts were then used for differential expression analysis using DESeq2 Love et al. (2014) Genome Biology 15(12):550. For qPCR mRNA concentration was measured using a Nanodrop 2000 (Thermo Scientific). 200 ug of RNA was used as template for cDNA synthesis using the SuperScript III first-strand synthesis system (ThermoFisher-18080051). cDNA was diluted 1:5 in H20 prior to analysis using taqman gene expression arrays and the 2× Fast Start Universal Probe master mix (ROX) (ROCHE-04913957001). 384-well qPCR plates were run on a ViiA 7 Real-Time PCR System (ThermoFisher). Relative expression was calculated as described by Pfaffl (2001) Nucleic Acids Research 29(9):e45. TaqMan gene expression arrays FAM-MGB (ThermoFisher-4331182); CDKN1A (Hs00355782_m1), bACTIN(Hs01060665_g1) fas (Hs00163653_m1). A custom TaqMan gene expression assay was ordered to detect Cas9 mRNA.

### Immunofluorescence and Microscopy

Cells were fixed in 4% PFA in PBS for 10 minutes at room temperature and were washed with 0.1% triton X-100 in PBS after fixation. Cells were blocked in 2% goat serum, 0.01% BSA and 0.1% triton X-100 in PBS for 1 hr at room temperature. Primary antibodies were diluted in blocking solution and incubated with cells over night at 4 C. Cells were washed 3 times before incubation with secondary antibodies or fluorescently conjugated primary antibodies at room temp for 1.5 hours. Cell were washed 3 times and incubated with DAPI 1:1000 for 5 minutes at room temp before imaging. Primary antibodies: 1:250 P21 (12D1) (CST-2947), 1:250 P53 (7F5) (CST-2527), 1:300 FLAG (M2) (Sigma-F1804). Secondary antibodies: 1:500 Goat anti-Mouse IgG (H+L) AF488 (ThermoFisher-A-11029), 1:500 Goat anti-Rabbit IgG (H+L) AF488 conjugate (ThermoFisher-A-11008). For OCT4 targeting assay live cells were imaged for tdTomato fluorescence and then fixed, permeabilized, washed incubated with peroxidase suppressor (Thermo) for 30 min, washed twice, and then blocked for 30 min (5% goat serum/0.1% Tween-20/PBS). Cells were incubated at 37 degrees for 2 hours with anti-TRA-1-60 (MAB4360, Millipore, 1:300 dilution), washed 3 times, and then for 1 hour with anti-IgM conjugated to HRP (31440, Thermo, 1:250). A metal enhanced DAB substrate kit was used for detection (34065, Thermo). Live and fixed immunofluorescent images were taken using the 10× and 20× objectives on an Axio Observer.D1 (Ziess). To quantify TP53 and P21 proteins using CellProfiler software, average immunofluorescent intensity was determined for each nucleus, and a positive-expression threshold was set based on the no-secondary control.

### Example 2: Cas9-Dependent Gene Disruption is Efficient and Toxic to hPSCs

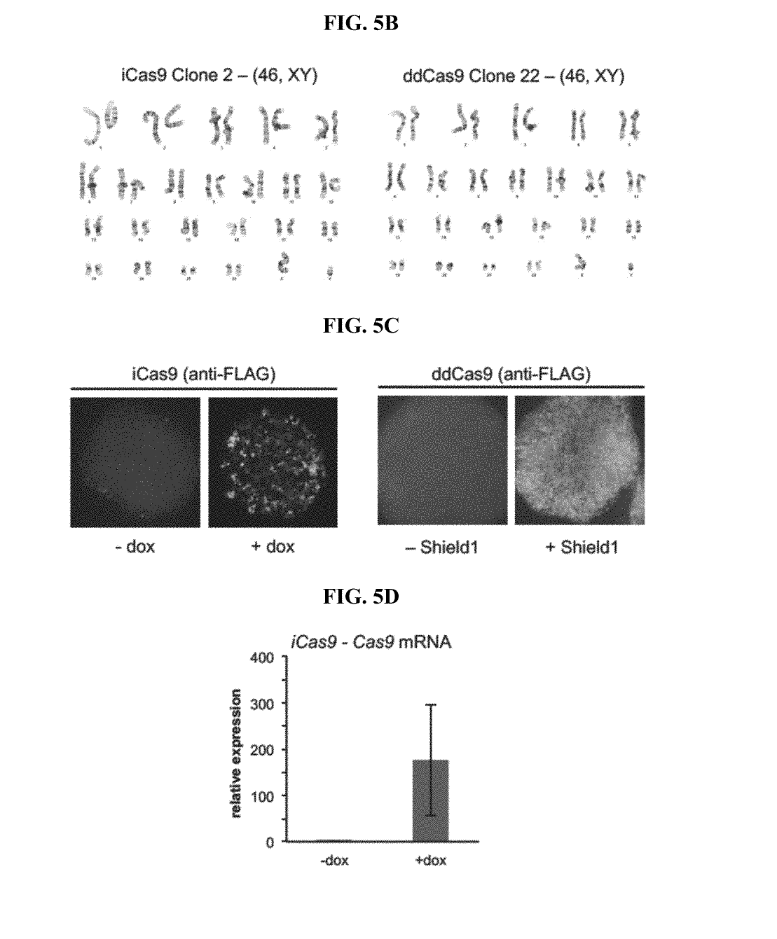

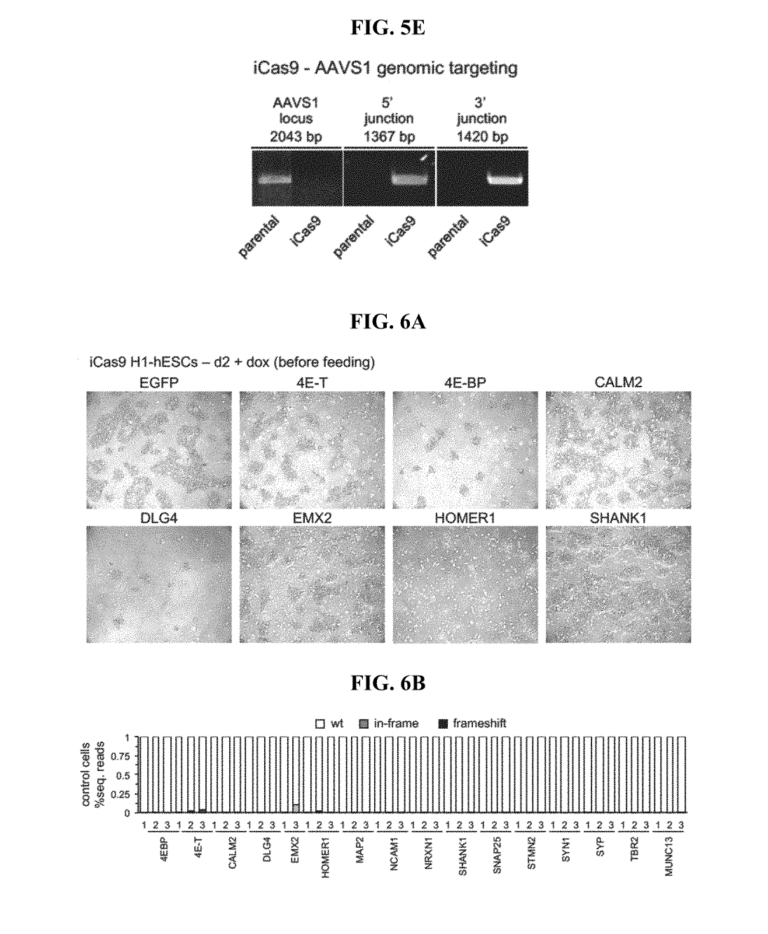

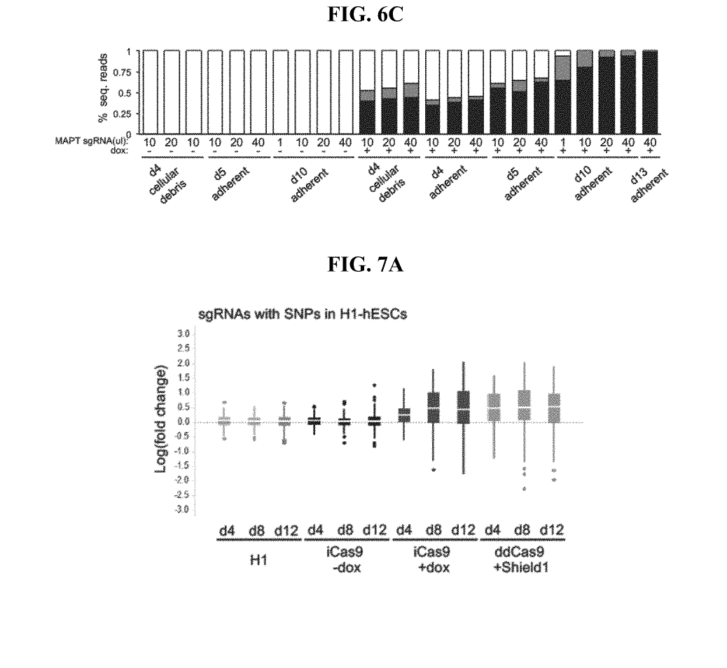

A two-component Cas9 system was developed to allow for rapid generation of mutant hPSCs. The system consists of a stable Cas9 line used with lentiviral sgRNAs (lentiCRISPR). The streamlined all-in-one doxycycline (dox) inducible Cas9 safe-harbor (AAVS1) construct, will be henceforth referred to as iCas9 The clone used for this study had a normal karyotype, strong induction of Cas9 in the presence of dox, and was property targeted (FIGS. 5A-5E). In order to test Cas9 activity, a next generation sequencing (NGS) pipeline to detect CRISPR indels was utilized to quantify control, in-frame and frameshift reads in DNA samples. iCas9 cells were infected with lentiCRISPRs targeting 47 loci in 16 genes and treated with dox for 8 days. NGS analysis of lentiCRISPR infected cells had high percentages of indels and demonstrates this is a significant improvement over existing systems in hPSCs (FIG. 1B) Gonzalez et al. (2014) Cell Stem Cell 15:215-226. The average editing for the 47 sgRNAs was over 90% (FIG. 1C). Picking 3 sgRNAs per gene identified on average at least 1 sgRNA generating over 80% loss-of-function alleles (FIG. 1C). hPSCs have a robust ability to repair Cas9-induced DSBs by NHEJ. Due to the high efficiency in generating indels it became evident there was a toxic response to Cas9-induced DSBs. The lentiCRISPRs impaired growth and increased cellular debris present in the media. This doubled the time to passage and created well-to-well variability amongst sgRNAs (FIGS. 6A-6C and FIG. 9 Table S1).

To investigate this more closely, we conducted follow-up studies with a lentiCRISPR targeting MAPT, a non-essential neuronal gene not expressed in hPSCs. Consistent with the 47 sgRNAs, 10 days of dox treatment completely edited the MAPT locus (FIG. 1D). Targeting MAPT severely reduced colony size relative to non-targeting controls cultured under the same conditions. (FIG. 1E). To quantify this, we measured confluency in live cells expressing either a non-targeting control or a MAPT targeting sgRNA. iCas9 cells were transduced with their respective lentiCRISPRs and plated at the same density in media containing dox (day 0). Live imaging each day demonstrated a marked difference in confluency (FIG. 1F). Confluency increased over 7 days in the control cells, in contrast, MAPT targeted cells exhibited a decrease in confluency despite being seeded at a similar density. To determine if this response was related to off-target DSBs, the top 6 predicted off targets identified by the CRISPR design tool Hsu et al. (2013) Nat. Biotechnol. 31:827-832, for the MAPT sgRNA were assayed by NGS (FIG. 6A-6C and Table S2). No predicted off-target mutations were identified. Although, this does exclude additional unpredicted off-target effects, the data suggests that hPSCs are extremely sensitive to a single or a few DSBs. Despite the apparent toxicity, gene editing efficiency was quite high and mutant cells recover making up for the initial loss if given enough time.

### Example 3: CRISPR Screens Identify hPSC-Specific Toxic Response to Cas9-Induced DSBs

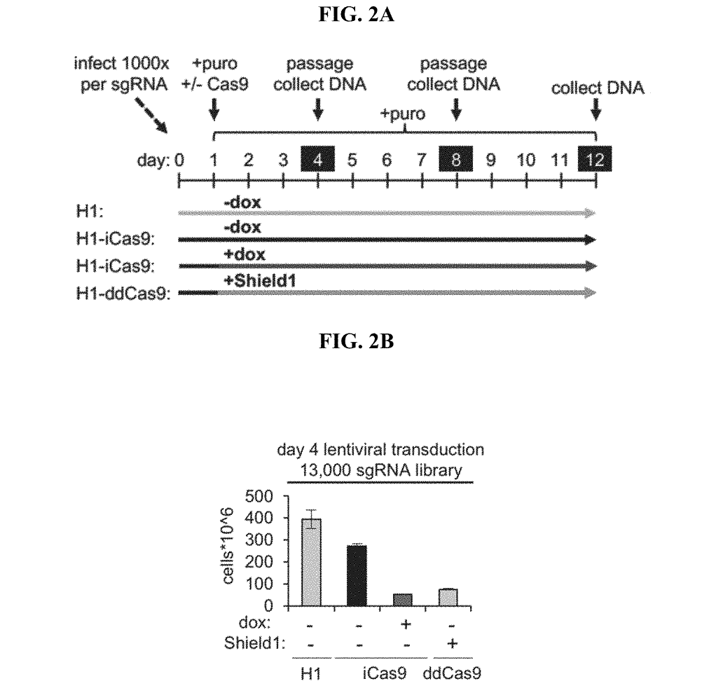

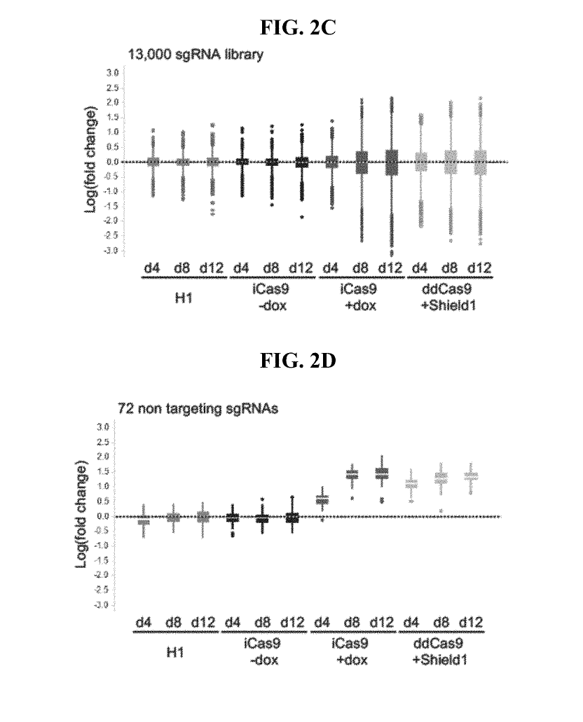

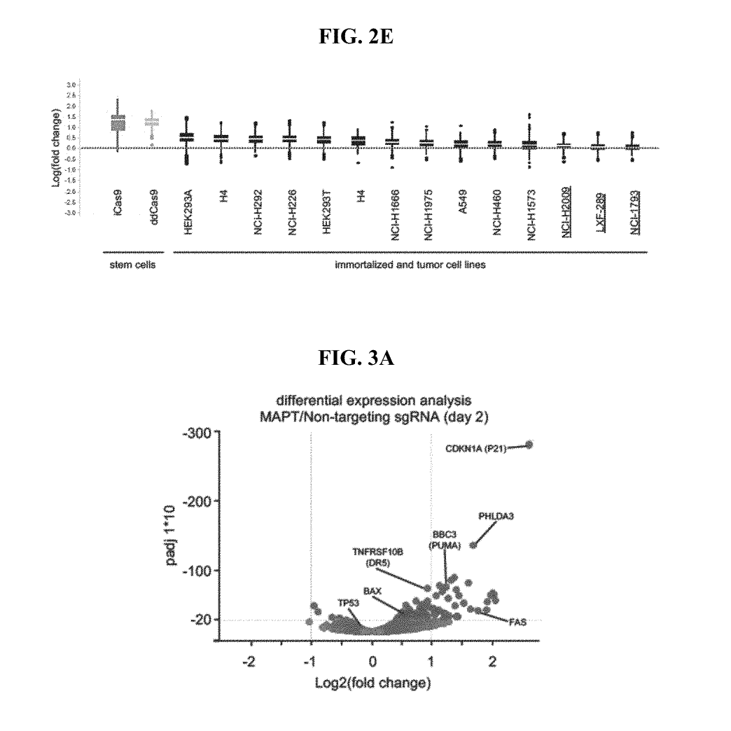

hPSCs expressing a non-targeting sgRNA have a proliferative advantage over cells expressing a targeting sgRNA. We hypothesized that non-targeting controls would increase representation when simultaneously tested with thousands of targeting sgRNAs in a pooled screen. To test this, we used a pooled sgRNA library consisting of 13,000 sgRNAs. 72 sgRNAs are non-targeting and the remaining target ˜2600 genes (5 sgRNAs/gene). 2000 cells for each sgRNA were infected at 0.5 MOI to maintain 1000× representation after puro selection (FIG. 2A). The experiment was highly controlled and four different conditions infected with the sgRNA library were tested. Two conditions were grown in the absence of Cas9 (−dox) and included the parental H1 and Hi-iCas9 cells. To further validate the toxicity and high editing efficiency in hPSCs we generated a second inducible Cas9 based on the Shield1-destabilizing domain (DD) system Banaszynski et al. (2016)Cell 126: 995-1004. His were generated with a Cas9 fused to a DD tag (ddCas9) which is stabilized in the presence of Shield1 and degraded in its absence (FIG. 5C). The remaining two conditions were grown in the presence of Cas9 induced by dox or Shield1 respectively. Cells were passaged every 4 days and a portion at 1000× was both replated or pelleted for DNA isolation. Cell counting at day 4 demonstrated that iCas9 or ddCas9 hESCs cultured with dox or Shield1 had little growth in comparison to H1 and iCas9 hPSCs infected with the same library, seeded at the same density, but in the absence of Cas9 (FIG. 2B).

A NGS pipeline was used to recover spacer sequences from cells infected with lentiCRISPRs by amplification and deep sequencing of the spacer sequence. All but one sample recovered 98% of expected spacer sequences and demonstrates adequate representation was maintained for all conditions. Fold change was calculated for each individual spacer sequence by dividing each condition by the initial representation in the lentiviral pool (FIG. 2C). In the Cas9 minus conditions sgRNAs remained distributed within +/−1 Log₂ (fold change) over the 12-day experiment. In contrast, the Cas9 positive conditions displayed a time-dependent change in sgRNA representation which increased the spread of the distribution. Plotting only the non-targeting controls identified a 1.2 to 1.4-fold enrichment specific to the Cas9 positive conditions (FIG. 2D).

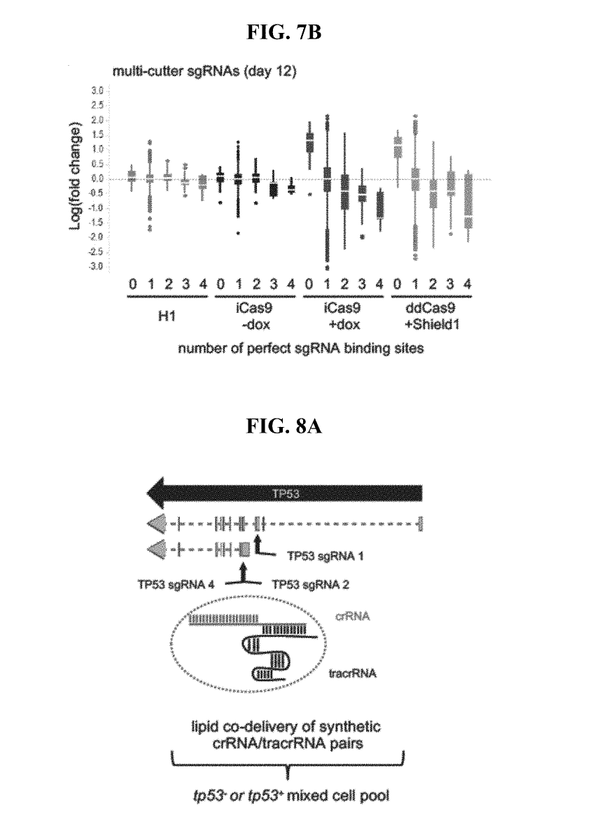

To determine if this toxic response is specific to hESCs we evaluated the non-targeting controls across pooled CRISPR screens in other cell lines as marker for sensitivity to DSBs. Fold change was calculated for non-targeting sgRNAs from 14 additional transformed cell lines using a genome-wide version of the hPSC library. Plotting the same day 12 non-targeting controls from the Cas9 positive conditions from FIG. 2B with the transformed lines demonstrated a heightened sensitivity to DSBs in hPSCs (FIG. 2E). hPSCs have a greater than 1.20 log₂ (fold change) while transformed cell lines show little enrichment (0.51 to 0.05 log₂ (fold change)). Lastly, we exploited design flaws effecting a subset of the sgRNA library to identify additional evidence for DSB-toxicity. SNPs present in the H1-hESC genome disrupted 1.9% of the sgRNAs causing them to significantly enrich (FIGS. 7A-7B). Multiple perfect cut sites were identified for 1.1% of the sgRNAs causing a dose-dependent depletion of multi-cutters (FIGS. 7A-7B). Cumulatively, these results demonstrate that hPSCs are extremely sensitive to DSBs induced by Cas9 and this negative effect presents a significant challenge for both engineering and screening efforts.

### Example 4: Cas9-Induced DSBs Trigger a TP53-Dependent Toxic Response in hPSCs

To further investigate the acute response to DSBs in hPSCs, RNA-seq was performed on samples at the onset of the toxic response. iCas9 cells infected with a non-targeting control or MAPT sgRNA were grown in the presence of dox for 2 days. We generated a volcano plot of differentially expressed genes to display statistical significance (adjusted p-value) against log₂(fold change) (FIG. 3A, Table S3). The distribution of values with high statistical significance was skewed overall with the presence of a DSB increasing the expression of response genes. Consistent with this toxic response, gene ontology analysis of the top 100 hits identified 25 genes with roles in, programmed cell death and apoptosis (STRING-db, FDR 1.92E-08). Notably components of the intrinsic and extrinsic cell death pathways such as BAX, BBC3, FAS, and TNFRSF10B were upregulated in cells with DSBs. The most differentially expressed gene was CDKN1A(p21) (6.12 fold, 6.6E-298 padj) a cell cycle regulator with known roles in DDR Cazzalini et al. (2010) Mutat. Res. 704:12-20. To confirm these results qPCR measured p21 mRNA levels from 7 independent sgRNAs 2 days after dox induction (FIG. 3B). The expression for each targeting sgRNAs was normalized to the EGFP control and demonstrated a 3 to 10-fold increase in p21 mRNA. Consistent with the induction of p21, these 7 sgRNAs exhibited a toxic response with reduced growth and increased cellular debris in the media (FIGS. 6A-6C).

To identify key pathways involved an in-silico interactome analysis was performed on the top 100 differentially expressed genes (adjusted p-value cutoff of 1.2E-17). A number of the causal reasoning algorithms consistently identified TP53 as one of the top ranking hypotheses along with MYC, SP1 and EP300 Chindelevitch et al. (2012) Bioinformatics 28:1114-1121, Jager et al. (2014) J. Biomol. Screen 19:191-802. All these hypotheses are tightly interconnected. Further investigation was focused on TP53 because of its well-established role in the DDR Lane (1992) Nature 358:15-16. The 1-step TP53 hypothesis accurately explains 33 out of the 100 input genes (FIG. 3C) and is fitting considering the most differentially expressed gene was p21, a canonical TP53 target El-Deiry et al. (1993) Cell 75:817-825. In further support, examining the TP53 mutation status in the transformed lines identified that 3 lines with the least enrichment of non-targeting sgRNAs have TP53 mutations (FIG. 2E, far right). Initially the involvement of the TP53 was obscured because differential expression revealed no significant change in TP53 mRNA. Consistent with this, it is well documented that TP53 activity and expression are regulated post-transcriptionally Canman et al. (1998) Science 281:1677-1679, Vassilev et al. (2004) Science 303: 844-848.

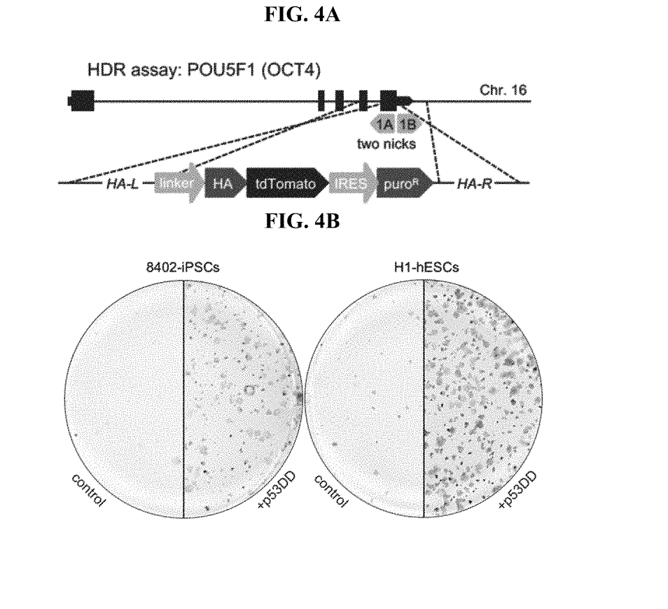

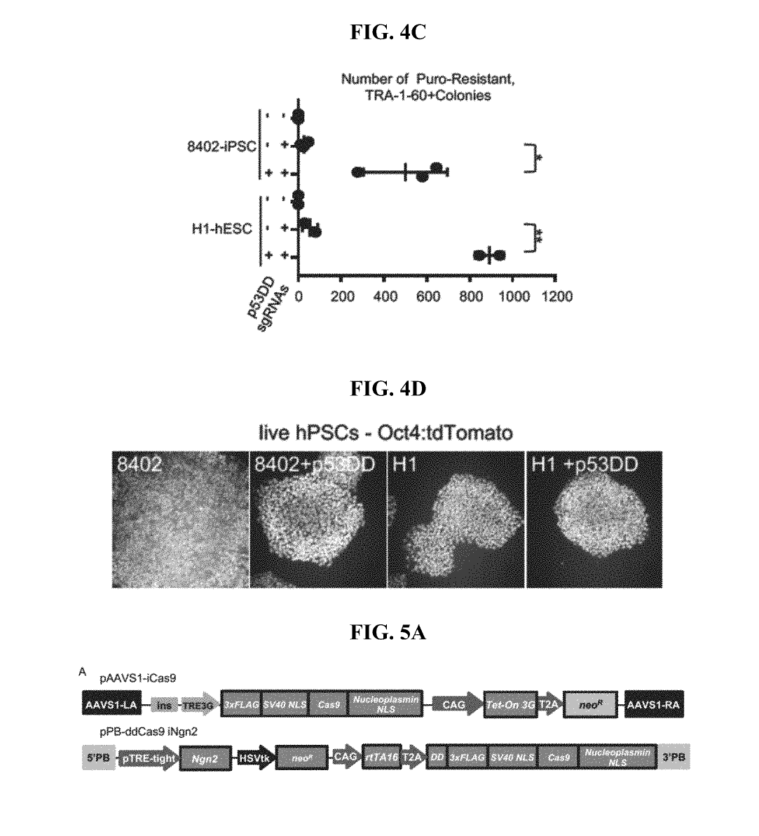

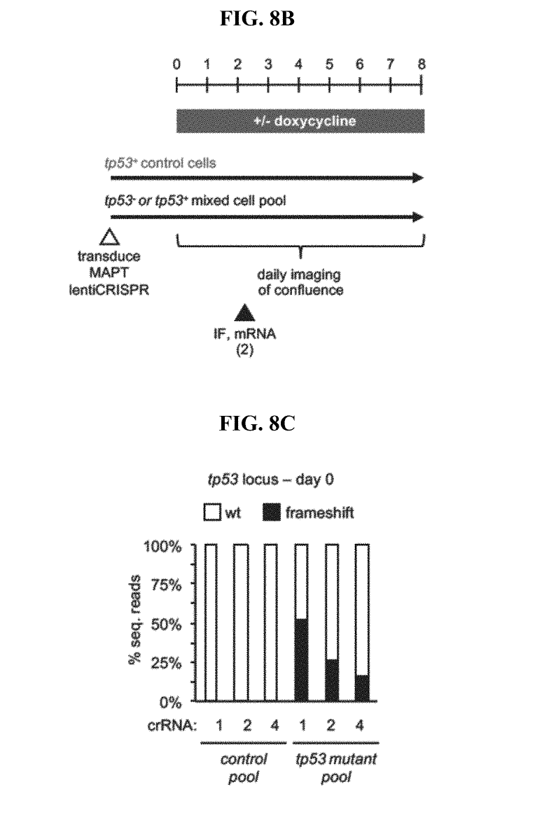

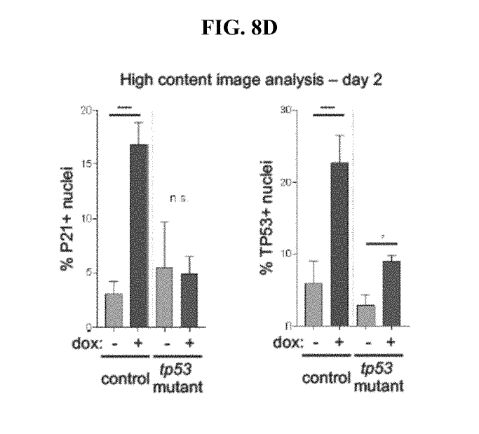

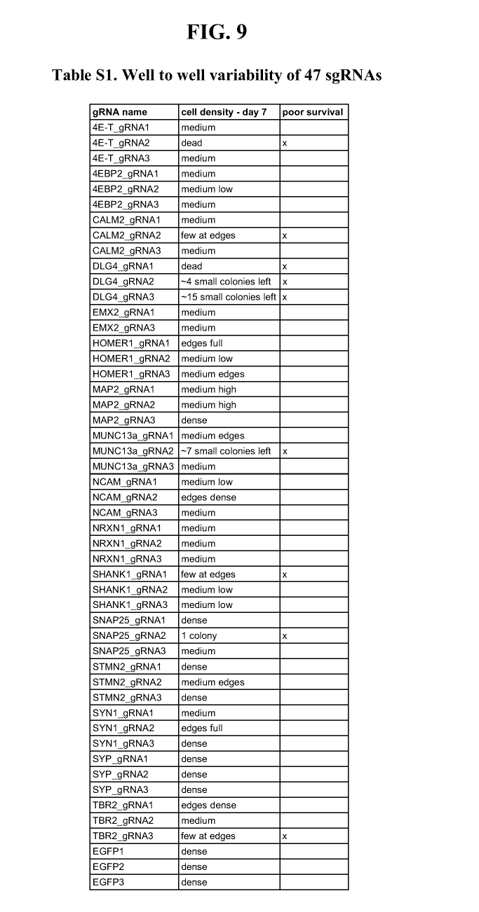

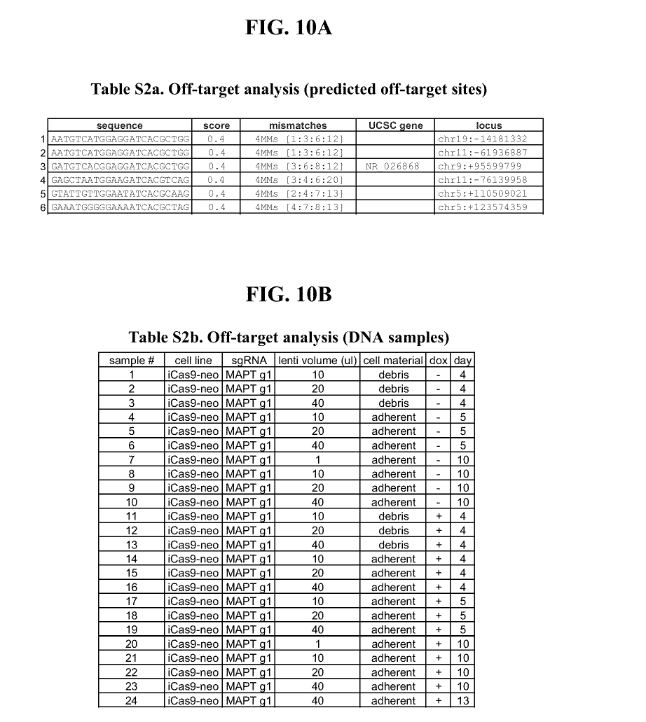

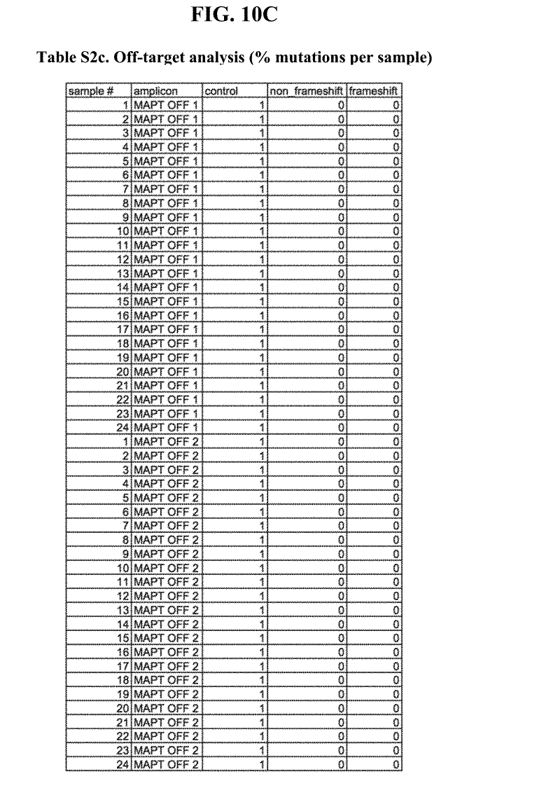

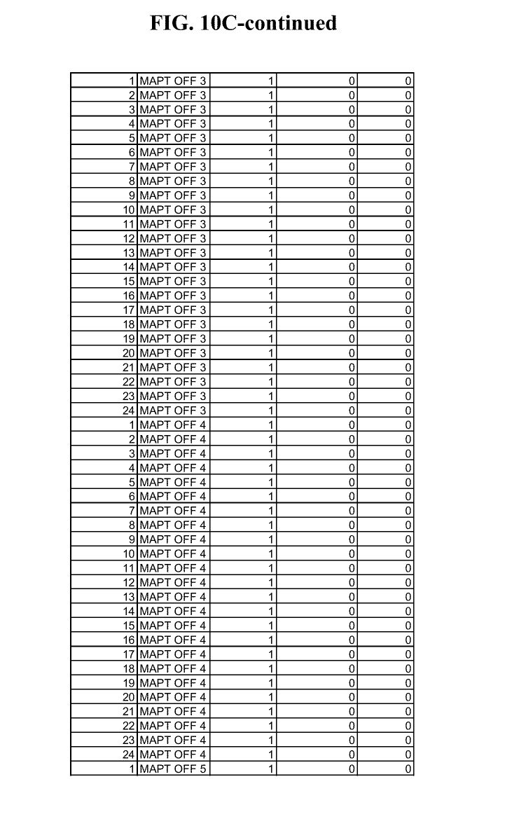

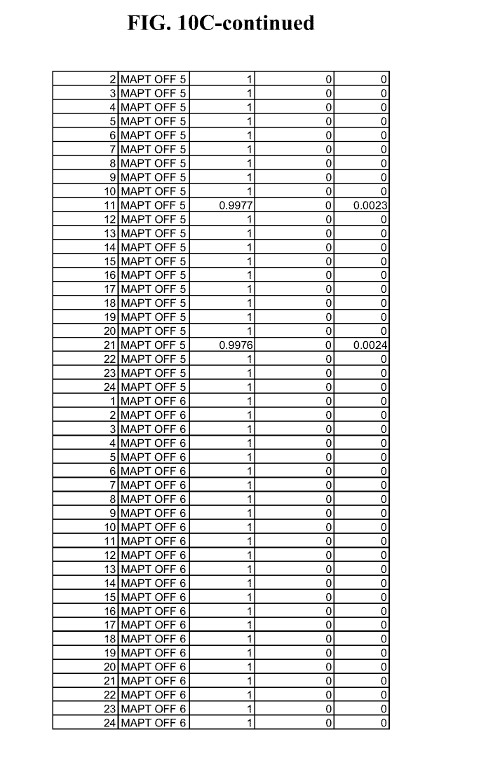

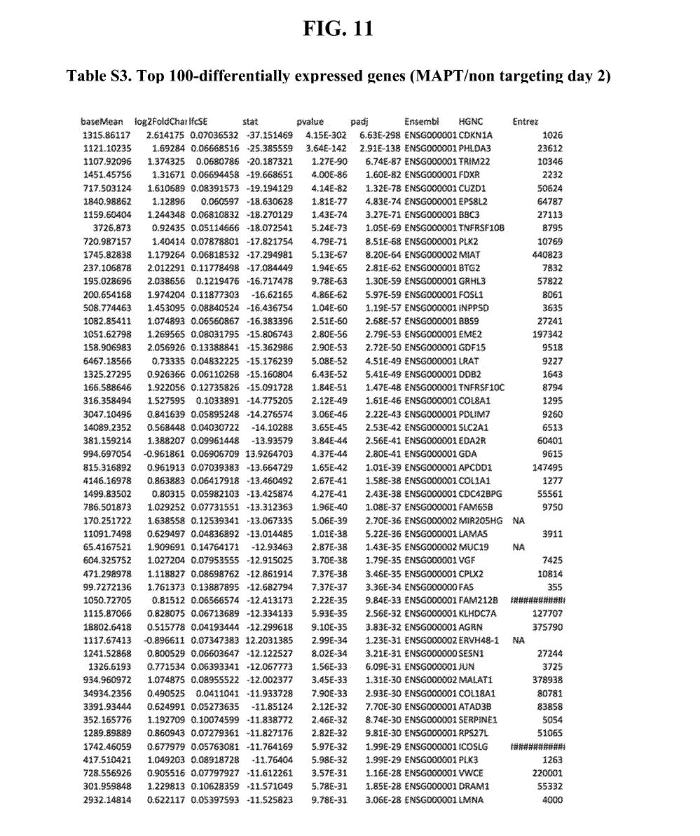

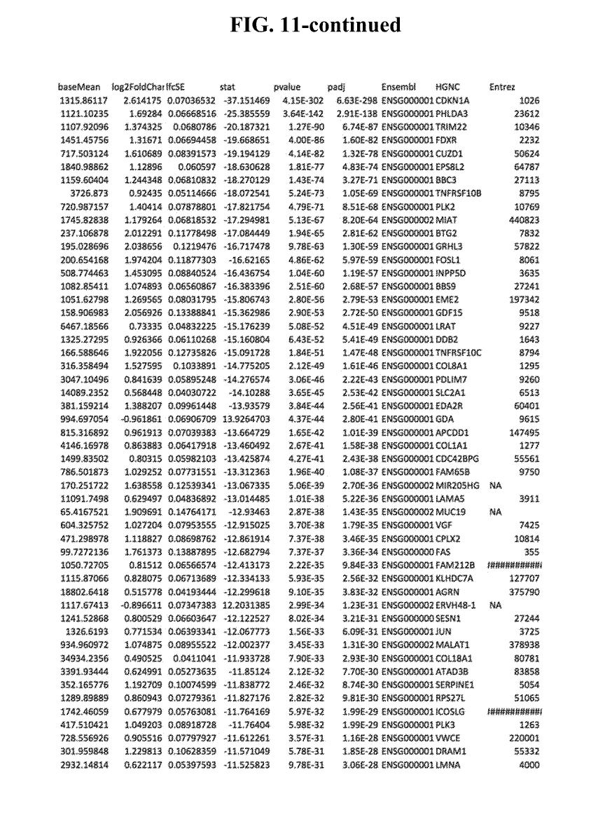

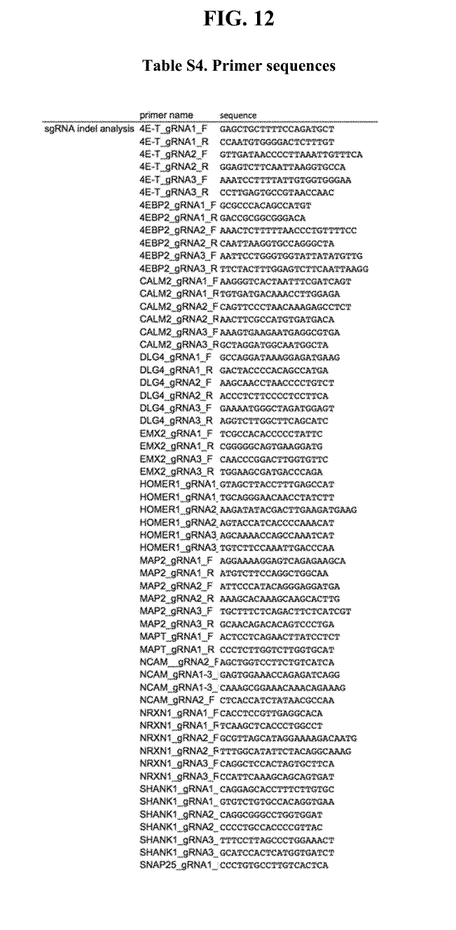

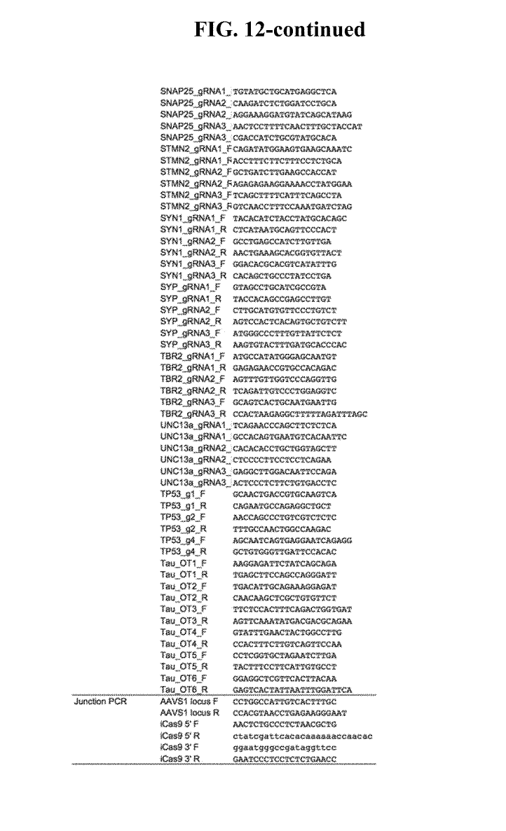

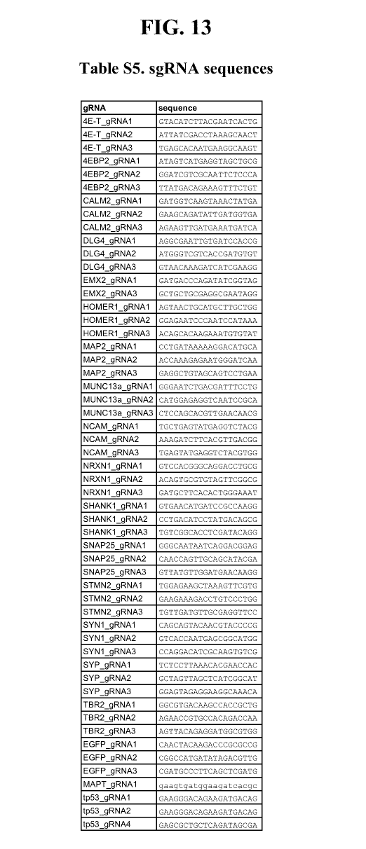

Interactome analysis implied that TP53 was responsible for the toxic response. To demonstrate that TP53 is functionally involved, a TP53 mutant pool was rapidly generated by transiently transfecting 3 synthetic crRNA/tracrRNA pairs targeting the TP53 locus in dox treated iCas9 cells (FIGS. 8A-8D). The resulting mutant pool had a mixture of over 50% frameshift mutations at 3 independent sites within the TP53 ORF (FIGS. 8A-8D). The control pool and TP53 mutant pool were subjected to Cas9 mutagenesis using the MAPT sgRNA. Infected cells of both genotype were grown +/− dox for up to 6 days. To confirm that the transcriptional response to DSBs is TP53-dependent, mRNA was isolated at day 2 and quantified using qPCR (FIG. 4D). Control cells exhibited a strong induction of p21 and fas mRNA that was significantly muted in the TP53 mutant pool. To confirm the involvement of TP53 and P21 at the protein level immunofluorescence was conducted at day 2. In response to a DSB both TP53 and P21 increase (FIG. 3E, FIGS. 8A-8D). This increase was not detected in the TP53 mutant pool (FIG. 3E, FIGS. 8A-8D). Lastly, confluency was measured for 6 days in control and the TP53 mutant pool. TP53 mutants grown in the presence of dox continue to grow while dox treated controls die (FIG. 3F).

Collectively these results demonstrate that TP53 is required for the toxic response to DSBs induced by Cas9 induced.

### Example 5: TP53 Inhibition Enhances Cas9 Genome Engineering in hPSCs

It is possible that DNA damage-induced toxicity, in hPSCs, is a significant challenge to precise engineering using DSBs to stimulate HDR. To test if blocking TP53 results in increased HDR efficiency, we developed an assay based on precise targeting of the oct4/POU5F1 locus. To increase the specificity of DSB induction a pair of dual nickases Ran et al. (2013) Cell 154:1380-1389 flanking the stop codon were used to initiate HDR with a gene trapping plasmid (FIG. 4A). The gene trap does not contain a promoter or nuclear localization signal of its own and only correctly targeted cells will express a nuclear tdTomato and gain resistance to puromycin. TP53 signaling was blocked by using a overexpression construct (p53DD) that has been used to increase reprograming efficiency of iPSCs Hong et al. (2009) Nature 460: 1132-1135, Schlaeger et al. (2015) Nature Biotechnol. 33:58-56. The Cas9^{D10A}-gRNA(s) and gene trapping oct4 plasmids were co-electroporated with or without the p53DD plasmid and scored for the number of puromycin-resistant colonies expressing nuclear tdTomato (FIGS. 4B-4D). TP53 inhibition greatly increased the number and size of TRA-1-60 positive colonies surviving the engineering and selection process in both 8402-iPSCs and H1-hESCs (FIG. 4B). Quantification by counting colonies from independent experiments showed that control 8402-iPSCs and H1-hESCs had an average of 26.3 and 54.5 colonies and that p53DD significantly boosted this average to 500 and 892 respectively (FIG. 4C). TP53 inhibition increased the efficiency of HDR resulting in a 19-fold increase for 8402-iPSCs and a 16-fold increase for H1-hESCs. TP53 inhibition dramatically improves engineering in hPSCs and confirms TP53 is a major barrier to precise engineering in cells with an intact DDR.

Targeted genome engineering using Cas9 has significant therapeutic potential; however, to exploit this we must be able to modify efficiently without creating undue stress in many cell types. We developed a highly efficient Cas9 system in hPSCs. To our surprise, DSBs induced by Cas9 trigger a toxic response. Recently a few groups demonstrated that transformed cell lines are sensitive to multiple cuts induced by Cas9 Aguire et al. (2016) Cancer Discov. 6(8): 915-929, Hart et al. (2015) Cell 163: 1-12, Munoz et al. (2016) Cancer Discov. 6(8): 901-913, Wang et al. (2015) Science 350(6264: 1096-1101. In contrast, a single cut is sufficient to kill the majority of hPSCs. Given their biological similarity to the early embryo it is fitting that hPSCs are intolerant of DNA damage. The extreme sensitivity to DSBs may be a control mechanism to remove aberrant cells from developing into an organism Dumitru et al. (2012) Mol. Cell 46: 573-583, Liu et al. (2013) 13: 483-491. Steps can be taken to curb the effects of DSB toxicity in sensitive cells, such as using more cells, bioinformatics approaches and secondary screens to identify DSB related artifacts, genome specific sgRNA design, Cas9 variants with no off-targets, and TP53 inhibition.

Cas9 toxicity has not been reported and it was unclear why hPSCs were responding negatively. RNA-seq, computational analysis, and TP53 inhibition experiments demonstrated that the TP53 pathway is responsible for DSB-induced toxicity. The toxic response provides an explanation for the long-standing observation that hPSCs have inefficient rates of genome-engineering. By definition TP53 function is diametrically opposed to genome engineering. A study comparing HDR efficiencies across cell lines identified a 10- to 20-fold reduction in hPSCs relative to transformed lines He et al. (2016) Nucleic Acids Res. 44:1-14. These results are in agreement with our observation that TP53 inhibition increases HDR efficiency by an average of 17-fold in hPSCs. Engineering using TP53 inhibition greatly accelerates the process taking only a week whereas the standard approach takes a month to generate a working amount of cells. While long-term TP53 inhibition can lead to increased mutational burden, transient inhibition appears to be tolerated in hPSCs Hanel and Moll (2012) 113: 433-439, Schlaeger et al. (2015) Nature Biotechnol. 33:58-56.

The toxic response to Cas9 has important implications for gene therapy. To treat diseases of hard-to-isolate primary cells with a functional DDR the increase in efficiency from transient TP53 inhibition may be critical for success. It may be safer to transiently inhibit TP53 when engineering for therapies in cells with a heightened DDR. TP53 mutants continue to proliferate despite the presence of DSBs giving them a selective advantage. Transient inhibition of TP53 would reduce the selection pressure for preexisting or spontaneously induced TP53 mutations. Before engineering patient cells, the risks and benefits must be fully evaluated. It will be imperative to determine the spontaneous mutation rate of TP53 in engineered cells as well as the mutational burden associated with transient TP53 inhibition. As gene and cell therapies become tangible it will be critical to ensure patient cells have a functional TP53 before and after engineering.

Unless defined otherwise, the technical and scientific terms used herein have the same meaning as they usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. A gene editing system comprising an apoptosis inhibitor.

2. The gene editing system of claim 1, wherein said apoptosis inhibitor is a TP53 inhibitor.

3. The gene editing system of claim 1 further comprising a nuclease or a gene editing vector.

4. The gene editing system of claim 1, wherein the gene editing system comprising:
a TP53 inhibitor, and
a nuclease.

5. The gene editing system of claim 1, wherein the gene editing system comprising:
a TP53 inhibitor, and
a gene editing vector.

6. The gene editing system of claim 1, further comprising a growth factor.

7. The gene editing system of claim 3, wherein said nuclease is a meganuclease, zinc finger nuclease (ZFNs), transcription activator-like effector-based nuclease (TALEN), CPF1, or Cas9.

8. The gene editing system of claim 1, wherein said gene editing system is a Cas9 system that comprises:
a TP53 inhibitor,
a Cas9 molecule, and
a gRNA molecule, wherein the gRNA molecule is capable of targeting the Cas9 molecule to a target nucleic acid.

9. The gene editing system of claim 2, wherein the TP53 inhibitor is a protein, a nucleic acid, an antibody, a small molecule, or a gene editing system (e.g., dCas9-transcription repressor fusion) that targets TP53 and inhibits its function.

10. The gene editing system of claim 2, wherein the TP 53 inhibitor is a protein, a nucleic acid, an antibody, a small molecule or a gene editing system (e.g., Cas9 fusion to active MDM2) that targets MDM2 and activates its function.

11. The gene editing system of claim 9, wherein said TP53 inhibitor is a nucleic acid, and wherein said nucleic acid is a DNA, mRNA, siRNA, a shRNA, a miRNA, an antiMiR or an aptamer.

12. The gene editing system of claim 11, wherein said nucleic acid comprises SEQ ID NO: 9.

13. The gene editing system of claim 9, wherein said TP53 inhibitor is a protein, and wherein said protein is a TP53 variant that inhibits naturally occurring TP53 expression.

14. The gene editing system of claim 13, wherein said TP53 variant comprises SEQ ID NO:6, SEQ ID NO:7, or SEQ ID NO:8.

15. The gene editing system of claim 9, wherein said TP53 inhibitor is a small molecule that is pifithrin-alpha or pifithrin-mu.

16. The gene editing system of claim 8, wherein said Cas9 system further comprises a second gRNA molecule, and wherein the second gRNA molecule is capable of targeting the Cas9 molecule to the target nucleic acid.

17. The gene editing system of claim 8, wherein said gRNA molecule is an RNA molecule, or a DNA molecule encoding the gRNA molecule.

18. The gene editing system of claim 8, wherein the Cas9 molecule is a Cas9 polypeptide or a nucleic acid encoding a Cas9 polypeptide.

19. The gene editing system of claim 18, wherein the Cas9 molecule is a wildtype Cas9 molecule of S. pyogenes.

20. The gene editing system of claim 18, wherein the Cas9 molecule comprises one or more mutations as compared to a wild type Cas9.

21. The gene editing system of claim 8, wherein expression of said Cas9 molecule is regulated.

22. The gene editing system of claim 21, wherein the expression of said Cas9 molecule is induced by using doxycycline, shield 1, 4HT, rapamycin, or Light.

23. The gene editing system of claim 21, wherein the expression of said Cas9 molecule is inhibited by using ASV/CLV SMASHTAG.

24. The gene editing system of claim 5, wherein said gene editing vector is a recombinant adeno-associated virus (rAAV) based gene editing vector.

25. The gene editing system of claim 1, further comprising a template nucleic acid.

26. The gene editing system of claim 25, wherein the template nucleic acid comprises a circular nucleic acid.

27. The gene editing system of claim 26, wherein the circular nucleic acid is a plasmid.

28. The gene editing system of claim 25, wherein the template nucleic acid is a linear nucleic acid.

29. The gene editing system of claim 25, wherein the template nucleic acid comprises a double strand sequence.

30. The gene editing system of claim 25, wherein the template nucleic acid comprises a single strand oligonucleotide.

31. A cell comprising the gene editing system of claim 1.

32. The cell of claim 31, wherein said cell is a cell from a human.

33. The cell of claim 31, wherein said cell is a cell from a non-human subject.

34. The cell of claim 33, wherein said subject is a pig.

35. The cell of claim 31, wherein said cell is further engineered to express a chimeric antigen receptor (CAR).

36. A composition comprising the gene editing system of claim 1.

37. A pharmaceutical composition comprising the composition of claim 36 and a pharmaceutically acceptable carrier.

38. A kit comprising the gene editing system of claim 1.

39. The pharmaceutical composition of claim 38, further comprising instructions for use to treat a disorder.

40. A vector comprising the gene editing system of claim 1, or components thereof.

41. The vector of claim 40, wherein said vector is a viral vector.

42. The vector of claim 40, wherein said vector is an AAV vector or a lentiviral vector, wherein when the gene editing system comprises a rAAV based gene editing vector, said vector of claim 40 is an AAV vector.

43. A method of altering the structure of a cell comprising contacting the cell with:
the gene editing system of claim 1,
under conditions that allow for alteration of the structure of the cell, thereby altering the structure of the cell.

44. The method of claim 43, wherein the structure of the cell is altered by altering the sequence of the target nucleic acid in the cell.

45. A method of treating a subject by altering the structure of a cell in the subject, comprising contacting the cell with:
the gene editing system of claim 1,
under conditions that allow for alteration of the structure of the cell, thereby treating the subject by altering the structure of the cell in the subject.

46. A method of decreasing toxicity or promoting DNA repair of a break in a nucleic acid in a cell via an HDR pathway, the method comprising contacting the cell with:
the gene editing system of claim 1,
under conditions that allow for alteration of the structure of the cell, thereby treating the subject by altering the structure of the cell in the subject.

47. The method of claim 43, wherein said cell is a cell from a human.

48. The method of claim 43, wherein said cell is a cell from a non-human subject.

49. The method of claim 48, wherein said subject is a pig.

50. The method of claim 43, wherein TP35 inhibition is transient.

51. The method of claim 43, wherein the cell is contacted with a TP53 inhibitor after being contacted with the nuclease (e.g., Cas9).

52. The method of claim 43, wherein the cell is contacted with a TP53 inhibitor before being contacted with the nuclease (e.g., Cas9).

53. The method of claim 43, wherein the cell is contacted with the TP53 inhibitor and the nuclease (e.g., Cas9) at the same time.

54. The method of claim 43, wherein the target nucleic acid is altered to comprise the sequence of at least a portion of a template nucleic acid.

55. The method of claim 45, wherein the subject has a disorder that is caused by a mutation in the target nucleic acid.

56. The method of claim 55, wherein the disorder is cancer, a genetic disease, an infectious disease, a disorder caused by aberrant mitochondrial DNA (mtDNA), a metabolic disease, a disorder caused by aberrant cell cycle, a disorder caused by aberrant angiogenesis, a disorder caused by aberrant DNA damage repair, or a pain disorder.

57. The method of claim 43, wherein the cell is modified ex vivo.

58. A method of decreasing toxicity of gene editing to a cell comprising contacting said cell with an apoptosis inhibitor.

59. A method of modifying a donor cell or organ for transplantation comprising contacting said donor cell or organ with an apoptosis inhibitor, and performing gene editing to said donor cell or organ.

60. The method of claim 59, wherein said apoptosis inhibitor is a TP53 inhibitor.

61. The method of claim 59, wherein said gene editing uses a nuclease, and wherein said nuclease is a meganuclease, zinc finger nuclease (ZFNs), transcription activator-like effector-based nuclease (TALEN), CPF1, or Cas9.

62. The method of claim 59, wherein said gene editing is a targeted gene editing using a viral vector.

63. The method of claim 63, wherein said viral vector is a recombinant AAV Clade F vector.

64. The method of claim 59 further comprising contacting the cell with growth factor, e.g., basic fibroblast growth factor (bFGF).

65. The method of claim 59, wherein said donor is a non-human subject.

66. The method of claim 65, wherein said subject is a pig.

67. The method of claim 66, wherein said gene editing system is used to inactivate a porcine endogenous retrovirus (PERV).

68. The method of claim 60, wherein the TP53 inhibitor is a protein, a nucleic acid, an antibody, a small molecule, or a gene editing system (e.g., dCas9-transcription repressor fusion) that targets TP53 and inhibits its function.

69. The method of claim 60, wherein the TP 53 inhibitor is a protein, a nucleic acid, an antibody, a small molecule or a gene editing system (e.g., Cas9 fusion to active MDM2) that targets MDM2 and activates its function.


SEQUENCE LISTING (209 sequences)

SEQ ID NO: 1 - protein, 7 aa, Simian virus 40
PKKKRKV

SEQ ID NO: 2 - RNA, 42 nt, Artificial Sequence
  Description of Artificial Sequence Synthetic oligonucleotide
  modified_base (1)..(20): a, c, u, g, unknown or other
nnnnnnnnnnnnnnnnnnnnguuuuagagcuaugcuguuuug

SEQ ID NO: 3 - RNA, 86 nt, Artificial Sequence
  Description of Artificial Sequence Synthetic oligonucleotide
  misc_feature (80)..(86): May or may not be present
aacuuaccaaggaacagcauagcaaguuaaaauaaggcuaguccguuaucaacuugaaaaaguggcaccgagucggugcuuuuuuu

SEQ ID NO: 4 - RNA, 74 nt, Artificial Sequence
  Description of Artificial Sequence Synthetic oligonucleotide
  misc_feature (68)..(74): May or may not be present
aacagcauagcaaguuaaaauaaggcuaguccguuaucaacuugaaaaaguggcaccgagucggugcuuuuuuu

SEQ ID NO: 5 - RNA, 103 nt, Artificial Sequence
  Description of Artificial Sequence Synthetic polynucleotide
  modified_base (1)..(20): a, c, u, g, unknown or other
  misc_feature (97)..(103): May or may not be present
nnnnnnnnnnnnnnnnnnnnguuuuagagcuagaaauagcaaguuaaaauaaggcuaguccguuaucaacuugaaaaaguggcaccgagucggugcuuuuuuu

SEQ ID NO: 6 - protein, 105 aa, Artificial Sequence
  Description of Artificial Sequence Synthetic polypeptide
MEEPQSDPSVEPPLSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 7 - protein, 106 aa, Artificial Sequence
  Description of Artificial Sequence Synthetic polypeptide
MASMTGGQQMGSPPGSTKRALPNNTSSSPQPKKKPLDGEYFTLQIRGRERFEMFRELNEALELKDAQAGKEPGGSRAHSSHLKSKKGQSTSRHKKHMFKTEGPDSD

SEQ ID NO: 8 - protein, 102 aa, Artificial Sequence
  Description of Artificial Sequence Synthetic polypeptide
MTAMEESQSDISLKRALPTCTSASPPQKKKPLDGEYFTLKIRGRKRFEMFRELNEALELKDAHATEESGDSRAHSSYLKTKKGQSTSRHKKTMVKKVGPDSD

SEQ ID NO: 9 - DNA, 309 bp, Artificial Sequence
  Description of Artificial Sequence Synthetic polynucleotide
atgactgccatggaggagtcacagtcggatatcagcctcaagagagcgctgcccacctgcacaagcgcctctcccccgcaaaagaaaaaaccacttgatggagagtatttcaccctcaagatccgcgggcgtaaacgcttcgagatgttccgggagctgaatgaggccttagagttaaaggatgcccatgctacagaggagtctggagacagcagggctcactccagctacctgaagaccaagaagggccagtctacttcccgccataaaaaaacaatggtcaagaaagtggggcctgactcagactga

SEQ ID NO: 10 - DNA, 10 bp, Artificial Sequence
  Description of Artificial Sequence Synthetic oligonucleotide
rrrgwwcyyy

SEQ ID NO: 11 - DNA, 145 bp, Adeno-associated virus
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 12 - DNA, 145 bp, Adeno-associated virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 13 - DNA, 167 bp, Adeno-associated virus
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 14 - DNA, 167 bp, Adeno-associated virus
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 15 - protein, 491 aa, Artificial Sequence
  Description of Artificial Sequence Synthetic polypeptide
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 16 - protein, 491 aa, Artificial Sequence
  Description of Artificial Sequence Synthetic polypeptide
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYAQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 17 - protein, 390 aa, Artificial Sequence
  Description of Artificial Sequence Synthetic polypeptide
MIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 18 - protein, 390 aa, Artificial Sequence
  Description of Artificial Sequence Synthetic polypeptide
MIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYAQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 19 - protein, 1985 aa, Artificial Sequence
  Description of Artificial Sequence Synthetic polypeptide
MAPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKDAKSLTAWSRTLVTFKDVFVDFTREEWKLLDTAQQIVYRNVMLENYKNLVSLGYQLTKPDVILRLEKGEEPWLVSLGSGSPAAKRVKLDEDPAAKRVKLDLIKTSGSGMCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 20 - protein, 1985 aa, Artificial Sequence
  Description of Artificial Sequence Synthetic polypeptide
MAPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKDAKSLTAWSRTLVTFKDVFVDFTREEWKLLDTAQQIVYRNVMLENYKNLVSLGYQLTKPDVILRLEKGEEPWLVSLGSGSPAAKRVKLDEDPAAKRVKLDLIKTSGSGMCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYAQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 21 - protein, 1884 aa, Artificial Sequence
  Description of Artificial Sequence Synthetic polypeptide
MAPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKDAKSLTAWSRTLVTFKDVFVDFTREEWKLLDTAQQIVYRNVMLENYKNLVSLGYQLTKPDVILRLEKGEEPWLVSLGSGSPAAKRVKLDEDPAAKRVKLDLIKTSGSGMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 22 - protein, 1884 aa, Artificial Sequence
  Description of Artificial Sequence Synthetic polypeptide
MAPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKDAKSLTAWSRTLVTFKDVFVDFTREEWKLLDTAQQIVYRNVMLENYKNLVSLGYQLTKPDVILRLEKGEEPWLVSLGSGSPAAKRVKLDEDPAAKRVKLDLIKTSGSGMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYAQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 23 - protein, 1596 aa, Artificial Sequence
  Description of Artificial Sequence Synthetic polypeptide
MAPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKDAKSLTAWSRTLVTFKDVFVDFTREEWKLLDTAQQIVYRNVMLENYKNLVSLGYQLTKPDVILRLEKGEEPWLVSLGSGSPAAKRVKLDEDPAAKRVKLDLIKTSGSGMTAMEESQSDISLKRALPTCTSASPPQKKKPLDGEYFTLKIRGRKRFEMFRELNEALELKDAHATEESGDSRAHSSYLKTKKGQSTSRHKKTMVKKVGPDSD

SEQ ID NO: 24 - protein, 1599 aa, Artificial Sequence
  Description of Artificial Sequence Synthetic polypeptide
MAPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKDAKSLTAWSRTLVTFKDVFVDFTREEWKLLDTAQQIVYRNVMLENYKNLVSLGYQLTKPDVILRLEKGEEPWLVSLGSGSPAAKRVKLDEDPAAKRVKLDLIKTSGSGMEEPQSDPSVEPPLSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 25 - protein, 6 aa, Artificial Sequence
  Description of Artificial Sequence Synthetic 6xHis tag
HHHHHH

SEQ ID NO: 26 - protein, 8 aa, Artificial Sequence
  Description of Artificial Sequence Synthetic 8xHis tag
HHHHHHHH

SEQ ID NO: 27 - DNA, 21 bp, Artificial Sequence
  Description of Artificial Sequence Synthetic oligonucleotide
  modified_base (1)..(14): a, c, t or g
  modified_base (15)..(16): a, c, t, g, unknown or other
nnnnnnnnnnnnnnnnagaaw

SEQ ID NO: 28 - DNA, 13 bp, Artificial Sequence
  Description of Artificial Sequence Synthetic oligonucleotide
  modified_base (1)..(6): a, c, t or g
  modified_base (7)..(8): a, c, t, g, unknown or other
nnnnnnnnagaaw

SEQ ID NO: 29 - DNA, 21 bp, Artificial Sequence
  Description of Artificial Sequence Synthetic oligonucleotide
  modified_base (1)..(14): a, c, t or g
  modified_base (15)..(16): a, c, t, g, unknown or other
nnnnnnnnnnnnnnnnagaaw

SEQ ID NO: 30 - DNA, 18 bp, Artificial Sequence
  Description of Artificial Sequence Synthetic oligonucleotide
  modified_base (1)..(11): a, c, t or g
  modified_base (12)..(13): a, c, t, g, unknown or other
nnnnnnnnnnnnnagaaw

SEQ ID NO: 31 - protein, 9 aa, Unknown
  Description of Unknown ′LAGLIDADG′ family motif peptide
LAGLIDADG

SEQ ID NO: 32 - DNA, 23 bp, Artificial Sequence
  Description of Artificial Sequence Synthetic oligonucleotide
aatgtcatggaggatcacgctgg

SEQ ID NO: 33 - DNA, 23 bp, Artificial Sequence
  Description of Artificial Sequence Synthetic oligonucleotide
aatgtcatggaggatcacgctgg

SEQ ID NO: 34 - DNA, 23 bp, Artificial Sequence
  Description of Artificial Sequence Synthetic oligonucleotide
gatgtcacggaggatcacgctgg

SEQ ID NO: 35 - DNA, 23 bp, Artificial Sequence
  Description of Artificial Sequence Synthetic oligonucleotide
gagctaatggaagatcacgtcag

SEQ ID NO: 36 - DNA, 23 bp, Artificial Sequence
  Description of Artificial Sequence Synthetic oligonucleotide
gtattgttggaatatcacgcaag

SEQ ID NO: 37 - DNA, 23 bp, Artificial Sequence
  Description of Artificial Sequence Synthetic oligonucleotide
gaaatgggggaaaatcacgctag

SEQ ID NO: 38 - DNA, 20 bp, Artificial Sequence
  Description of Artificial Sequence Synthetic primer
gagctgcttttccagatgct

SEQ ID NO: 39 - DNA, 20 bp, Artificial Sequence
  Description of Artificial Sequence Synthetic primer
ccaatgtggggactctttgt

SEQ ID NO: 40 - DNA, 25 bp, Artificial Sequence
  Description of Artificial Sequence Synthetic primer
gttgataaccccttaaattgtttca

SEQ ID NO: 41 - DNA, 22 bp, Artificial Sequence
  Description of Artificial Sequence Synthetic primer
ggagtcttcaattaaggtgcca

SEQ ID NO: 42 - DNA, 23 bp, Artificial Sequence
  Description of Artificial Sequence Synthetic primer
aaatccttttattgtggtgggaa

SEQ ID NO: 43 - DNA, 20 bp, Artificial Sequence
  Description of Artificial Sequence Synthetic primer
ccttgagtgccgtaaccaac

SEQ ID NO: 44 - DNA, 16 bp, Artificial Sequence
  Description of Artificial Sequence Synthetic primer
gcgcccacagccatgt

SEQ ID NO: 45 - DNA, 15 bp, Artificial Sequence
  Description of Artificial Sequence Synthetic primer
gaccgcggcgggaca

SEQ ID NO: 46 - DNA, 24 bp, Artificial Sequence
  Description of Artificial Sequence Synthetic primer
aaactctttttaaccctgttttcc

SEQ ID NO: 47 - DNA, 20 bp, Artificial Sequence
  Description of Artificial Sequence Synthetic primer
caattaaggtgccagggcta

SEQ ID NO: 48 - DNA, 25 bp, Artificial Sequence
  Description of Artificial Sequence Synthetic primer
aattcctgggtggtattatatgttg

SEQ ID NO: 49 - DNA, 26 bp, Artificial Sequence
  Description of Artificial Sequence Synthetic primer
ttctactttggagtcttcaattaagg

SEQ ID NO: 50 - DNA, 23 bp, Artificial Sequence
  Description of Artificial Sequence Synthetic primer
aagggtcactaatttcgatcagt

SEQ ID NO: 51 - DNA, 21 bp, Artificial Sequence
  Description of Artificial Sequence Synthetic primer
tgtgatgacaaaccttggaga

SEQ ID NO: 52 - DNA, 23 bp, Artificial Sequence
  Description of Artificial Sequence Synthetic primer
cagttccctaacaaagagcctct

SEQ ID NO: 53 - DNA, 20 bp, Artificial Sequence
  Description of Artificial Sequence Synthetic primer
aacttcgccatgtgatgaca

SEQ ID NO: 54 - DNA, 21 bp, Artificial Sequence
  Description of Artificial Sequence Synthetic primer
aaagtgaagaatgaggcgtga

SEQ ID NO: 55 - DNA, 19 bp, Artificial Sequence
  Description of Artificial Sequence Synthetic primer
gctaggatggcaatggcta

SEQ ID NO: 56 - DNA, 21 bp, Artificial Sequence
  Description of Artificial Sequence Synthetic primer
gccaggataaaggagatgaag

SEQ ID NO: 57 - DNA, 19 bp, Artificial Sequence
  Description of Artificial Sequence Synthetic primer
gactaccccacagccatga

SEQ ID NO: 58 - DNA, 20 bp, Artificial Sequence
  Description of Artificial Sequence Synthetic primer
aagcaacctaacccctgtct

SEQ ID NO: 59 - DNA, 19 bp, Artificial Sequence
  Description of Artificial Sequence Synthetic primer
accctcttcccctccttca

SEQ ID NO: 60 - DNA, 20 bp, Artificial Sequence
  Description of Artificial Sequence Synthetic primer
gaaaatgggctagatggagt

SEQ ID NO: 61 - DNA, 19 bp, Artificial Sequence
  Description of Artificial Sequence Synthetic primer
aggtcttggcttcagcatc

SEQ ID NO: 62 - DNA, 18 bp, Artificial Sequence
  Description of Artificial Sequence Synthetic primer
tcgccacaccccctattc

SEQ ID NO: 63 - DNA, 18 bp, Artificial Sequence
  Description of Artificial Sequence Synthetic primer
cgggggcagtgaaggatg

SEQ ID NO: 64 - DNA, 19 bp, Artificial Sequence
  Description of Artificial Sequence Synthetic primer
caacccggacttggtgttc

SEQ ID NO: 65 - DNA, 18 bp, Artificial Sequence
  Description of Artificial Sequence Synthetic primer
tggaagcgatgacccaga

SEQ ID NO: 66 - DNA, 20 bp, Artificial Sequence
  Description of Artificial Sequence Synthetic primer
gtagcttacctttgagccat

SEQ ID NO: 67 - DNA, 20 bp, Artificial Sequence
  Description of Artificial Sequence Synthetic primer
tgcagggaacaacctatctt

SEQ ID NO: 68 - DNA, 24 bp, Artificial Sequence
  Description of Artificial Sequence Synthetic primer
aagatatacgacttgaagatgaag

SEQ ID NO: 69 - DNA, 20 bp, Artificial Sequence
  Description of Artificial Sequence Synthetic primer
agtaccatcaccccaaacat

SEQ ID NO: 70 - DNA, 20 bp, Artificial Sequence
  Description of Artificial Sequence Synthetic primer
agcaaaaccagccaaatcat

SEQ ID NO: 71 - DNA, 20 bp, Artificial Sequence
  Description of Artificial Sequence Synthetic primer
tgtcttccaaattgacccaa

SEQ ID NO: 72 - DNA, 22 bp, Artificial Sequence
  Description of Artificial Sequence Synthetic primer
aggaaaaggagtcagagaagca

SEQ ID NO: 73 - DNA, 19 bp, Artificial Sequence
  Description of Artificial Sequence Synthetic primer
atgtcttccaggctggcaa

SEQ ID NO: 74 - DNA, 21 bp, Artificial Sequence
  Description of Artificial Sequence Synthetic primer
attcccatacagggaggatga

SEQ ID NO: 75 - DNA, 21 bp, Artificial Sequence
  Description of Artificial Sequence Synthetic primer
aaagcacaaagcaagcacttg

SEQ ID NO: 76 - DNA, 23 bp, Artificial Sequence
  Description of Artificial Sequence Synthetic primer
tgctttctcagacttctcatcgt

SEQ ID NO: 77 - DNA, 20 bp, Artificial Sequence
  Description of Artificial Sequence Synthetic primer
gcaacagacacagtccctga

SEQ ID NO: 78 - DNA, 21 bp, Artificial Sequence
  Description of Artificial Sequence Synthetic primer
actcctcagaacttatcctct

SEQ ID NO: 79 - DNA, 20 bp, Artificial Sequence
  Description of Artificial Sequence Synthetic primer
ccctcttggtcttggtgcat

SEQ ID NO: 80 - DNA, 20 bp, Artificial Sequence
  Description of Artificial Sequence Synthetic primer
agctggtccttctgtcatca

SEQ ID NO: 81 - DNA, 21 bp, Artificial Sequence
  Description of Artificial Sequence Synthetic primer
gagtggaaaccagagatcagg

SEQ ID NO: 82 - DNA, 22 bp, Artificial Sequence
  Description of Artificial Sequence Synthetic primer
caaagcggaaacaaacagaaag

SEQ ID NO: 83 - DNA, 20 bp, Artificial Sequence
  Description of Artificial Sequence Synthetic primer
ctcaccatctataacgccaa

SEQ ID NO: 84 - DNA, 18 bp, Artificial Sequence
  Description of Artificial Sequence Synthetic primer
cacctccgttgaggcaca

SEQ ID NO: 85 - DNA, 18 bp, Artificial Sequence
  Description of Artificial Sequence Synthetic primer
tcaagctcaccctggcct

SEQ ID NO: 86 - DNA, 24 bp, Artificial Sequence
  Description of Artificial Sequence Synthetic primer
gcgttagcataggaaaagacaatg

SEQ ID NO: 87 - DNA, 23 bp, Artificial Sequence
  Description of Artificial Sequence Synthetic primer
tttggcatattctacaggcaaag

SEQ ID NO: 88 - DNA, 20 bp, Artificial Sequence
  Description of Artificial Sequence Synthetic primer
caggctccactagtgcttca

SEQ ID NO: 89 - DNA, 20 bp, Artificial Sequence
  Description of Artificial Sequence Synthetic primer
ccattcaaagcagcagtgat

SEQ ID NO: 90 - DNA, 20 bp, Artificial Sequence
  Description of Artificial Sequence Synthetic primer
caggagcacctttcttgtgc

SEQ ID NO: 91 - DNA, 20 bp, Artificial Sequence
  Description of Artificial Sequence Synthetic primer
gtgtctgtgccacaggtgaa

SEQ ID NO: 92 - DNA, 18 bp, Artificial Sequence
  Description of Artificial Sequence Synthetic primer
caggcgggcctggtggat

SEQ ID NO: 93 - DNA, 18 bp, Artificial Sequence
  Description of Artificial Sequence Synthetic primer
cccctgccaccccgttac

SEQ ID NO: 94 - DNA, 20 bp, Artificial Sequence
  Description of Artificial Sequence Synthetic primer
tttccttagccctggaaact

SEQ ID NO: 95 - DNA, 20 bp, Artificial Sequence
  Description of Artificial Sequence Synthetic primer
gcatccactcatggtgatct

SEQ ID NO: 96 - DNA, 19 bp, Artificial Sequence
  Description of Artificial Sequence Synthetic primer
ccctgtgccttgtcactca

SEQ ID NO: 97 - DNA, 20 bp, Artificial Sequence
  Description of Artificial Sequence Synthetic primer
tgtatgctgcatgaggctca

SEQ ID NO: 98 - DNA, 20 bp, Artificial Sequence
  Description of Artificial Sequence Synthetic primer
caagatctctggatcctgca

SEQ ID NO: 99 - DNA, 23 bp, Artificial Sequence
  Description of Artificial Sequence Synthetic primer
aggaaaggatgtatcagcataag

SEQ ID NO: 100 - DNA, 25 bp, Artificial Sequence
  Description of Artificial Sequence Synthetic primer
aactccttttcaactttgctaccat

SEQ ID NO: 101 - DNA, 20 bp, Artificial Sequence
  Description of Artificial Sequence Synthetic primer
cgaccatctgcgtatgcaca

SEQ ID NO: 102 - DNA, 23 bp, Artificial Sequence
  Description of Artificial Sequence Synthetic primer
cagatatggaagtgaagcaaatc

SEQ ID NO: 103 - DNA, 21 bp, Artificial Sequence
  Description of Artificial Sequence Synthetic primer
acctttcttctttcctctgca

SEQ ID NO: 104 - DNA, 20 bp, Artificial Sequence
  Description of Artificial Sequence Synthetic primer
gctgatcttgaagccaccat

SEQ ID NO: 105 - DNA, 23 bp, Artificial Sequence
  Description of Artificial Sequence Synthetic primer
agagagaaggaaaacctatggaa

SEQ ID NO: 106 - DNA, 21 bp, Artificial Sequence
  Description of Artificial Sequence Synthetic primer
tcagcttttcatttcagccta

SEQ ID NO: 107 - DNA, 23 bp, Artificial Sequence
  Description of Artificial Sequence Synthetic primer
gtcaacctttccaaatgatctag

SEQ ID NO: 108 - DNA, 22 bp, Artificial Sequence
  Description of Artificial Sequence Synthetic primer
tacacatctacctatgcacagc

SEQ ID NO: 109 - DNA, 20 bp, Artificial Sequence
  Description of Artificial Sequence Synthetic primer
ctcataatgcagttcccact

SEQ ID NO: 110 - DNA, 19 bp, Artificial Sequence
  Description of Artificial Sequence Synthetic primer
gcctgagccatcttgttga

SEQ ID NO: 111 - DNA, 21 bp, Artificial Sequence
  Description of Artificial Sequence Synthetic primer
aactgaaagcacggtgttact

SEQ ID NO: 112 - DNA, 20 bp, Artificial Sequence
  Description of Artificial Sequence Synthetic primer
ggacacgcacgtcatatttg

SEQ ID NO: 113 - DNA, 19 bp, Artificial Sequence
  Description of Artificial Sequence Synthetic primer
cacagctgccctatcctga

SEQ ID NO: 114 - DNA, 18 bp, Artificial Sequence
  Description of Artificial Sequence Synthetic primer
gtagcctgcatcgccgta

SEQ ID NO: 115 - DNA, 19 bp, Artificial Sequence
  Description of Artificial Sequence Synthetic primer
taccacagccgagccttgt

SEQ ID NO: 116 - DNA, 20 bp, Artificial Sequence
  Description of Artificial Sequence Synthetic primer
cttgcatgtgttccctgtct

SEQ ID NO: 117 - DNA, 22 bp, Artificial Sequence
  Description of Artificial Sequence Synthetic primer
agtccactcacagtgctgtctt

SEQ ID NO: 118 - DNA, 21 bp, Artificial Sequence
  Description of Artificial Sequence Synthetic primer
atgggccctttgttattctct

SEQ ID NO: 119 - DNA, 22 bp, Artificial Sequence
  Description of Artificial Sequence Synthetic primer
aagtgtactttgatgcacccac

SEQ ID NO: 120 - DNA, 20 bp, Artificial Sequence
  Description of Artificial Sequence Synthetic primer
atgccatatgggagcaatgt

SEQ ID NO: 121 - DNA, 20 bp, Artificial Sequence
  Description of Artificial Sequence Synthetic primer
gagagaaccgtgccacagac

SEQ ID NO: 122 - DNA, 20 bp, Artificial Sequence
  Description of Artificial Sequence Synthetic primer
agtttgttggtcccaggttg

SEQ ID NO: 123 - DNA, 20 bp, Artificial Sequence
  Description of Artificial Sequence Synthetic primer
tcagattgtccctggaggtc

SEQ ID NO: 124 - DNA, 20 bp, Artificial Sequence
  Description of Artificial Sequence Synthetic primer
gcagtcactgcaatgaattg

SEQ ID NO: 125 - DNA, 26 bp, Artificial Sequence
  Description of Artificial Sequence Synthetic primer
ccactaagaggctttttagatttagc

SEQ ID NO: 126 - DNA, 20 bp, Artificial Sequence
  Description of Artificial Sequence Synthetic primer
tcagaacccagcttctctca

SEQ ID NO: 127 - DNA, 22 bp, Artificial Sequence
  Description of Artificial Sequence Synthetic primer
gccacagtgaatgtcacaattc

SEQ ID NO: 128 - DNA, 20 bp, Artificial Sequence
  Description of Artificial Sequence Synthetic primer
cacacacctgctggtagctt

SEQ ID NO: 129 - DNA, 19 bp, Artificial Sequence
  Description of Artificial Sequence Synthetic primer
ctccccttcctcctcagaa

SEQ ID NO: 130 - DNA, 20 bp, Artificial Sequence
  Description of Artificial Sequence Synthetic primer
gaggcttggacaattccaga

SEQ ID NO: 131 - DNA, 20 bp, Artificial Sequence
  Description of Artificial Sequence Synthetic primer
actccctcttctgtgacctc

SEQ ID NO: 132 - DNA, 20 bp, Artificial Sequence
  Description of Artificial Sequence Synthetic primer
gcaactgaccgtgcaagtca

SEQ ID NO: 133 - DNA, 19 bp, Artificial Sequence
  Description of Artificial Sequence Synthetic primer
cagaatgccagaggctgct

SEQ ID NO: 134 - DNA, 20 bp, Artificial Sequence
  Description of Artificial Sequence Synthetic primer
aaccagccctgtcgtctctc

SEQ ID NO: 135 - DNA, 19 bp, Artificial Sequence
  Description of Artificial Sequence Synthetic primer
tttgccaactggccaagac

SEQ ID NO: 136 - DNA, 23 bp, Artificial Sequence
  Description of Artificial Sequence Synthetic primer
agcaatcagtgaggaatcagagg

SEQ ID NO: 137 - DNA, 20 bp, Artificial Sequence
  Description of Artificial Sequence Synthetic primer
gctgtgggttgattccacac

SEQ ID NO: 138 - DNA, 20 bp, Artificial Sequence
  Description of Artificial Sequence Synthetic primer
aaggagattctatcagcaga

SEQ ID NO: 139 - DNA, 20 bp, Artificial Sequence
  Description of Artificial Sequence Synthetic primer
tgagcttccagccagggatt

SEQ ID NO: 140 - DNA, 20 bp, Artificial Sequence
  Description of Artificial Sequence Synthetic primer
tgacattgcagaaaggagat

SEQ ID NO: 141 - DNA, 20 bp, Artificial Sequence
  Description of Artificial Sequence Synthetic primer
caacaagctcgctgtgttct

SEQ ID NO: 142 - DNA, 23 bp, Artificial Sequence
  Description of Artificial Sequence Synthetic primer
ttctccactttcagactggtgat

SEQ ID NO: 143 - DNA, 23 bp, Artificial Sequence
  Description of Artificial Sequence Synthetic primer
agttcaaatatgacgacgcagaa

SEQ ID NO: 144 - DNA, 21 bp, Artificial Sequence
  Description of Artificial Sequence Synthetic primer
gtatttgaactactggccttg

SEQ ID NO: 145 - DNA, 21 bp, Artificial Sequence
  Description of Artificial Sequence Synthetic primer
ccactttcttgtcagttccaa

SEQ ID NO: 146 - DNA, 20 bp, Artificial Sequence
  Description of Artificial Sequence Synthetic primer
cctcggtgctagaatcttga

SEQ ID NO: 147 - DNA, 20 bp, Artificial Sequence
  Description of Artificial Sequence Synthetic primer
tactttccttcattgtgcct

SEQ ID NO: 148 - DNA, 20 bp, Artificial Sequence
  Description of Artificial Sequence Synthetic primer
ggaggctcgttcacttacaa

SEQ ID NO: 149 - DNA, 23 bp, Artificial Sequence
  Description of Artificial Sequence Synthetic primer
gagtcactattaatttggattca

SEQ ID NO: 150 - DNA, 20 bp, Artificial Sequence
  Description of Artificial Sequence Synthetic primer
cctggccattgtcactttgc

SEQ ID NO: 151 - DNA, 22 bp, Artificial Sequence
  Description of Artificial Sequence Synthetic primer
ccacgtaacctgagaagggaat

SEQ ID NO: 152 - DNA, 20 bp, Artificial Sequence
  Description of Artificial Sequence Synthetic primer
aactctgccctctaacgctg

SEQ ID NO: 153 - DNA, 27 bp, Artificial Sequence
  Description of Artificial Sequence Synthetic primer
ctatcgattcacacaaaaaaccaacac

SEQ ID NO: 154 - DNA, 20 bp, Artificial Sequence
  Description of Artificial Sequence Synthetic primer
ggaatgggccgataggttcc

SEQ ID NO: 155 - DNA, 20 bp, Artificial Sequence
  Description of Artificial Sequence Synthetic primer
gaatccctcctctctgaacc

SEQ ID NO: 156 - DNA, 20 bp, Artificial Sequence
  Description of Artificial Sequence Synthetic oligonucleotide
gtacatcttacgaatcactg

SEQ ID NO: 157 - DNA, 20 bp, Artificial Sequence
  Description of Artificial Sequence Synthetic oligonucleotide
attatcgacctaaagcaact

SEQ ID NO: 158 - DNA, 20 bp, Artificial Sequence
  Description of Artificial Sequence Synthetic oligonucleotide
tgagcacaatgaaggcaagt

SEQ ID NO: 159 - DNA, 20 bp, Artificial Sequence
  Description of Artificial Sequence Synthetic oligonucleotide
atagtcatgaggtagctgcg

SEQ ID NO: 160 - DNA, 20 bp, Artificial Sequence
  Description of Artificial Sequence Synthetic oligonucleotide
ggatcgtcgcaattctccca

SEQ ID NO: 161 - DNA, 20 bp, Artificial Sequence
  Description of Artificial Sequence Synthetic oligonucleotide
ttatgacagaaagtttctgt

SEQ ID NO: 162 - DNA, 20 bp, Artificial Sequence
  Description of Artificial Sequence Synthetic oligonucleotide
gatggtcaagtaaactatga

SEQ ID NO: 163 - DNA, 20 bp, Artificial Sequence
  Description of Artificial Sequence Synthetic oligonucleotide
gaagcagatattgatggtga

SEQ ID NO: 164 - DNA, 20 bp, Artificial Sequence
  Description of Artificial Sequence Synthetic oligonucleotide
agaagttgatgaaatgatca

SEQ ID NO: 165 - DNA, 20 bp, Artificial Sequence
  Description of Artificial Sequence Synthetic oligonucleotide
aggcgaattgtgatccaccg

SEQ ID NO: 166 - DNA, 20 bp, Artificial Sequence
  Description of Artificial Sequence Synthetic oligonucleotide
atgggtcgtcaccgatgtgt

SEQ ID NO: 167 - DNA, 20 bp, Artificial Sequence
  Description of Artificial Sequence Synthetic oligonucleotide
gtaacaaagatcatcgaagg

SEQ ID NO: 168 - DNA, 20 bp, Artificial Sequence
  Description of Artificial Sequence Synthetic oligonucleotide
gatgacccagatatcggtag

SEQ ID NO: 169 - DNA, 20 bp, Artificial Sequence
  Description of Artificial Sequence Synthetic oligonucleotide
gctgctgcgaggcgaatagg

SEQ ID NO: 170 - DNA, 20 bp, Artificial Sequence
  Description of Artificial Sequence Synthetic oligonucleotide
agtaactgcatgcttgctgg

SEQ ID NO: 171 - DNA, 20 bp, Artificial Sequence
  Description of Artificial Sequence Synthetic oligonucleotide
ggagaatcccaatccataaa

SEQ ID NO: 172 - DNA, 20 bp, Artificial Sequence
  Description of Artificial Sequence Synthetic oligonucleotide
acagcacaagaaatgtgtat

SEQ ID NO: 173 - DNA, 20 bp, Artificial Sequence
  Description of Artificial Sequence Synthetic oligonucleotide
cctgataaaaaggacatgca

SEQ ID NO: 174 - DNA, 20 bp, Artificial Sequence
  Description of Artificial Sequence Synthetic oligonucleotide
accaaagagaatgggatcaa

SEQ ID NO: 175 - DNA, 20 bp, Artificial Sequence
  Description of Artificial Sequence Synthetic oligonucleotide
gaggctgtagcagtcctgaa

SEQ ID NO: 176 - DNA, 20 bp, Artificial Sequence
  Description of Artificial Sequence Synthetic oligonucleotide
gggaatctgacgatttcctg

SEQ ID NO: 177 - DNA, 20 bp, Artificial Sequence
  Description of Artificial Sequence Synthetic oligonucleotide
catggagaggtcaatccgca

SEQ ID NO: 178 - DNA, 20 bp, Artificial Sequence
  Description of Artificial Sequence Synthetic oligonucleotide
ctccagcacgttgaacaacg

SEQ ID NO: 179 - DNA, 20 bp, Artificial Sequence
  Description of Artificial Sequence Synthetic oligonucleotide
tgctgagtatgaggtctacg

SEQ ID NO: 180 - DNA, 20 bp, Artificial Sequence
  Description of Artificial Sequence Synthetic oligonucleotide
aaagatcttcacgttgacgg

SEQ ID NO: 181 - DNA, 20 bp, Artificial Sequence
  Description of Artificial Sequence Synthetic oligonucleotide
tgagtatgaggtctacgtgg

SEQ ID NO: 182 - DNA, 20 bp, Artificial Sequence
  Description of Artificial Sequence Synthetic oligonucleotide
gtccacgggcaggacctgcg

SEQ ID NO: 183 - DNA, 20 bp, Artificial Sequence
  Description of Artificial Sequence Synthetic oligonucleotide
acagtgcgtgtagttcggcg

SEQ ID NO: 184 - DNA, 20 bp, Artificial Sequence
  Description of Artificial Sequence Synthetic oligonucleotide
gatgcttcacactgggaaat

SEQ ID NO: 185 - DNA, 20 bp, Artificial Sequence
  Description of Artificial Sequence Synthetic oligonucleotide
gtgaacatgatccgccaagg

SEQ ID NO: 186 - DNA, 20 bp, Artificial Sequence
  Description of Artificial Sequence Synthetic oligonucleotide
cctgacatcctatgacagcg

SEQ ID NO: 187 - DNA, 20 bp, Artificial Sequence
  Description of Artificial Sequence Synthetic oligonucleotide
tgtcggcacctcgatacagg

SEQ ID NO: 188 - DNA, 20 bp, Artificial Sequence
  Description of Artificial Sequence Synthetic oligonucleotide
gggcaataatcaggacggag

SEQ ID NO: 189 - DNA, 20 bp, Artificial Sequence
  Description of Artificial Sequence Synthetic oligonucleotide
caaccagttgcagcatacga

SEQ ID NO: 190 - DNA, 20 bp, Artificial Sequence
  Description of Artificial Sequence Synthetic oligonucleotide
gttatgttggatgaacaagg

SEQ ID NO: 191 - DNA, 20 bp, Artificial Sequence
  Description of Artificial Sequence Synthetic oligonucleotide
tggagaagctaaagttcgtg

SEQ ID NO: 192 - DNA, 20 bp, Artificial Sequence
  Description of Artificial Sequence Synthetic oligonucleotide
gaagaaagacctgtccctgg

SEQ ID NO: 193 - DNA, 20 bp, Artificial Sequence
  Description of Artificial Sequence Synthetic oligonucleotide
tgttgatgttgcgaggttcc

SEQ ID NO: 194 - DNA, 20 bp, Artificial Sequence
  Description of Artificial Sequence Synthetic oligonucleotide
cagcagtacaacgtaccccg

SEQ ID NO: 195 - DNA, 20 bp, Artificial Sequence
  Description of Artificial Sequence Synthetic oligonucleotide
gtcaccaatgagcggcatgg

SEQ ID NO: 196 - DNA, 20 bp, Artificial Sequence
  Description of Artificial Sequence Synthetic oligonucleotide
ccaggacatcgcaagtgtcg

SEQ ID NO: 197 - DNA, 20 bp, Artificial Sequence
  Description of Artificial Sequence Synthetic oligonucleotide
tctccttaaacacgaaccac

SEQ ID NO: 198 - DNA, 20 bp, Artificial Sequence
  Description of Artificial Sequence Synthetic oligonucleotide
gctagttagctcatcggcat

SEQ ID NO: 199 - DNA, 20 bp, Artificial Sequence
  Description of Artificial Sequence Synthetic oligonucleotide
ggagtagaggaaggcaaaca

SEQ ID NO: 200 - DNA, 20 bp, Artificial Sequence
  Description of Artificial Sequence Synthetic oligonucleotide
ggcgtgacaagccaccgctg

SEQ ID NO: 201 - DNA, 20 bp, Artificial Sequence
  Description of Artificial Sequence Synthetic oligonucleotide
agaaccgtgccacagaccaa

SEQ ID NO: 202 - DNA, 20 bp, Artificial Sequence
  Description of Artificial Sequence Synthetic oligonucleotide
agttacagaggatggcgtgg

SEQ ID NO: 203 - DNA, 20 bp, Artificial Sequence
  Description of Artificial Sequence Synthetic oligonucleotide
caactacaagacccgcgccg

SEQ ID NO: 204 - DNA, 20 bp, Artificial Sequence
  Description of Artificial Sequence Synthetic oligonucleotide
cggccatgatatagacgttg

SEQ ID NO: 205 - DNA, 20 bp, Artificial Sequence
  Description of Artificial Sequence Synthetic oligonucleotide
cgatgcccttcagctcgatg

SEQ ID NO: 206 - DNA, 20 bp, Artificial Sequence
  Description of Artificial Sequence Synthetic oligonucleotide
gaagtgatggaagatcacgc

SEQ ID NO: 207 - DNA, 20 bp, Artificial Sequence
  Description of Artificial Sequence Synthetic oligonucleotide
gaagggacagaagatgacag

SEQ ID NO: 208 - DNA, 20 bp, Artificial Sequence
  Description of Artificial Sequence Synthetic oligonucleotide
gaagggacagaagatgacag

SEQ ID NO: 209 - DNA, 20 bp, Artificial Sequence
  Description of Artificial Sequence Synthetic oligonucleotide
gagcgctgctcagatagcga